(19) United States
(12) Patent Application Publication
(10) Pub. No.: US 2013/0109693 A1
(43) Pub. Date: May 2, 2013

(54) DERIVATIVES OF PYRIDO [3,2-D] PYRIMIDINE, METHODS FOR PREPARATION THEREOF AND THERAPEUTIC USES THEREOF

(71) Applicants: Sylvain Routier, Tigy (FR); Gérald Guillaumet, Saint Jean Le Blanc (FR); Abdellatif Tikad, Namur (BE); Oussama Dehbi, Orleans (FR)

(72) Inventors: Sylvain Routier, Tigy (FR); Gérald Guillaumet, Saint Jean Le Blanc (FR); Abdellatif Tikad, Namur (BE); Oussama Dehbi, Orleans (FR)

(73) Assignees: UNIVERSITE D'ORLEANS, Orleans Cedex 2 (FR); CENTRE NATIONAL DE LA RECHERCHE SCIENTIFIQUE (C.N.R.S), Paris (FR)

(21) Appl. No.: 13/695,001

(22) PCT Filed: Apr. 28, 2011
(86) PCT No.: PCT/FR2011/050959

§ 371 Date: Dec. 20, 2012

(30) Foreign Application Priority Data
Apr. 28, 2010 (FR) 1053277

(51) Int. Cl. C07D 471/04

(52) U.S. Cl. CPC C07D 471/04
USPC 514/245; 514/264.1; 544/279; 514/252.16; 544/212

(57) ABSTRACT

The present invention relates to a compound of the following general formula (I):

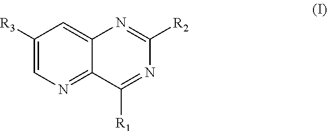

wherein:
   R₁ is notably H, a halogen or an aryl group,
   R₂ is notably a halogen or an aryl group,
   R₃ is notably a halogen, or an aryl or heteroaryl group, as well as to its pharmaceutically acceptable salts, its hydrates or its polymorphic crystalline structures, its racemates, diastereoisomers or enantiomers,
      except the compound 1-(2,4-diaminopyrido[3,2-d]pyrimidin-7-yl)-3,6,6-trimethyl-6,7-dihydro-1H-indol-4(5H)-one.

The object of the present invention is novel derivatives of the pyrido[3,2-d]pyrimidine type and their preparation methods. The object is also the therapeutic uses of said novel derivatives notably as inhibitors of kinases.

Protein kinases catalyze the phosphorylation of residues of the serine, threonine and tyrosine type by using ATP or GTP as a phosphate donor. Kinases are presently part of the most studied biological targets since they are involved in many biological processes. Selective enzymatic inhibition is a preferential strategy for developing new chemotherapies. Protein kinases are among the most popular biological targets in the pharmaceutical industry. The very large number of kinases has made it difficult to determine the specific role of each of them. They are involved in various processes such as growth and cell differentiation, as well as tumoral promotion, orchestration of the cell cycle or the functioning of neuronal cells. Sub- or over-expression of these enzymes has been reported in a wide range of neoplastic and pre-neoplastic tissues. During over-expression, powerful inhibitors of kinases may be useful as anti-proliferative agents.

Considering the importance of these reactions in physiological and cell processes, it is therefore not surprising that malfunctions of these regulation systems become the cause or the consequence of human illnesses. In this respect, a large number of pathologies result from the mutation of kinases and phosphatases. Thus, it is currently recognized that abnormal phosphorylations are responsible for the major part of pathologies such as cancers, diabetes, rheumatoid arthritis, Alzheimer's disease. Presently, erlotinib (Tarceva®) and imatinib (Gleevec®) have been launched on the market as inhibitors of kinases.

Because of the key role played by cyclin-dependant kinases (CDKs) for entering and progressing in the cell cycle, development of pharmacological inhibitors of these enzymes is therefore a major potential therapeutic route for controlling cancer. Very many dysfunctions of CDKs and of their regulators have been described in human tumors. Inhibitors of the enzymatic activity of CDKs may act independently or together with other treatments for limiting tumoral proliferation:

The CDKs 1, 2, 4, 5 and 6 are most frequently over-activated or abnormally regulated in tumors. The inhibitors of CDKs then prove to be powerful anti-proliferative agents stopping the cells in G1 or G2/M.

The inhibitors of CDKs may also be involved in the apoptotic process. Cyclins A, B, D and E and CDKs1 and 2 may play a pro-apoptotic role. The inhibitors of CDKs may then be used in anti-cancer chemotherapy for potentializing the action of cytotoxic drugs, while ensuring protection of healthy cells.

The CDK5 is directly involved in many neurodegenerative processes such as Alzheimer's disease, Parkinson's disease, brain traumas or cerebrovascular strokes. The inhibitors of CDK5 then act as neuroprotectors.

Finally, CDKs seem to be involved in renal polycystoses, and inflammatory processes. The inhibitors of CDKs have very positive effect on animal models of these pathologies.

Glycogen synthase kinase 3 (GSK-3) is a serine/threonine kinase originally identified for its role in the regulation of the metabolism of glycogen. In addition to it being involved in the indirect transduction of insulin and IGF-1 signals, it is very present in the brain and a large body of evidence has been built up for linking GSK-3 to induced neurotoxicity. This suggests that deregulation of GSK-3 may play a key role in the pathogenesis of Alzheimer's disease and therefore GSK-3Beta appeared as a promising therapeutic target for Alzheimer's disease and other neurodegenerations. Chemically, heterocyclic thiadiazolidinones (TDZD) were the only molecules proposed as new drugs for efficient treatment of neurodegenerative disorders where phosphorylation of the tau protein plays a key role like in the case of Alzheimer's disease.

The enzyme DYRK1A is a member of a particular family of kinases (dual-specificity tyrosine phosphorylation-regulated kinase). It catalyzes its self-phosphorylation on serine/threonine and tyrosine residues. It plays an important role in the signaling routes regulating proliferation and is involved in the development of the brain. It appears as a target of choice for treating Alzheimer's disease but also trisomia 21.

The aim of the present invention is to provide novel inhibitors of CDKs, GSK-3 and DYRK1A.

The aim of the present invention is to provide novel inhibitors of CDKs directly and selectively targeting said kinases.

More particularly, the aim of the present invention is to provide specific inhibitors of CDK1, CDK5, GSK3 and DYRK1A kinases.

The present invention relates to compounds of the following general formula (I):

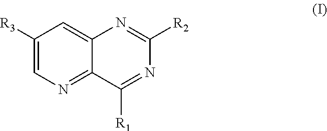

wherein:
R₁ is selected from the group consisting of:
   hydrogen,
   halogens,
   (hetero)aryls comprising from 5 to 30 carbon atoms, optionally substituted,
   groups —NRₐR_{b}, Rₐ and R_{b} being independently selected from the group consisting of hydrogen, alkyls comprising from 1 to 10 carbon atoms, aryls comprising from 5 to 30 carbon atoms and arylalkyls comprising from 6 to 30 carbon atoms, said alkyls, aryls and arylalkyls being optionally substituted,
R₂ is selected from the group consisting of:
   halogens,
   (hetero)aryls comprising from 5 to 30 carbon atoms, optionally substituted,
   groups —NR′ₐR′_{b}, R′ₐ and R′_{b} being independently selected from the group consisting of hydrogen, alkyls comprising from 1 to 10 carbon atoms, aryls comprising from 5 to 30 carbon atoms and arylalkyls comprising from 6 to 30 carbon atoms, said alkyls, aryls and arylalkyls being optionally substituted,
R₃ is selected from the group consisting of:
   halogens,
   (hetero)aryls comprising from 5 to 30 carbon atoms, optionally substituted,
   groups —NR″ₐR″_{b}, R″ₐ and R″_{b} being independently selected from the group consisting of hydrogen, alkyls comprising from 1 to 10 carbon atoms, aryls or heteroaryls comprising from 5 to 30 carbon atoms and arylalkyls comprising from 6 to 30 carbon atoms, said alkyls, aryls and arylalkyls being optionally substituted, and
   groups —N(R″ₐ)COR″_{b}, R″ₐ and R″_{b} being independently selected from the group consisting of hydrogen, alkyls comprising from 1 to 10 carbon atoms, aryls or heteroaryls comprising from 5 to 30 carbon atoms and arylalkyls comprising from 6 to 30 carbon atoms, said alkyls, aryls and arylalkyls being optionally substituted, or R″ₐ and R″_{b} forming with the nitrogen atom bearing R″ₐ and the group CO, a heterocycle comprising from 5 to 10 atoms, and preferably 6 atoms (notably including 1 or 2 heteroatoms, and more particularly 1 or 2 nitrogen atoms), and
   groups —N(R″ₐ)CON(R″_{b}), R″ₐ and R″_{b} being as defined above,
      as well as its pharmaceutically acceptable salts, its hydrates or its polymorphic crystalline structures, its racemates, diastereoisomers or enantiomers.

According to an embodiment, the compounds of the invention are different from the compound, 1-(2,4-diaminopyrido[3,2-d]pyrimidin-7-yl)-3,6,6-trimethyl-6,7-dihydro-1H-indol-4(5H)-one, mentioned in WO 2008/024977:

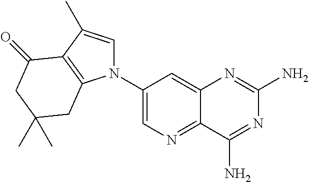 [SMILES: Cc1cn(-c2cnc3c(N)nc(N)nc3c2)c2c1C(=O)CC(C)(C)C2]

According to the present invention the “alkyl” radicals represent saturated hydrocarbon radicals with a straight or branched chain, comprising from 1 to 10 carbon atoms, preferably from 1 to 5 carbon atoms (they may typically be represented by the formula CₙH₂ₙ₊₁, n representing the number of carbon atoms). Mention may notably be made, when they are linear, of the methyl, ethyl, propyl, butyl, pentyl, hexyl, octyl, nonyl and decyl radicals. Mention may notably be made, when they are branched or substituted with one or several alkyl radicals, of the isopropyl, tert-butyl, 2-ethylhexyl, 2-methylbutyl, 2-methylpentyl, 1-methylpentyl and 3-methylheptyl radicals.

The “cycloalkyl” radical is a non-aromatic saturated or partly unsaturated mono-, bi- or tri-cyclic hydrocarbon radical comprising from 3 to 20 carbon atoms, and preferably from 3 to 10 carbon atoms, such as notably cyclopropyl, cyclopentyl, cyclohexyl or adamantyl, as well as the corresponding rings containing one or more unsaturations.

Thus, within the scope of the present invention, the term of “cycloalkyl” also encompasses “heterocycloalkyl” radicals designating non-aromatic, saturated or partly unsaturated, mono- or bicyclic systems with 3 to 8 carbon atoms, comprising one or several heteroatoms selected from N, O or S.

The term of “aryl” designates a mono or bicyclic aromatic hydrocarbon system comprising from 6 to 30, preferably from 6 to 10 carbon atoms. Among aryl radicals, mention may notably be made of the phenyl or naphthyl radical, more particularly substituted with at least one halogen.

When the aryl radical comprises at least one heteroatom, this is referred to as a “heteroaryl” radical. Thus, the term of “heteroaryl” designates an mono- or bicyclic aromatic system comprising one or several heteroatoms selected from nitrogen, oxygen or sulfur, comprising from 5 to 30, preferably from 5 to 10 carbon atoms.

Among heteroaryl radicals, mention may be made of the pyrazinyl, thienyl, oxazolyl, furazanyl, pyrrolyl, 1,2,4-thiadiazolyl, naphthyridinyl, pyridazinyl, quinoxalinyl, phtalazinyl, imidazo[1,2-a]pyridine, imidazo[2,1-b]thiazolyl, cinnolinyl, triazinyl, benzofurazanyl, azaindolyl, benzimidazolyl, benzothienyl, thienopyridyl, thienopyrimidinyl, pyrrolopyridyl, imidazopyridyl, benzoazaindo, 1,2,4-triazinyl, benzothiazolyl, furanyl, imidazolyl, l'indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl, carbazolyl, thiophenyl, benzothiophenyl radicals as well as the corresponding groups originating from their fusion or fusion with the phenyl ring.

The aforementioned “alkyl”, “aryl”, “heteroaryl” and “cycloalkyl” radicals may be substituted with one or several substituents. Among these substituents, mention may be made of the following groups: CHO, amino, amine, hydroxy, thio, halogeno, carboxyl, alkyl (either substituted or not), alkylaryl, alkoxy, alkylthio, alkylcarbonyl, aminocarbonyl, alkylcarboxyl, alkylamino, aryloxy, arylalkoxy, cyano, trifluoromethyl, alkylsulfonyl carboxy or carboxyalkyl.

According to the present invention, the groups Rₐ and R_{b} are selected so that the group —NRₐR_{b} is not a heterocyclic group comprising the nitrogen atom to which the groups Rₐ and R_{b} are bound.

Among the aryl or heteroaryl groups, either substituted or not, mention may more particularly be made of the following groups:

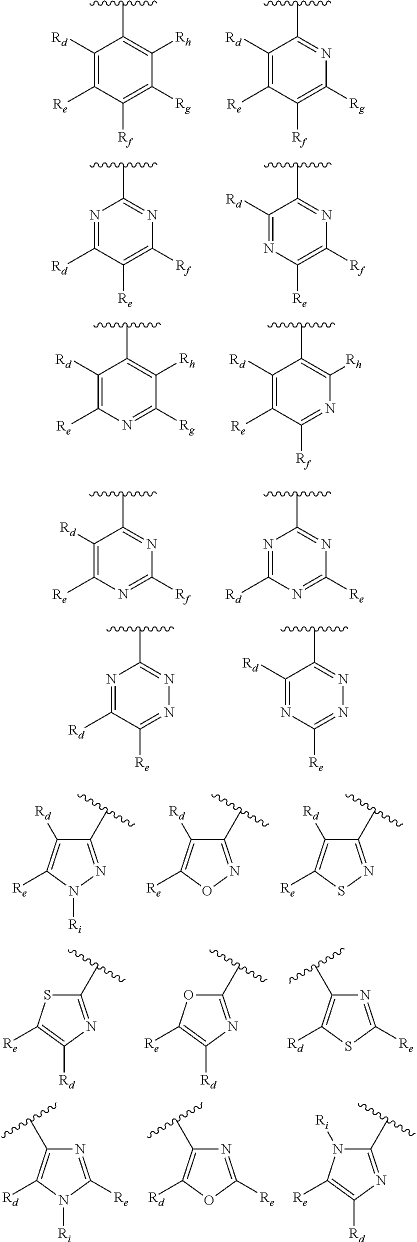

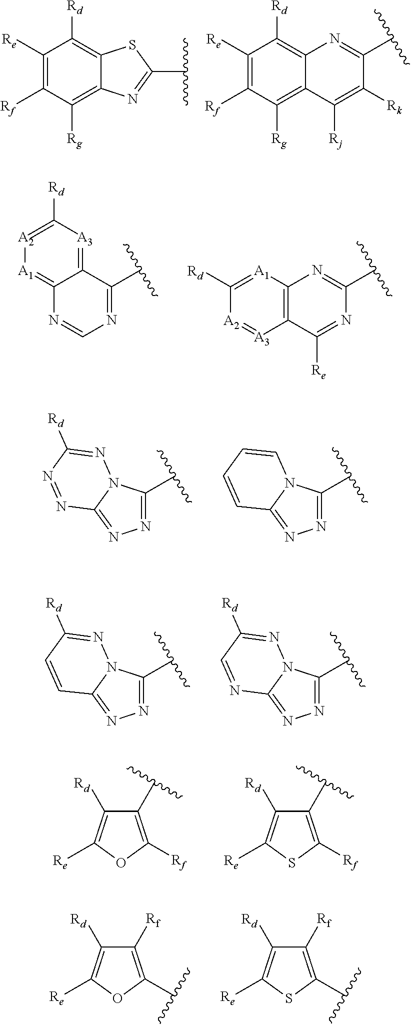

the groups R_{d}, Rₑ, R_{f}, R_{g}, Rₕ, Rⱼ and Rₖ being selected, independently of each other, from the group consisting of the following substituents:
a hydrogen,
a halogen, notably Br, Cl or F,
an alkyl group comprising from 1 to 10 carbon atoms and preferably being a methyl group,
said alkyl group being optionally substituted notably with one or several substituents selected from the group consisting of the following substituents:
   halogens,
   alkenyls or alkynyls comprising from 2 to 10 carbon atoms,
   (hetero)aryls comprising from 5 to 30 carbon atoms,
   COR′_{α}, COOR′_{α}, SR′_{α}, OR′_{α} or NR′_{α}R_{β}, R′_{α} and R_{β} representing independently of each other a hydrogen, an alkyl group comprising from 1 to 10 carbon atoms or a (hetero)aryl comprising from 5 to 30 carbon atoms,
a —CHO group,
a —CN group,
a —NO₂ group,
a —CF₃ group,
a phenyl group,
a —SO₂R′_{α} group R′_{α} being as defined above, notably a SO₂CH₃ group,
a —O—(CH₂)ₙ—O—R′_{α} group, R′_{α} being as defined above and preferably representing an alkyl group, and n representing an integer comprised from 1 to 10, preferably equal to 1, notably a —OCH₂OCH₃ group,
a —CO₂R′_{α} group, R′_{α} being as defined above, notably a group —CO₂H,
a —COR′_{α} group, R′_{α} being as defined above, notably a group —COCH₃,
a —SR′_{α} or —OR′_{α} group, R′_{α} being as defined above, notably a —OH, —OCH₃, —SH, —SCH₃, —O—CH(CH₃)₂, —O—CH₂—CH₂—CH₃ group,
a —NR′_{α}R_{β} group, R′_{α} and R_{β} being as defined above, notably a NH₂ group,
a —CONR′_{α}R_{β} group, R′_{α} and R_{β} being as defined above, notably —CONH₂,
a —NHCOR′_{α} group, R′_{α} being as defined above, and
a 2-pyridinyl group,
   one of the atoms among A₁, A₂ and A₃ representing N, and the two other atoms from A₁, A₂ and A₃ representing CH,
   the group Rᵢ being a hydrogen or an alkyl group comprising from 1 to 10 carbon atoms.

The “alkenyl” radicals represent hydrocarbon radicals with a straight or linear chain, and comprise one or several ethylenic unsaturations. When they comprise a single double bond, they may typically be represented by the formula CₙH₂ₙ, n representing the number of carbon atoms. Among alkenyl radicals, mention may notably be made of allyl or vinyl radicals.

The “alkynyl” radicals represent hydrocarbon radicals with a straight or linear chain and comprising one or several acetylenic unsaturations. When they comprise a single triple bond, they may typically be represented by the formula CₙH₂ₙ₋₂, n representing the number of carbon atoms. Among alkynyl radicals, mention may notably be made of acetylene.

Among the aryl groups, mention may be made of:

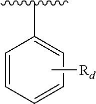

R_{d} being as defined above, and preferably being selected from the group consisting of: OCH₂OCH₃, OH, NO₂ and NR′_{α}R_{β}, R′_{α} and R_{β} being as defined above.

Among the aryl groups, mention may also be made of:

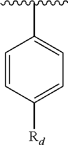

R_{d} being as defined above, and being preferably as selected from the group consisting of: CHO, SH, CN, OH, CF₃, CH₂OH and SO₂Me.

Among the heteroaryl groups, mention may be made of:

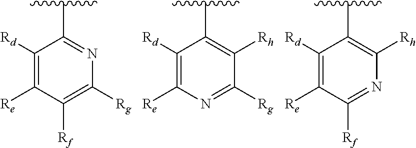

R_{d}, Rₑ, R_{f}, R_{g} and Rₕ being selected, independently of each other from the group consisting of the following substituents: a hydrogen, a halogen, notably Br, Cl or F, and an alkyl group comprising from 1 to 10 carbon atoms, and preferably being a methyl group.

Among the latter, mention may notably be made of the following groups:

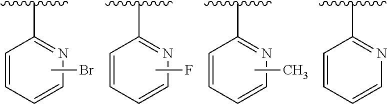

Among heteroaryl groups, mention may also be made of:

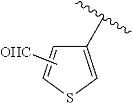

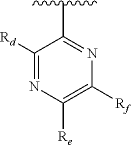

Other heteroaryl groups are the following:
R_{d}, Rₑ and R_{f} being selected independently of each other, in the group consisting of the following substituents: a hydrogen, a halogen notably Br, Cl or F, and an alkyl group comprising from 1 to 10 carbon atoms and preferably being a methyl group.

Among the latter, the following group may be mentioned:

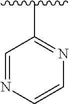 [SMILES: *c1cnccn1]

As an aryl group, mention may notably be made of the group fitting the following formula:

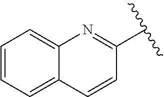 [SMILES: *c1ccc2ccccc2n1]

The “alkoxy” radicals according to the present invention are radicals of formula —O-alkyl, the alkyl group being as defined earlier.

The term of “alkylthio” designates a group —S-alkyl, the alkyl group being as defined above.

The term of “alkylamino” designates a group —NH-alkyl, the alkyl group being as defined above.

The term of “alkylcarbonyl” designates a group —CO-alkyl, the alkyl group being as defined above.

The term of “alkylcarboxyl” designates a group —COO-alkyl, the alkyl group being as defined above.

The term of “alkylsulfonyl” designates a group —SO₂-alkyl, the alkyl group being as defined above.

Among halogens, mention is more particularly made of fluorine, chlorine, bromine and iodine atoms.

The term of “aryloxy” designates a group —O-aryl, the aryl group being as defined above.

The term of “arylalkoxy” designates a group aryl-alkoxy-, the aryl and alkoxy groups being as defined above.

The term of “carboxyalkyl” designates a group HOOC-alkyl-, the alkyl group being as defined above. As an example of carboxyalkyl groups, mention may notably be made of carboxymethyl or carboxyethyl.

When an alkyl radical is substituted with an aryl group, this is referred to as an “arylalkyl” or “aralkyl” radical. The “arylalkyl” or “aralkyl” radicals are aryl-alkyl-radicals, the aryl and alkyl groups being as defined above. Among arylalkyl radicals, mention may notably be made of the benzyl or phenethyl radical. These arylalkyl groups may be substituted with one or several substituents. Among these substituents, mention may be made of the following groups: amino, hydroxy, thio, halogen, carboxyl, alkyl, alkoxy, alkylthio, alkylcarbonyl, alkylcarboxyl, alkylamino, aryloxy, arylalkoxy, cyano, trifluoromethyl, alkylsulfonyl carboxy or carboxyalkyl.

The expression “pharmaceutically acceptable salts” refers to relatively non-toxic inorganic and organic acid addition salts and base addition salts of the compounds of the present invention. These salts may be prepared in situ during the final isolation and purification of the compounds. In particular, the acid addition salts may be prepared by reacting separately the purified compound in its purified form with an organic or inorganic acid and by isolating the thereby formed salts. Among examples of acid addition salts, are found hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamates, malonates, salicylates, propionates, methylenebis-b-hydroxynaphthoates, gentisic acid, isethionates, di-p-toluoyltartrates, methanesulfonates, ethanesulfonates, benzenesulfonates, p-toluenesulfonates, cyclohexylsulfamates and quinates-laurylsulfonate salts, and the like (See for example S. M. Berge et al. <<Pharmaceutical Salts>< J. Pharm. Sci, 66: p. 1-19 (1977)). Acid addition salts may also be prepared by separately reacting the purified compound in its acid form with an organic or inorganic base and by isolating the thereby formed salts. Acid addition salts comprise amine and metal salts. Suitable metal salts comprise sodium, potassium, calcium, barium, zinc, magnesium and aluminum salts. Sodium and potassium salts are preferred. Suitable inorganic base addition salts are prepared from metal bases which comprise sodium hydride, sodium hydroxide, potassium hydroxide, calcium hydroxide, aluminum hydroxide, lithium hydroxide, magnesium hydroxide, zinc hydroxide. Suitable amine base addition salts are prepared from amines which have sufficient alkalinity in order to form a stable salt, and preferably comprise amines which are often used in medicinal chemistry because of their low toxicity and of their acceptance for medical use: ammonia, ethylenediamine, N-methyl-glucamine, lysine, arginine, ornithine, choline, N,N′-dibenzylethylenediamine, chloroprocaine, diethanolamine, procaine, N-benzyl-phenethylamine, diethylamine, piperazine, tris(hydroxymethyl)-aminomethane, tetramethyl-ammonium hydroxide, triethylamine, dibenzylamine, ephenamine, dehydroabietylamine, N-ethylpiperidine, benzylamine, tetra-methylammonium, tetra-ethylammonium, methylamine, dimethylamine, trimethylamine, ethylamine, basic amino acids, for example lysine and arginine and dicyclohexylamine, and the like.

The invention also relates to the tautomeric, enantiomeric, diastereoisomeric, epimeric forms and to the organic or mineral salts of the compounds of general formula (I).

The present invention also relates to the compounds of general formula (I) wherein:
R₁ is selected from the group consisting of:
   hydrogen,
   halogens,
   (hetero)aryls comprising from 5 to 30 carbon atoms optionally substituted,
   groups —NRₐR_{b}, Rₐ and R_{b} being independently selected from the group consisting of hydrogen, alkyl groups comprising from 1 to 10 carbon atoms, aryl groups comprising from 5 to 30 carbon atoms and arylalkyl groups comprising from 6 to 30 carbon atoms, said alkyls, aryls and arylalkyls being optionally substituted, it being understood that the group —NRₐR_{b} does not represent a —NH₂ group.

The present invention also relates to the compounds of general formula (I) wherein:
R₁ is selected from the group consisting of:
   hydrogen,
   halogens,
   (hetero)aryls comprising from 5 to 30 carbon atoms, optionally substituted.

According to an embodiment, the present invention relates to the compounds of the formula (I) as defined above, wherein R₁ represents a phenyl group, optionally substituted.

According to another embodiment, the present invention relates to the compounds of formula (I) as defined above, wherein R₁ represents a non-substituted phenyl group.

According to another embodiment, the present invention relates to the compounds of formula (I) as defined above, wherein R₁ represents a phenyl group substituted with a substituent selected from the group consisting of OCH₂OCH₃, OH, NO₂ and NR′_{α}R_{β}, R′_{α} and R_{β} being as defined above.

Preferably, in formula (I), R₂ represents a phenyl group optionally substituted, preferably with a group OR_{α}, R_{α} representing H or an alkyl group comprising from 1 to 10 carbon atoms.

When R₂ is a substituted phenyl group, it may then comprise one or more substituents indiscriminately located in the ortho, meta or para position, notably OH or alkoxy.

A family according to the present invention consists of compounds of formula (I) as defined above, wherein R₁ and R₂ represent a phenyl group, optionally substituted, and R₃ represents a halogen atom.

Thus, a compound of the invention fits the following formula:

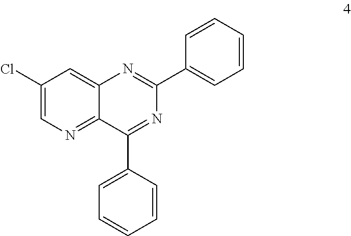 [SMILES: Clc1cnc2c(-c3ccccc3)nc(-c3ccccc3)nc2c1]

A family according to the present invention therefore consists of compounds of formula (I) as defined above, wherein R₁ is a phenyl group and R₂ represents a phenyl group, optionally substituted, preferably with a group OR_{α}, R_{α} being as defined above.

Among the compounds of the invention, mention may notably be made of the compounds of the following formula (I-a):

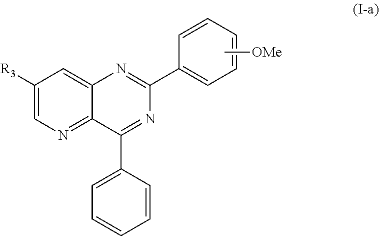

R₃ being as defined above for the formula (I)

The compounds of formula (I-a) are compounds of formula (I) wherein R₁ is a phenyl group and R₂ represents a phenyl group indiscriminately substituted in the ortho, meta or para position with a methoxy group.

Among the compounds of the invention, mention may notably be made of the compounds of the following formula (I-1):

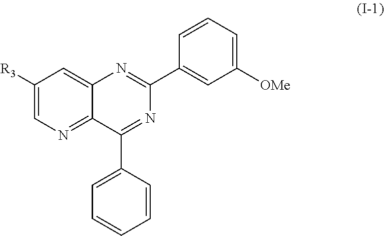

R₃ being as defined above in formula (I).

The compounds of formula (I-1) are compounds of formula (I) in which R₁ is a phenyl group and R₂ represents a phenyl group substituted in the meta position with a methoxy group.

Preferably, for the compounds of formula (I-1) and (I-a), R₃ is selected from the group consisting of:
Cl,
phenyl, optionally substituted, preferably in the para position, notably with a group OR_{α}, COR_{α} or SO₂R_{α}, R_{α} representing H or an alkyl group comprising from 1 to 10 carbon atoms, preferably methyl,
naphthyl, preferably 2-naphthyl,
furanyl, preferably 2-furanyl,
thiophenyl, preferably 2-thiophenyl,
pyridyl, preferably 3-pyridyl, and
benzo[b]thiophenyl, preferably 3-benzo[b]thiophenyl.

Thus, the present invention relates to the following compounds:

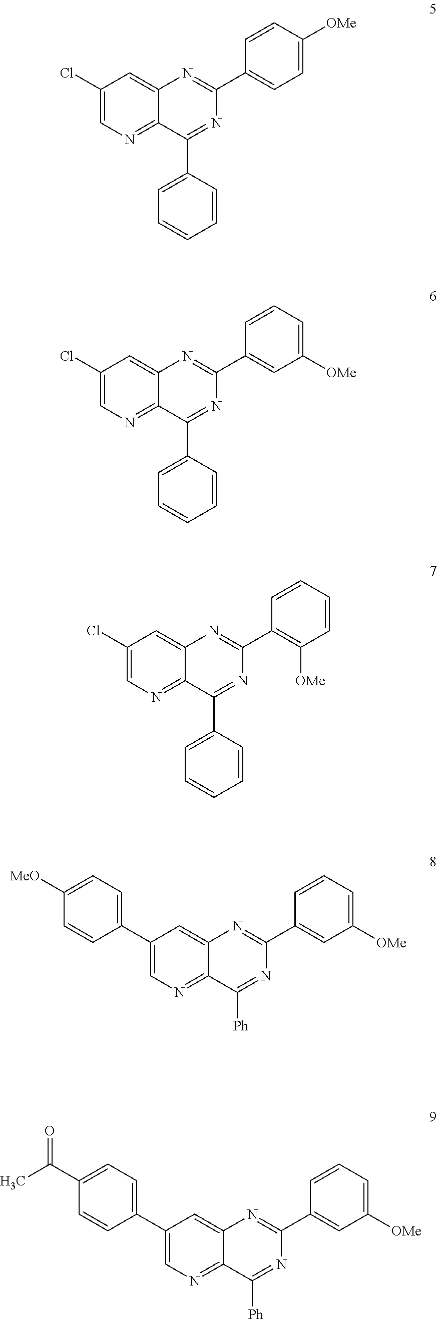 [SMILES: COc1ccc(-c2nc(-c3ccccc3)c3ncc(Cl)cc3n2)cc1 ; COc1cccc(-c2nc(-c3ccccc3)c3ncc(Cl)cc3n2)c1 ; COc1ccccc1-c1nc(-c2ccccc2)c2ncc(Cl)cc2n1 ; COc1ccc(-c2cnc3c(-c4ccccc4)nc(-c4cccc(OC)c4)nc3c2)cc1 ; COc1cccc(-c2nc(-c3ccccc3)c3ncc(-c4ccc(C(C)=O)cc4)cc3n2)c1]

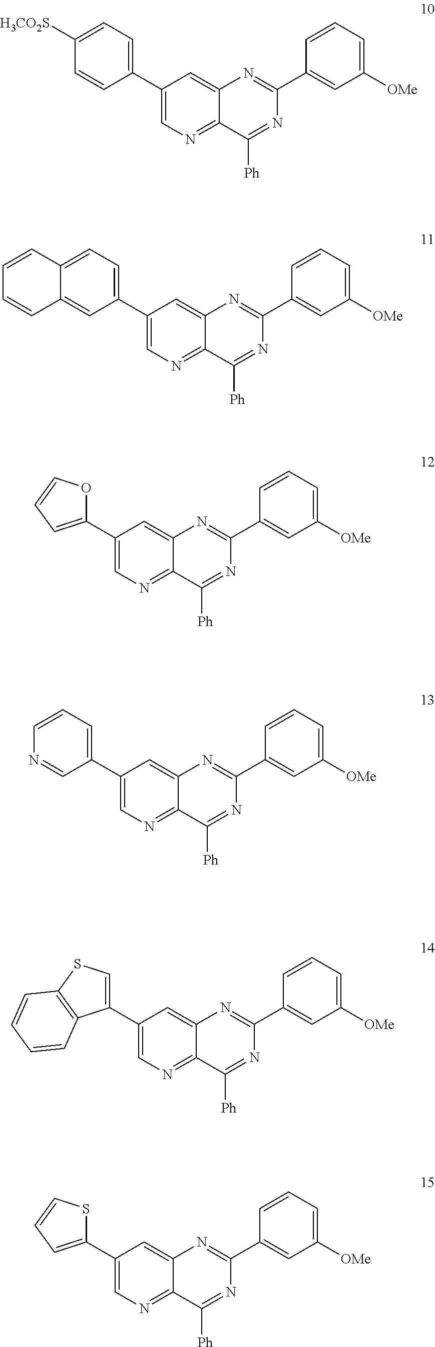

According to another embodiment, the present invention relates to compounds of formula (I) as defined above, wherein R₁ represents H.

A family according to the present invention therefore consists of formula (I) as defined above, wherein R₁ is H and R₂ represents a phenyl group, optionally substituted, preferably with a group OR_{α}, R_{α} being as defined above.

When R₂ is a substituted phenyl group, it may then comprise one or several substituents indiscriminately located in the ortho, meta or para position, notably OH or alkoxy.

In the aforementioned family, mention may also be made of a sub-family of compounds wherein R₃ represents a halogen, notably Cl, or a phenyl group, if necessary substituted, preferably with a group Cl or OR_{α}, R_{α} representing H or an alkyl group comprising from 1 to 10 carbon atoms.

Among the compounds of the invention, mention may notably be made of the compounds of the following formula (I-2):

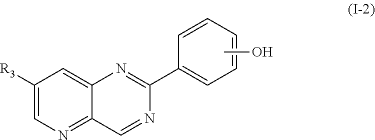

R₃ representing a halogen, notably Cl, or a phenyl group optionally substituted, preferably with a group Cl or OR_{α}, R_{α} representing H or an alkyl group comprising from 1 to 10 carbon atoms.

The compounds of formula (I-2) are compounds of formula (I) wherein R₁ is H, R₂ represents a phenyl group, indiscriminately substituted in the ortho, meta or para position, with a group OH and R₃ represents a halogen, notably Cl, or a phenyl group, optionally substituted, preferably with a group Cl or OR_{α}, R_{α} representing H or an alkyl group comprising from 1 to 10 carbon atoms.

Among these compounds of formula (I-2) mention may also be made of the compounds of the following formula (I-2-11:

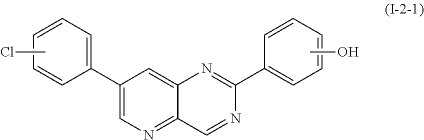

The compounds of formula (I-2-1) are compounds of formula (I) wherein R₁ is H, R₂ represents a substituted phenyl group, indiscriminately in the ortho, meta or para position, with an OH group, and R₃ represents a substituted phenyl group, indiscriminately in the ortho, meta or para position with Cl.

Thus, the present invention relates to the following particular compounds:

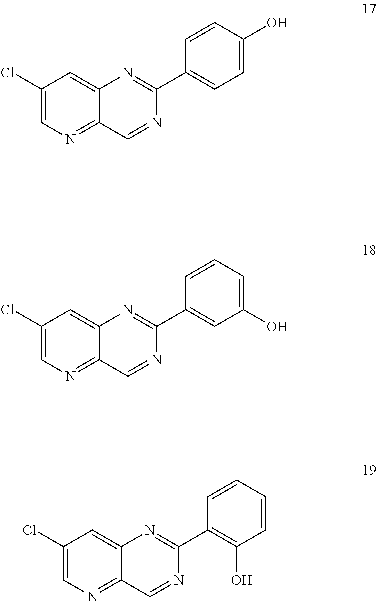 [SMILES: Oc1ccc(-c2ncc3ncc(Cl)cc3n2)cc1 ; Oc1cccc(-c2ncc3ncc(Cl)cc3n2)c1 ; Oc1ccccc1-c1ncc2ncc(Cl)cc2n1]

Among the compounds of formula (I-2), mention may also be made of the compounds of the following formula (I-2-2):

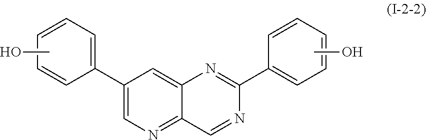

The compounds of formula (I-2-2) are compounds of formula (I) wherein R₁ is H, R₂ represents a phenyl group, indiscriminately substituted in the ortho, meta or para position, with OH, and R₃ represents a phenyl group, indiscriminately substituted in the ortho, meta or para position, with OH.

Thus, the present invention relates to the following particular compounds:

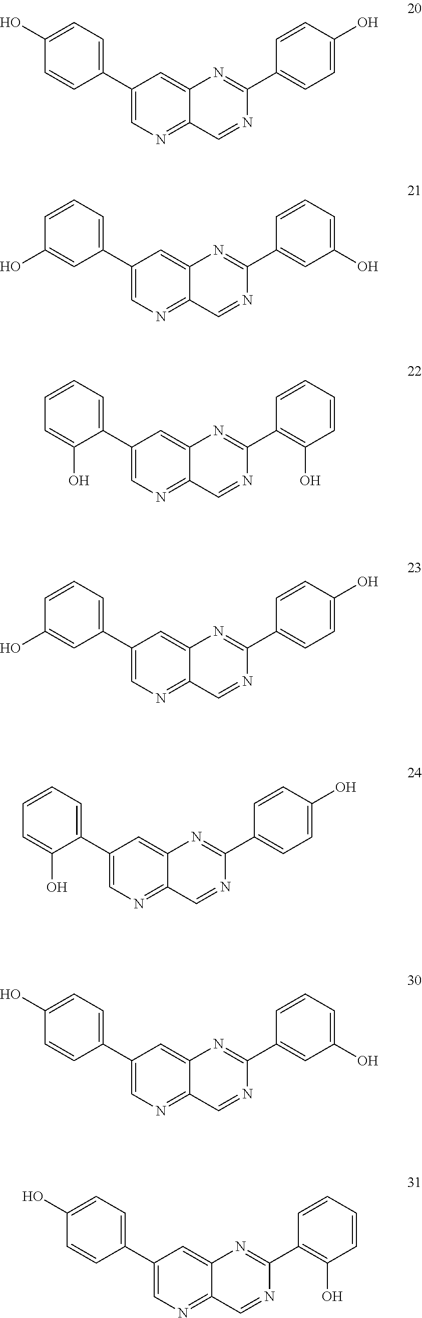

Among the compounds of formula (I-2), mention may also be made of the compounds of the following formula (I-2-3):

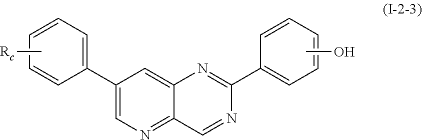

R_{c} being selected from the group consisting of CF₃, CN, CH₂OH, CHO, SO₂R_{α} and SR_{α}, R_{α} representing H or an alkyl group comprising from 1 to 10 carbon atoms.

The compounds of formula (I-2-3) are compounds of formula (I) wherein R₁ is H, R₂ represents a phenyl group, indiscriminately substituted in the ortho, meta or para position, with OH, and R₃ represents a phenyl group indiscriminately substituted in the ortho, meta or para position, with a group R_{c} as defined above.

Thus, the present invention relates to the following particular compounds:

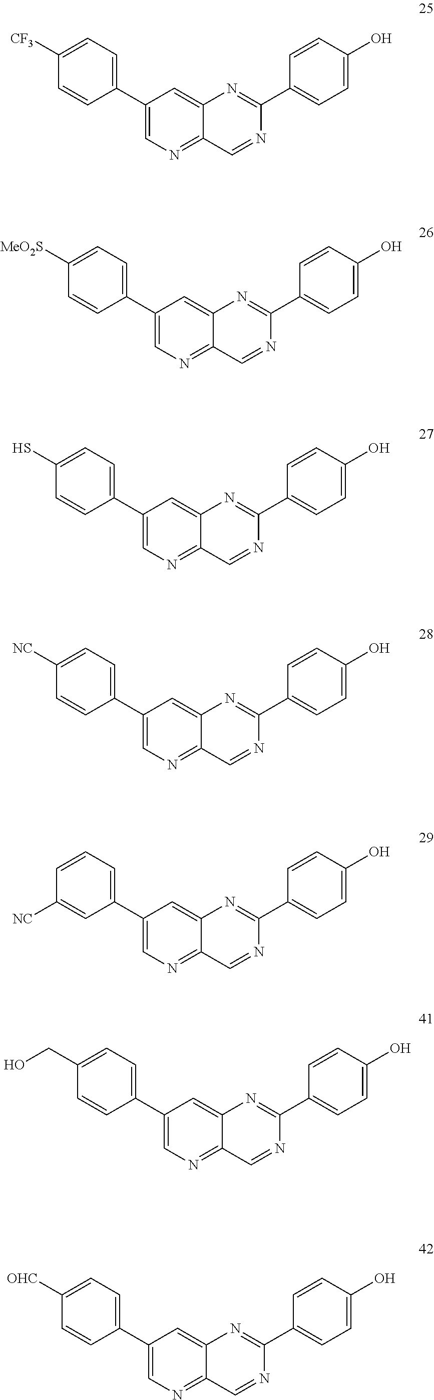

Among the compounds of the invention, mention may notably be made of the compounds of the following formula (I-3):

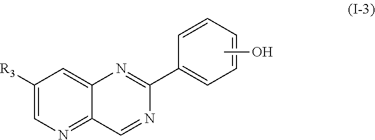

R₃ being as defined above in formula (I).

The compounds of formula (I-3) are compounds of formula (I) wherein R₁ is H and R₂ represents a phenyl group, indiscriminately substituted in the ortho, meta or para position, with an OH group.

Preferably, in formula (I-3), R₃ is selected from the group consisting of the following groups:
halogen, notably Cl,
furanyl, notably 2- or 3-furanyl,
thiophenyl, notably 2- or 3-thiophenyl, optionally substituted with one or several substituents notably CH₂OH or COOH,
pyridyl, notably 3- or 4-pyridyl, optionally substituted with one or several substituents notably OR_{α}, R_{α} representing H or an alkyl group comprising from 1 to 10 carbon atoms,
phenyl, optionally substituted with one or several substituents notably selected from CN or OR_{α}, R_{α} being as defined above,
benzothiazolyl, notably 2-benzothiazolyl, and
a group —NHR″_{b}, R″_{b} being selected from the group consisting of the following groups:
   phenyl, optionally substituted with one or several substituents notably selected from CN, CF₃, SO₂R_{α}, SR_{α} or OR_{α}, R_{α} being as defined above,
   pyridyl, notably 2-, 3- or 4-pyridyl, optionally substituted with one or several substituents, notably with an alkyl group comprising from 1 to 10 carbon atoms,
   pyrimidinyl, notably 2-pyrimidinyl,
   thiazolyl, notably 2-thiazolyl, optionally substituted with one or several substituents, notably with an alkyl group comprising from 1 to 10 carbon atoms, and
   an isoxazolyl, notably 3-isoxazolyl.

A class of compounds according to the present invention consists of compounds of formula (I-2-3) described above, wherein R_{c} represents a heteroaryl group.

Preferably, R_{c} is a heteroaryl group selected from the group formed by furanyl groups, notably 2- or 3-furanyl, thiophenyl, notably 2- or 3-thiophenyl, optionally substituted with one or several substituents, notably CH₂OH or COOH, and pyridyl, notably 3- or 4-pyridyl, optionally substituted with one or several substituents notably OR_{α}, R_{α} representing H or an alkyl group comprising from 1 to 10 carbon atoms.

Thus, the present invention relates to the following particular compounds:

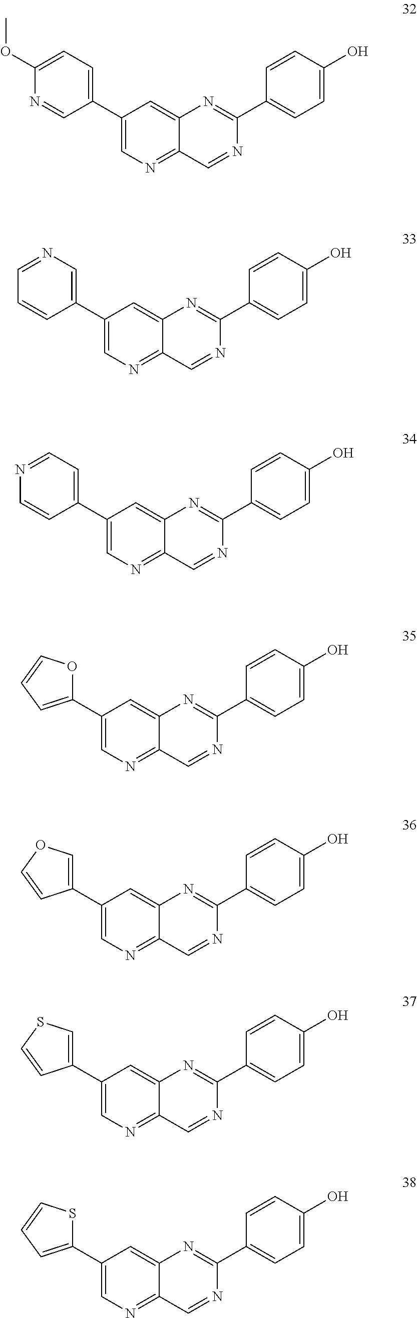

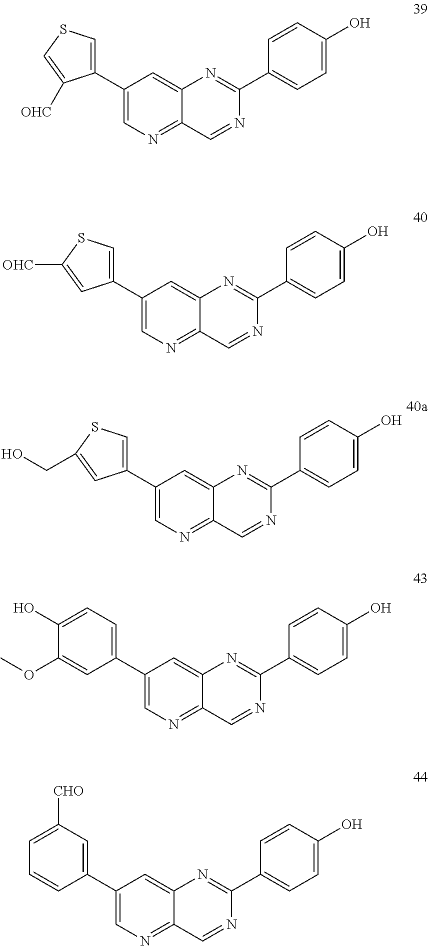 [SMILES: O=Cc1cscc1-c1cnc2cnc(-c3ccc(O)cc3)nc2c1 ; O=Cc1cc(-c2cnc3cnc(-c4ccc(O)cc4)nc3c2)cs1 ; OCc1cc(-c2cnc3cnc(-c4ccc(O)cc4)nc3c2)cs1 ; COc1cc(-c2cnc3cnc(-c4ccc(O)cc4)nc3c2)ccc1O ; O=Cc1cccc(-c2cnc3cnc(-c4ccc(O)cc4)nc3c2)c1]

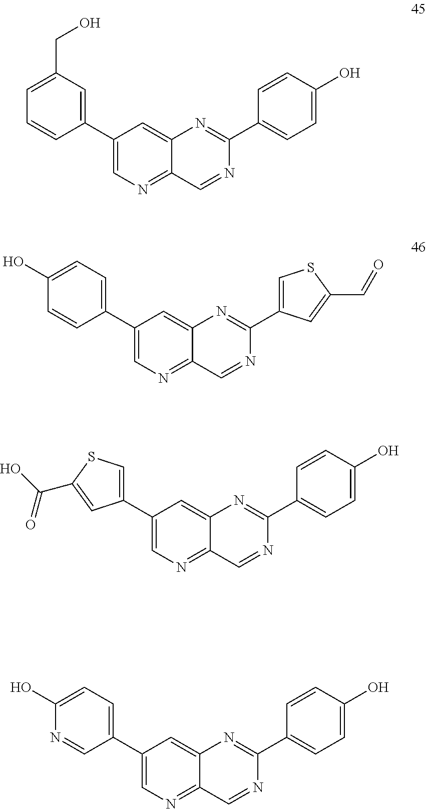 [SMILES: OCc1cccc(-c2cnc3cnc(-c4ccc(O)cc4)nc3c2)c1 ; O=Cc1cc(-c2ncc3ncc(-c4ccc(O)cc4)cc3n2)cs1 ; O=C(O)c1cc(-c2cnc3cnc(-c4ccc(O)cc4)nc3c2)cs1 ; Oc1ccc(-c2ncc3ncc(-c4ccc(O)nc4)cc3n2)cc1]

A class of compounds according to the present invention consists of compounds of formula (I-3) described above, wherein R₃ represents a group —NHR″_{b}, R″_{b} being as defined above. Preferably, R″_{b} is a (hetero)aryl group.

A class of compounds according to the present invention consists of compounds of the following formula (I-5):

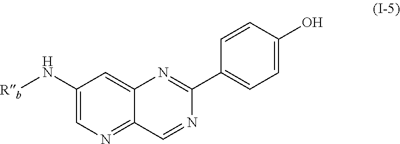

R″_{b} representing a (hetero)aryl group, preferably selected from the following groups:

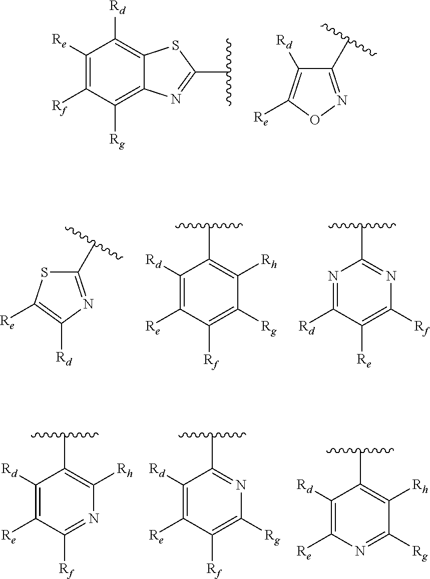

the groups R_{d}, Rₑ, R_{f}, R_{g} and Rₕ being as defined above.

Thus, the present invention relates to the particular compounds:

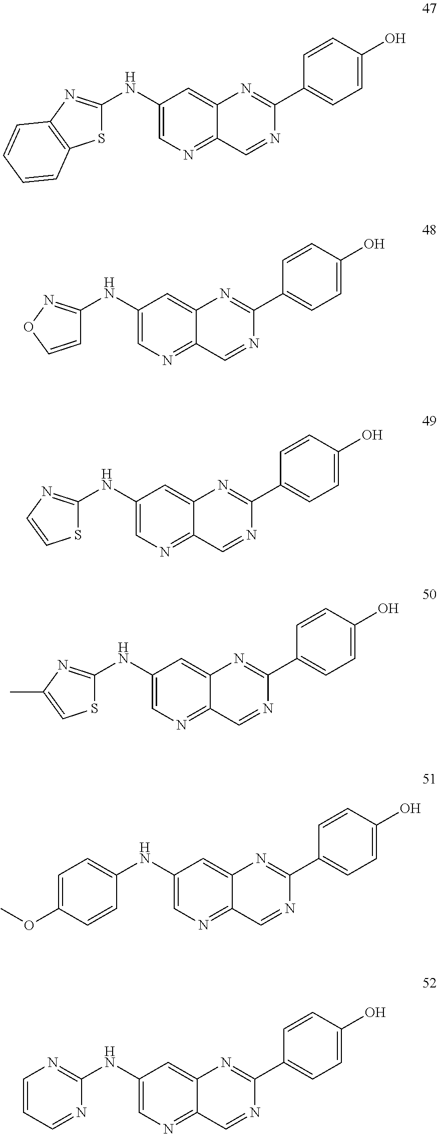 [SMILES: Oc1ccc(-c2ncc3ncc(Nc4nc5ccccc5s4)cc3n2)cc1 ; Oc1ccc(-c2ncc3ncc(Nc4ccon4)cc3n2)cc1 ; Oc1ccc(-c2ncc3ncc(Nc4nccs4)cc3n2)cc1 ; Cc1csc(Nc2cnc3cnc(-c4ccc(O)cc4)nc3c2)n1 ; COc1ccc(Nc2cnc3cnc(-c4ccc(O)cc4)nc3c2)cc1 ; Oc1ccc(-c2ncc3ncc(Nc4ncccn4)cc3n2)cc1]

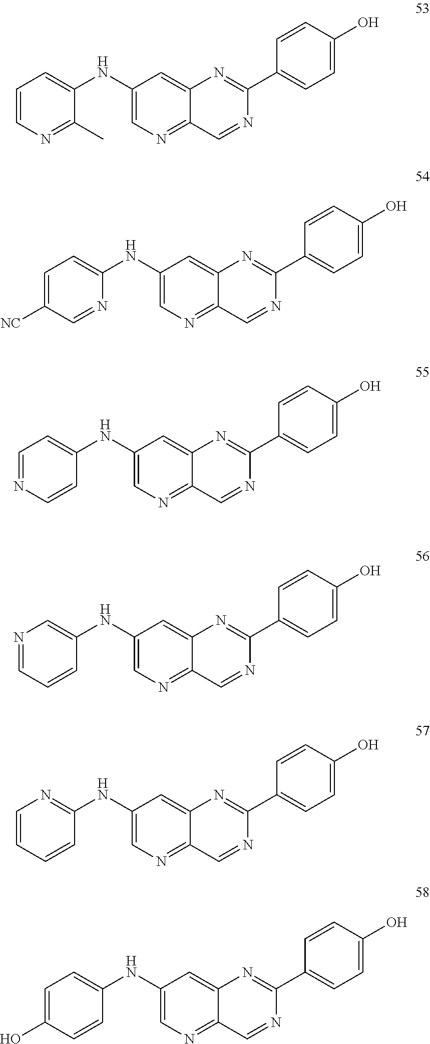 [SMILES: Cc1ncccc1Nc1cnc2cnc(-c3ccc(O)cc3)nc2c1 ; N#Cc1ccc(Nc2cnc3cnc(-c4ccc(O)cc4)nc3c2)nc1 ; Oc1ccc(-c2ncc3ncc(Nc4ccncc4)cc3n2)cc1 ; Oc1ccc(-c2ncc3ncc(Nc4cccnc4)cc3n2)cc1 ; Oc1ccc(-c2ncc3ncc(Nc4ccccn4)cc3n2)cc1 ; Oc1ccc(Nc2cnc3cnc(-c4ccc(O)cc4)nc3c2)cc1]

Among the compounds of the invention, mention may notably be made of the compounds of the following formula (I-c):

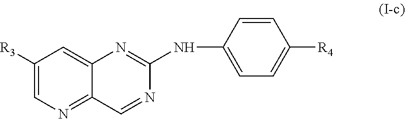

R₃ being as defined above, and

R₄ being selected from the group consisting of OH, alkoxy and amine groups.

Preferably, in the formula (I-c), R₃ is a halogen, preferably Cl, or a phenyl group substituted with an OH group (indiscriminately in the ortho, meta or para position).

A family of compounds according to the invention consists of compounds of formula (I-c) wherein R₄ is OH.

Thus, the present invention relates to the following particular compounds:

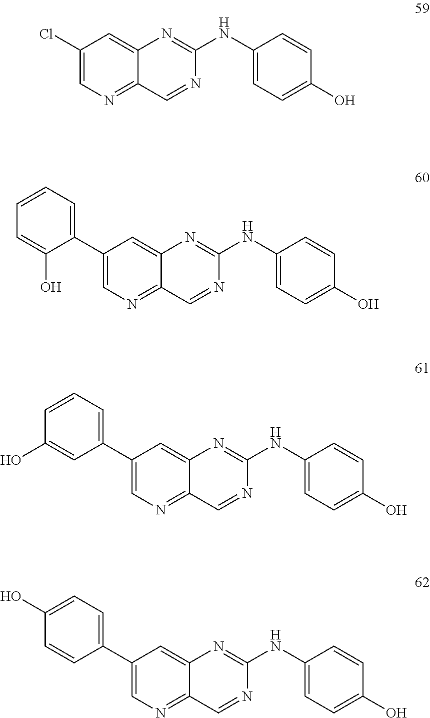 [SMILES: Oc1ccc(Nc2ncc3ncc(Cl)cc3n2)cc1 ; Oc1ccc(Nc2ncc3ncc(-c4ccccc4O)cc3n2)cc1 ; Oc1ccc(Nc2ncc3ncc(-c4cccc(O)c4)cc3n2)cc1 ; Oc1ccc(Nc2ncc3ncc(-c4ccc(O)cc4)cc3n2)cc1]

A family of compounds according to the invention consists of compounds of the following formula (I-c-1):

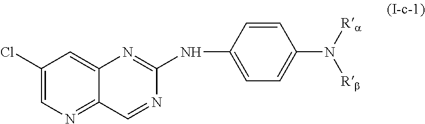

R′_{α} and R_{β} being as defined above and preferably representing a methyl group.

A family of compounds according to the invention consists of compounds of the following formula (I-d):

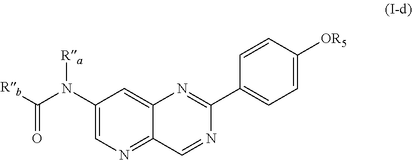

R″ₐ and R″_{b} being as defined above, and R₅ representing H or a group —(CH₂)ₙ—O—R′_{α} and n being as defined above, and preferably R₅ representing H or a —CH₂OCH₃ group.

According to a particular embodiment, N(R″ₐ)COR″_{b} forms a heterocycle with 5 or 6 atoms selected from:

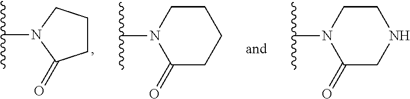

Thus, the present invention relates to the following particular compounds:

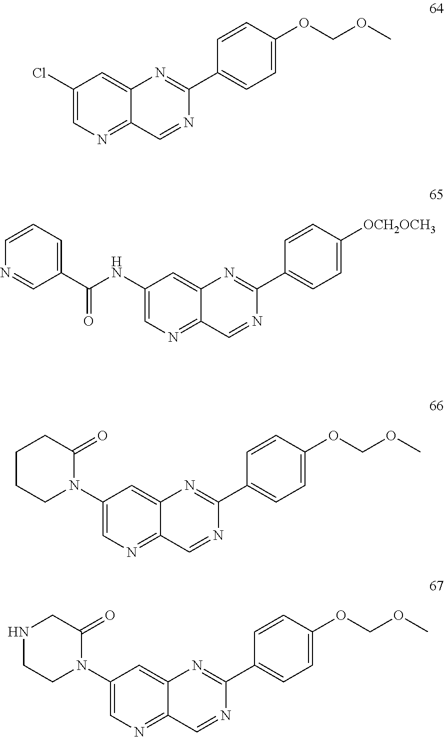

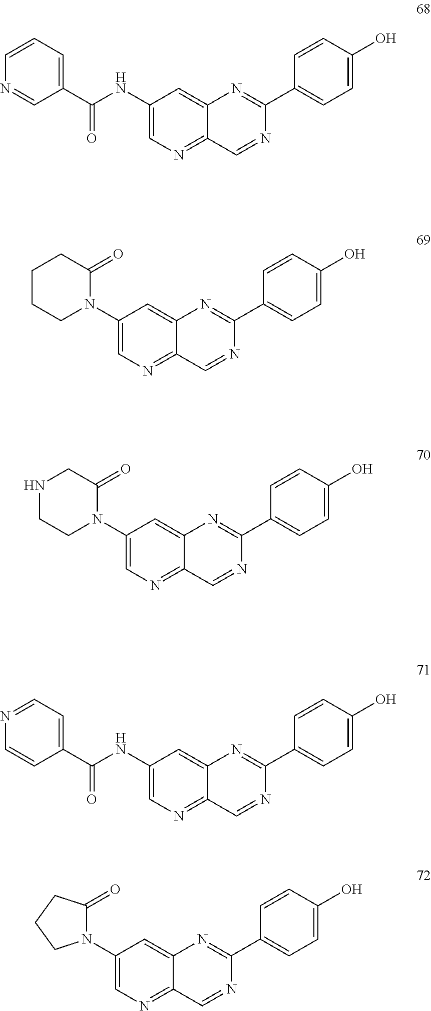 [SMILES: O=C(Nc1cnc2cnc(-c3ccc(O)cc3)nc2c1)c1cccnc1 ; O=C1CCCCN1c1cnc2cnc(-c3ccc(O)cc3)nc2c1 ; O=C1CNCCN1c1cnc2cnc(-c3ccc(O)cc3)nc2c1 ; O=C(Nc1cnc2cnc(-c3ccc(O)cc3)nc2c1)c1ccncc1 ; O=C1CCCN1c1cnc2cnc(-c3ccc(O)cc3)nc2c1]

A family of compounds according to the invention consists of compounds of the following formula (I-e):

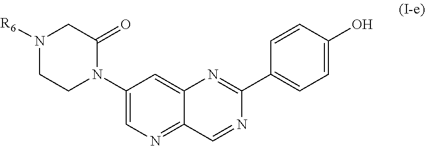

R₆ being selected from the group consisting of: alkyl, notably methyl, aralkyl, notably benzyl, —CH₂-HetAr, notably —CH₂-(3- or 4-pyridine), alkylcarbonyl, notably

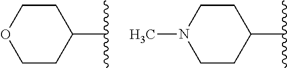

COCH₃, —CO-HetAr, notably —CO-(2-pyridine), —N(R″ₐ)CON(R″_{b}), R″ₐ and R″_{b} being as defined above, and

According to another embodiment, the present invention relates to compounds of formula (I) as defined above, wherein R₁ represents NHBn, Bn representing a benzyl group (—CH₂Ph).

Preferably, the compounds of the invention are compounds of formula (I) wherein R₂ represents a —NH—(CH₂)₂—OH group.

Among the compounds of the invention mention may notably be made of the compounds of the following formula (I-4):

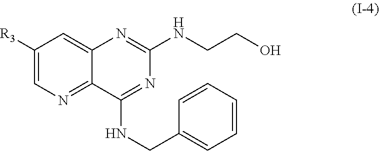

R₃ being as defined above for formula (I).

The compounds of formula (I-4) are compounds of formula (I) wherein R₁ represents NHBn and R₂ represents a —NH—(CH₂)₂—OH group.

Preferably, in formula (I-4), R₃ is a halogen, notably Cl, and a group —NHR″_{b}, R″_{b} being an aryl or heteroaryl group comprising from 6 to 30 carbon atoms, optionally substituted.

Another family of compounds of the invention consists of compounds of formula (I-4) as defined above, wherein R₃ is a —NHR″_{b}, R″_{b} representing a phenyl group, optionally substituted with one or several substituents, indiscriminately in the ortho, meta or para position, selected from the group consisting of OR_{α} or COR_{α}, R_{α} representing H or an alkyl group comprising from 1 to 10 carbon atoms.

Another family of compounds according to the invention consists of compounds of formula (I-4) as defined above, wherein R₃ is a —NHRₐ group, Rₐ representing a heteroaryl group selected from the group consisting of the following groups:

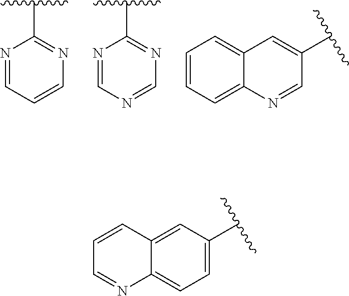

these groups may optionally be substituted with one or several substituents as defined above.

Thus the present invention relates to the following particular compounds:

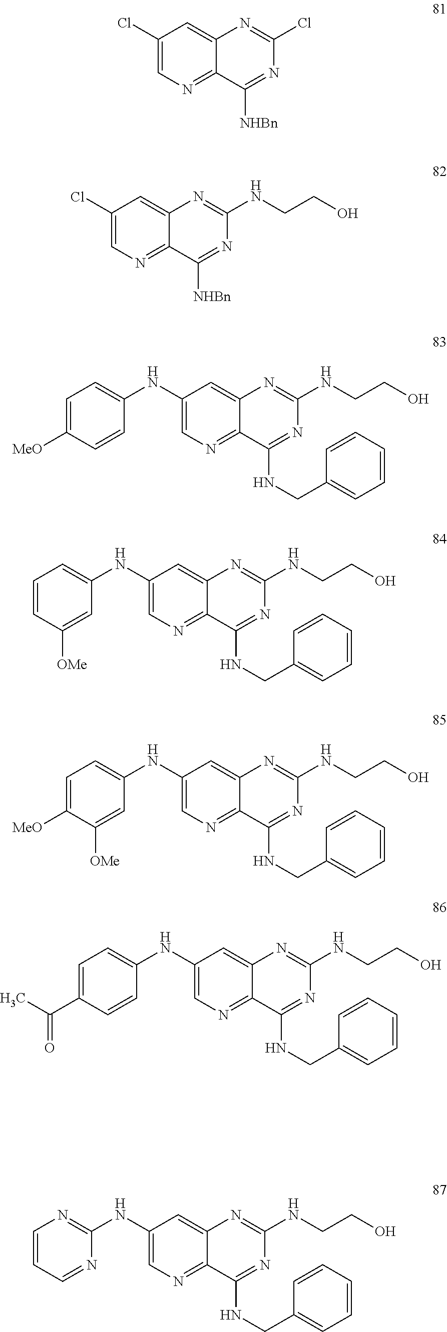

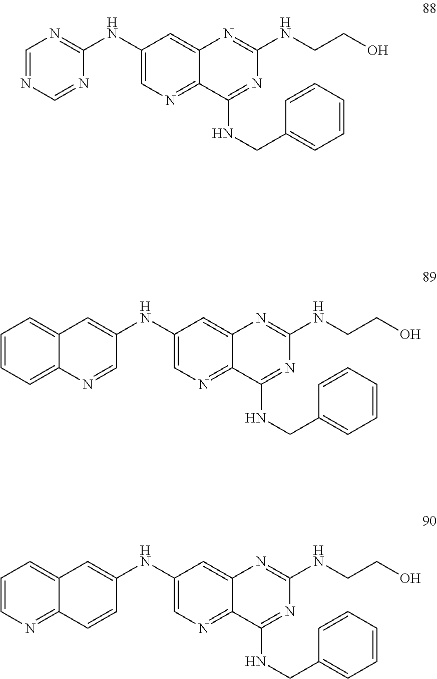 [SMILES: OCCNc1nc(NCc2ccccc2)c2ncc(Nc3ncncn3)cc2n1 ; OCCNc1nc(NCc2ccccc2)c2ncc(Nc3cnc4ccccc4c3)cc2n1 ; OCCNc1nc(NCc2ccccc2)c2ncc(Nc3ccc4ncccc4c3)cc2n1]

The present invention also relates to a pharmaceutical composition comprising a compound of formula (I) as defined above, or any compound as mentioned above, in combination with a pharmaceutically acceptable carrier.

The present invention therefore relates to a compound as defined above of formula (I) for its use as a drug.

The pharmaceutical compositions according to the invention may appear in forms intended for administration via a parenteral, oral, rectal, permucosal or percutaneous route.

The pharmaceutical compositions including these compounds of general formula (I) will therefore appear in the form of solutes or injectable suspensions or multi-dosed vials, in the form of exposed or coated tablets, dragees, capsules, gelatine capsules, pills, cachets, powders, suppositories or rectal capsules, solutions or suspensions, for percutaneous use in a polar solvant, for permucosal use.

The excipients which are suitable for such administration are derivatives of cellulose or of microcrystalline cellulose, earth alkaline carbonates, magnesium phosphate, starches, modified starches, lactose for solid forms.

For rectal use, cocoa butter or polyethylenglycol stearates are preferred excipients.

For parenteral use, water, aqueous solutes, saline, isotonic solutes are the most conveniently used carriers.

The dosage may vary within wide limits (0.5 mg to 1,000 mg) depending on the therapeutic indication and on the administration route, as well as on the age and on the weight of the subject.

The present invention also relates to a compound as defined above of formula (I), or any compound as mentioned above, for its use as an inhibitor of CDK1, CDK5, GSK3 and/or DYRK1A kinases.

The present invention also relates to a compound as defined above of formula (I), or any compound as mentioned above, for its use within the scope of treating or preventing diseases related to deregulation of the CDK1, CDK5, GSK3 and/or DYRK1A kinases.

More particularly, said diseases are selected from the group consisting of cancers, Alzheimer's disease, Parkinson's disease, brain traumas, cerebrovascular strokes, renal polycystoses, amyotrophic lateral scleroses, viral infections, auto-immune diseases, neurodegenerative disorders, psoriasis, asthma, atopical dermatitises, trisomia 21 and glomerulonephrites.

The present invention also relates to the use of the compounds of the invention as defined above, for preparing a drug intended for treating or preventing diseases related to deregulation of the CDK1, CDK5, GSK3 and/or DYRK1A kinases, and more particularly treating and preventing the aforementioned diseases.

The present invention also relates to a method for preparing an intermediate synthesis compound of the following formula (2):

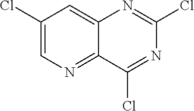 [SMILES: Clc1cnc2c(Cl)nc(Cl)nc2c1]

said method comprising a trichorination step, notably in the presence of POCl₃/PCl₅, with microwave irradiation of the compound (1) of the following formula:

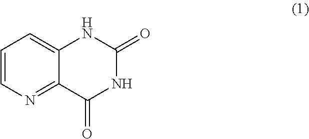 [SMILES: O=c1[nH]c(=O)c2ncccc2[nH]1]

and optionally a step for isolating said compound (2):

The aforementioned trichlorination step is preferably carried out at 160° C. for about 2 hours.

The present invention also relates to a method for preparing a compound of the following formula (I-1-1):

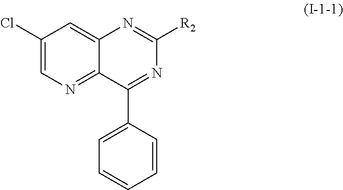

R₂ being an aryl group as defined above, and notably a phenyl group, either substituted or not,

said method comprises the following steps:

a) a step for Suzuki coupling (regioselective in position 4) of the compound of formula (2) as defined above,

in the presence of the compound PhB(OH)₂, in order to obtain the intermediate compound (3):

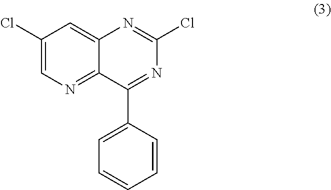 [SMILES: Clc1cnc2c(-c3ccccc3)nc(Cl)nc2c1]

b) a step for Suzuki coupling (regioselective in position 2) of the aforementioned compound (3) in the presence of the compound R₂B(OH)₂, R₂ being as defined above, in order to obtain the aforementioned compound (I-1-1), and

c) optionally a step for isolating the compound (I-1-1).

Preferably, the aforementioned step a) is carried out in the presence of K₂CO₃. It is also carried out in the presence of a catalyst such as Pd(PPh₃)₄, in a solvent like toluene, at 100° C. for 2 hours.

Preferably, the aforementioned step b) is carried out in the presence of Na₂CO₃. It is also carried out in the presence of a catalyst such as Pd(PPh₃)₄, in a solvent such as a toluene/ethanol mixture, at 100° C.

The present invention also relates to a method for preparing a compound of the following formula (I-1-2):

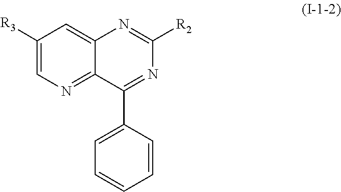

R₂ being an aryl group as defined above, and R₃ being an aryl or heteroaryl group as defined above,

said method comprising the following steps:

a) a step for Suzuki coupling of the compound of formula (I-1-1) as defined above, in the presence of the compound R₃B(OH)₂, R₃ being as defined above, for obtaining the aforementioned compound (I-1-2),

b) and optionally a step for isolating the compound (I-1-2).

Preferably, the aforementioned step a) is carried out in the presence of K₂CO₃. It is also carried out in the presence of a catalyst such as Pd(PPh₃)₄, in a solvent such as a toluene/ethanol mixture at 150° C. This step is preferably carried out with microwave irradiation for 5 to 15 minutes.

The present invention also relates to a method for preparing a compound of the following formula (I-3-1):

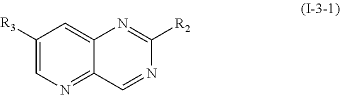

R₂ being an aryl group, if necessary substituted, as defined above, and R₃ being Cl or an aryl group or as defined above for R₂,

said method comprising the following steps:

a) a step for reacting the compound (2) as defined above in the presence of Bu₃SnH and of Pd(PPh₃)₄, in order to obtain the compound of the following formula (16):

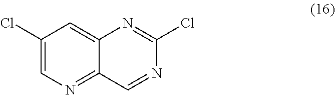 [SMILES: Clc1cnc2cnc(Cl)nc2c1]

b) a step for reacting the aforementioned compound (16) with a compound of formula R₂B(OH)₂, R₂ being as defined above, in order to obtain the aformementioned compound (I-3-1),

c) and optionally a step for isolating the compound (I-3-1).

Step a) is preferably carried out in the presence of the catalyst Pd(PPh₃)₄, in a solvent such as toluene, at 100° C. for about 10 minutes.

Step b) is preferably carried out in the presence of the catalyst Pd(PPh₃)₄, in a solvent such as a toluene/ethanol mixture.

When step b) is carried out in the presence of Na₂CO₃ and at 100° C., a compound of the aforementioned formula (I-3-1) is obtained, wherein R₃ is Cl.

When step b) is carried out in the presence of K₂CO₃ and at 150° C. with microwave irradiation, a compound of the aforementioned formula (I-3-1) is obtained wherein R₃ is an aryl group either substituted or not, identical with R₂.

The present invention also relates to a method for preparing a compound of formula (I-3) as defined above, comprising a step for reacting a compound of the following formula (I-3-2):

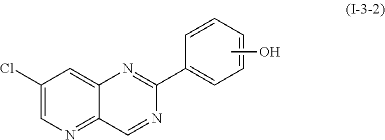

with a compound R₃B(OH)₂, R₃ being as defined above, and preferably being a phenyl group as defined above,

this step being optionally followed by a step for isolating the aforementioned compound (I-3).

Preferably, the step for reacting the compound (I-3-2) with the compound R₃B(OH)₂ is carried out in the presence of K₂CO₃ and of a catalyst such as Pd(PPh₃)₄, in a solvent such as a toluene/ethanol mixture at 150° C. This step is preferably carried out with microwave irradiation for 5 to 15 minutes.

The present invention also relates to a method for preparing a compound of formula (I-5) as defined above, comprising a step for amination of the aforementioned compound (17) with a compound R″_{b}NH₂, R″_{b} being as defined above.

This amination step is preferably carried out in the presence of K₂CO₃ and of the catalyst Pd(OAc)₂ and of xantphos in 1,4-dioxane with microwave irradiation at 140° C.

The present invention also relates to a method for preparing the compound (59) as defined above, comprising a step for amination of the aforementioned compound (16) with the amine (p-OH)C₄H₆—NH₂.

This amination step is preferably carried out in refluxing 1,4-dioxane for 24 hours.

The present invention also relates to a method for preparing a compound of formula (I-c) as defined above, comprising the reaction of the compound (59) with a compound R₃B(OH)₂, R₃ being as defined above, and preferably being a phenyl group as defined above.

This reaction step is preferably carried out in the presence of K₂CO₃ and of a catalyst such as Pd(PPh₃)₄, in a solvent such as a toluene/ethanol mixture, at 150° C. This step is preferably carried out with microwave irradiation for 5 to 15 minutes.

The present invention also relates to a method for preparing a compound of formula (I-d) as defined above, wherein R₅ is H, comprising a step for amination of the aforementioned compound (17) with a compound HN(R″ₐ)COR″_{b}, R″ₐ and R″_{b} being as defined above.

The present invention also relates to a method for preparing a compound of formula (I-d) as defined above, wherein R₅ is —(CH₂)ₙ—O—R_{α}, R′_{α} and n being as defined above, comprising the reaction of the compound (17) with a compound of formula Cl—(CH₂)ₙ—O—R′_{α}, preferably in the presence of K₂CO₃ and in acetone. This reaction is notably carried out at 0° C. at room temperature for 16 hours.

The present invention also relates to a method for preparing a compound of formula (I-4) as defined above, comprising an amination step with a compound R₃NH₂, wherein R₃ is preferably aryl or heteroaryl, for the compound of the following formula (82):

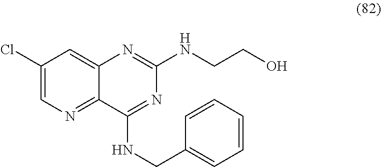 [SMILES: OCCNc1nc(NCc2ccccc2)c2ncc(Cl)cc2n1]

this amination step being optionally followed by a step for isolating the aforementioned compound (I-4).

The aforementioned amination step is preferably carried out in the presence of K₂CO₃ and of the catalyst Pd(OAc)₂ and of xantphos in 1,4-dioxane with microwave irradiation at 140° C.

The present invention also relates to a method for preparing as defined above, wherein the compound (82) is obtained according to the method comprising the following steps:

a) a step for triamination of the compound (2) as defined above with the compound BnNH₂ in order to obtain a compound of the following formula (81):

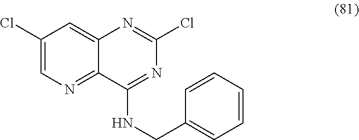 [SMILES: Clc1cnc2c(NCc3ccccc3)nc(Cl)nc2c1]

b) a step for amination of the aforementioned compound (81) with the compound H₂C₂H₄OH in order to obtain the compound (82),

c) and optionally a step for isolating the compound (82).

Step a) is notably carried out in the presence of triethylamine in tetrahydrofurane (THF) at room temperature for 4 hours.

Step b) is notably carried out in the presence of triethylamine in refluxing 1,4-dioxane for 12 hours.

The present invention also relates to a method for preparing a compound of formula (I-4) as defined above, R₃ preferably representing an amino, aryl or heteroaryl group, optionally substituted, comprising the following steps carried out sequentially or “one-pot”:

a) a step for triamination of the compound (2) as defined above with the compound BnNH₂ in order to obtain a compound of formula (81) as defined above;

b) a step for amination of the aforementioned compound (81) with the compound H₂C₂H₄OH in order to obtain the compound (82) as defined above;

c) and an amination step with a compound R₃NH₂ for the aforementioned compound (82), this amination step being optionally followed by a step for isolating the aforementioned compound (I-4).

Step a) is notably carried out in the presence of triethylamine in 1,4-dioxane at room temperature for 5 minutes.

Step b) is notably carried out with microwave irradiation at 140° C. for 1 hour.

Step c) is preferably carried out in the presence of K₂CO₃ and of the catalyst Pd(OAc)₂ and of xantphos with microwave irradiation at 140° C.

### EXPERIMENTAL PART

### 1. Preparation of the Compounds of the Invention

Preparation of the Syntheses Intermediate (2):

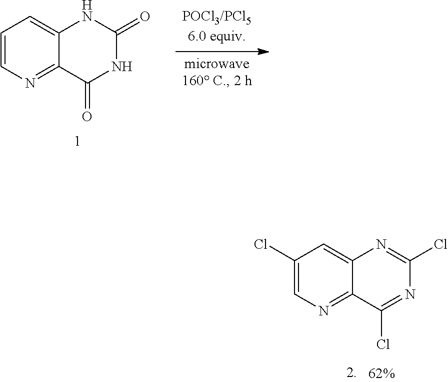 [SMILES: O=c1[nH]c(=O)c2ncccc2[nH]1 ; Clc1cnc2c(Cl)nc(Cl)nc2c1]

### 2,4,7-Trichloropyrido[3,2-d]pyrimidine (2)

In a 20 mL vial, 1.0 g (6.13 mmol, 1 equiv.) of 1H,3H-pyrido[3,2-c]pyrimidine-2,4-dione 1 is in suspension in 10 mL of phosphorus oxychloride and 7.65 g (36.7 mmol, 6.0 equiv.) of phosphorus pentachloride (PCl_{S}). The whole is heated with microwave irradiations to 160° C. After 2 hours of reaction, the POCl₃ excess is evaporated under reduced pressure. The obtained residue is brought to 0° C. by means of an ice bath and solubilized in dichloromethane, the mixture is poured in a water/ice mixture without any basification. After returning to room temperature, the aqueous phase is extracted with dichloromethane. The organic phase is then dried on MgSO₄, filtered, and then concentrated under reduced pressure. The thereby obtained residue is chromatographed on silica gel (petroleum ether/CH₂Cl₂, 40/60) in order to obtain white solid with a yield of 62%. MP: 165-166° C.; IR (ATR, Diamond, cm⁻¹) ν: 3048, 2167, 1579, 1531, 1430, 1324, 1253, 1136, 1001, 872; ¹H NMR (400 MHz, CDCl₃) δ: 8.31 (d, 1H, J=2.2 Hz, H₈), 9.03 (d, 1H, J=2.2 Hz, H₆); ¹³C NMR (100 MHz, CDCl₃) δ: 134.2 (CH), 135.1 (Cq), 138.5 (Cq), 148.8 (Cq), 152.7 (CH), 157.0 (Cq), 166.0 (Cq); HRMS (EI-MS): C₇H₂³⁵Cl₃N₃, calculated m/z 232.9314. found m/z 232.9323.

### 1.1. Suzuki Coupling in Position 4 for the Compound 2

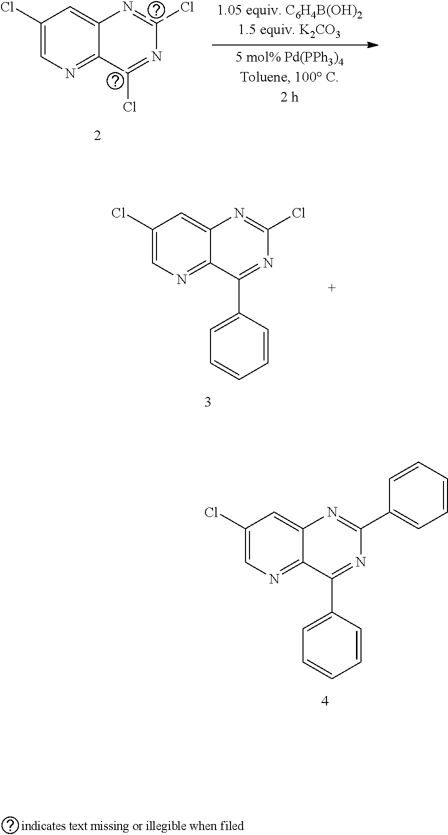 [SMILES: Clc1cnc2c(Cl)nc(Cl)nc2c1 ; Clc1cnc2c(-c3ccccc3)nc(Cl)nc2c1 ; Clc1cnc2c(-c3ccccc3)nc(-c3ccccc3)nc2c1]

### 2,7-Dichloro-4-phenyl-pyrido[3,2-d]pyrimidine (3)

Under an argon atmosphere, in 25 mL flask, 100 mg (0.5 mmol, 1.0 equiv.) of 2 are dissolved in 7 mL of anhydrous toluene, and then 64 mg (0.52 mmol, 1.05 equiv.) of phenylboronic acid, 104 mg (0.75 mmol, 1.5 equiv.) of potassium carbonate and 29 mg (25 μmol, 0.05 equiv.) of tetrakis(triphenylphosphino)palladium(0) are added. The whole is brought to 100° C. for 2 hours. The solvant is evaporated and the residue is taken up with water and extracted with dichloromethane. The organic extracts are dried on MgSO₄ and then concentrated under reduced pressure. The compound 3 is obtained, after purification on a chromatographic silica gel column (petroleum ether/AcOEt, 95/5) as a white solid with a yield of 84%. MP: 140-141° C.; IR (ATR, Diamond, cm⁻¹): ν 1523, 1465, 1279, 1135, 884, 811, 761, 682; ¹H NMR (250 MHz, CDCl₃) δ: 7.53-7.59 (m, 3H, H_{Ph}), 7.83 (dd, 1H, J=4.0 Hz, J=8.7 Hz, H₇), 8.32 (dd, 1H, J=1.5 Hz, J=8.7 Hz, H₈), 8.38 (m, 2H, H_{Ph}), 9.10 (dd, 1H, J=1.5 Hz, J=4.0 Hz, H₆); ¹³C NMR (62.5 MHz, CDCl₃) δ: 128.4 (2CH), 128.6 (CH), 131.7 (CH), 132.0 (2CH), 134.8 (Cq), 136.0 (CH), 137.6 (Cq), 149.6 (Cq), 151.9 (CH), 157.5 (Cq), 169.8 (Cq); HRMS (EI-MS): C₁₃H₈N₃³⁵Cl, calculated m/z 241.0407. found m/z 241.0414.

### 7-Chloro-2,4-diphenyl-pyrido[3,2-d]pyrimidine (4)

The product 4 is obtained as a byproduct of the synthesis of 3 with a yield of 2% as a yellow solid. MP: 120-121° C.; IR (ATR, Diamond, cm⁻¹): ν3027, 1594, 1533, 1439, 1384, 1327, 1159, 1021, 980, 890; ¹H NMR (250 MHz, CDCl₃) δ: 7.54-7.63 (m, 6H, H_{Ph}), 8.38 (d, 1H, J=2.2 Hz, H₈), 8.44-8.48 (m, 2H, H_{Ph}), 8.68-8.72 (m, 2H, H_{Ph}), 8.90 (d, 1H, J=2.2 Hz, H₆); ¹³C NMR (62.5 MHz, CDCl₃) δ: 128.4 (2CH), 128.8 (2CH), 129.1 (2CH), 131.1 (CH), 131.4 (CH), 131.8 (2CH), 135.2 (CH), 135.5 (Cq), 136.0 (Cq), 136.2 (Cq), 137.4 (Cq), 148.5 (Cq), 150.2 (CH), 161.8 (Cq), 166.6 (Cq); HRMS (EI-MS): C₁₉H₁₂³³ClN₃, calculated m/z 318.0798 (M+1). found m/z 318.0798 (M+1).

### 1.2. Arylations in Position 2 of the Compound 3

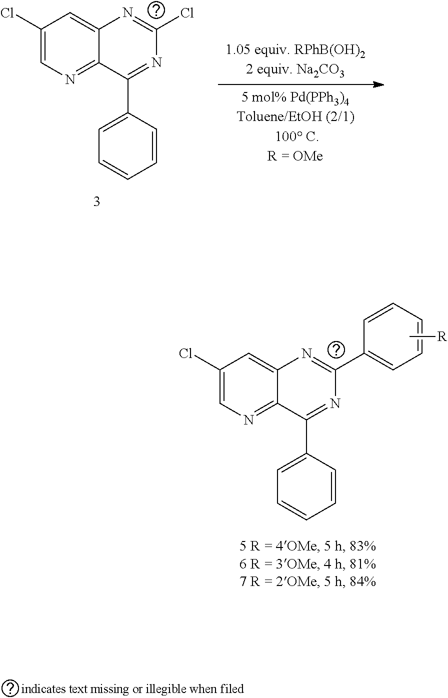

General Procedure A:

In an argon atmosphere, in a 25 mL flask, 1 equiv. of 2-chloro-4-phenylamino-pyrido[3,2-d]pyrimidine 3 is dissolved in anhydrous toluene and ethanol for analysis (2/1), and then 1.2 equiv. of boronic acid, 2.0 equiv. of sodium carbonate and 0.05 equiv. of tetrakis(triphenylphosphino)palladium(0) are added. The mixture is heated to 100° C. for 48 hours. The solvants are evaporated and then the residue is taken up with water and extracted with dichloromethane. The organic extracts are dried on MgSO₄ and then concentrated under reduced pressure.

### 7-Chloro-2-(4-methoxyphenyl)-4-phenyl-pyrido[3,2-d]pyrimidine (5)

The product 5 is synthesized from 3 according to the general procedure A. After purification on a chromatographic silica gel column (AcOEt/petroleum ether, 5/95) it is obtained as a yellow solid with a yield of 83%. MP: 193-194° C.; IR (ATR, Diamond, cm⁻¹) ν 3053, 2167, 1604, 1538, 1443, 1317, 1249, 1164, 1027, 846; ¹H NMR (400 MHz, CDCl₃) δ: 3.90 (s, 3H, OCH₃), 7.03 (d, 2H, J=9.0 Hz, H_{Arom}), 7.57-7.59 (m, 3H, H_{Ph}), 8.32 (d, 1H, J=2.4 Hz, H₈), 8.41-8.44 (m, 2H, H_{Ph}), 8.64 (d, 2H, J=9.0 Hz, H_{Arom}), 8.84 (d, 1H, J=2.4 Hz, H₆); ¹³C NMR (100 MHz, CDCl₃) δ: 55.6 (CH₃), 114.1 (2CH), 128.3 (2CH), 130.1 (Cq), 130.9 (2CH), 131.0 (CH), 131.8 (2CH), 134.9 (CH), 135.4 (Cq), 135.8 (Cq), 136.3 (Cq), 148.6 (Cq), 149.6 (CH), 161.6 (Cq), 162.5 (Cq), 166.5 (Cq); HRMS (EI-MS): C₂₀H₁₄³³ClN₃O, calculated m/z 348.0904 (M+1). found m/z 348.0908 (M+1).

### 7-Chloro-2-(3-methoxyphenyl)-4-phenyl-pyrido[3,2-d]pyrimidine (6)

The product 6 is synthesized from 3 according to the general procedure A. After purification on a silica gel chromatography column (AcOEt/petroleum ether, 2/98) it is obtained as a yellow solid with a yield of 81%. MP: 112-113° C.; IR (ATR, Diamond, cm⁻¹) ν 3053, 2828, 1589, 1536, 1456, 1334, 1248, 1179, 1047, 836; ¹H NMR (250 MHz, CDCl₃) δ: 3.92 (s, 3H, OCH₃), 7.06 (ddd, 1H, J=0.8 Hz, J=2.6 Hz, J=8.2 Hz, H_{Arom}), 7.42 (t, 1H, J=8.0 Hz, H_{Arom}), 7.55-7.58 (m, 3H, H_{Ph}), 8.20 (dd, 1H, J=1.6 Hz, J=2.6 Hz, H_{Arom}), 8.25 (d, 1H, J=8.0 Hz, H_{Arom}), 8.32 (d, 1H, J=2.4 Hz, H₈), 8.40-8.44 (m, 2H, H_{Ph}), 8.85 (d, 1H, J=2.4 Hz, H₆); ¹³C NMR (62.5 MHz, CDCl₃) δ: 55.5 (CH₃), 113.8 (CH), 117.5 (CH), 121.6 (CH), 128.3 (2CH), 129.7 (CH), 131.1 (CH), 131.8 (2CH), 135.1 (CH), 135.4 (Cq), 135.9 (Cq), 136.1 (Cq), 138.7 (Cq), 148.3 (Cq), 150.1 (CH), 160.0 (Cq), 161.5 (Cq), 166.4 (Cq); HRMS (EI-MS): C₂₀H₁₄³⁵ClN₃O, calculated m/z 348.0904 (M+1). found m/z 348.0905 (M+1).

### 7-Chloro-2-(2-methoxyphenyl)-4-phenyl-pyrido[3,2-d]pyrimidine (7)

The product 7 is synthesized from 3 according to the general procedure A. After purification on a silica gel chromatography column (AcOEt/petroleum ether, 05/95) as a yellow solid with a yield of 84%. MP: 116-117° C.; IR (ATR, Diamond, cm⁻¹) ν 3068, 2267, 1584, 1538, 1445, 1379, 1292, 1113, 1077, 887; ¹H NMR (250 MHz, CDCl₃) δ: 3.93 (s, 3H, OCH₃), 7.07-7.15 (m, 2H, H_{Arom}), 7.44-7.51 (m, 1H, H_{Arom}), 7.54-7.58 (m, 3H, H_{Ph}), 7.93 (dd, 1H, J=1.7, 7.5 Hz, H_{Arom}), 8.36-8.40 (m, 2H, H_{Ph}), 8.43 (d, 1H, J=2.4 Hz, H₈), 8.95 (d, 1H, J=2.4 Hz, H₆); ¹³C NMR (62.5 MHz, CDCl₃) δ: 56.2 (CH₃), 112.4 (CH), 120.9 (CH), 128.3 (2CH), 128.4 (Cq), 131.0 (CH), 131.5 (CH), 131.8 (2CH), 132.2 (CH), 135.2 (CH), 135.4 (Cq), 135.5 (Cq), 136.1 (Cq), 148.1 (Cq), 150.5 (CH), 158.2 (Cq), 163.5 (Cq), 166.7 (Cq); HRMS (EI-MS): C₂₀H₁₄³⁵ClN₃O, calculated m/z 348.0904 (M+1). found m/z 348.0900 (M+1).

### 1.3. Obtaining Compounds with tris(het)aryls in Positions 2, 4 and 7

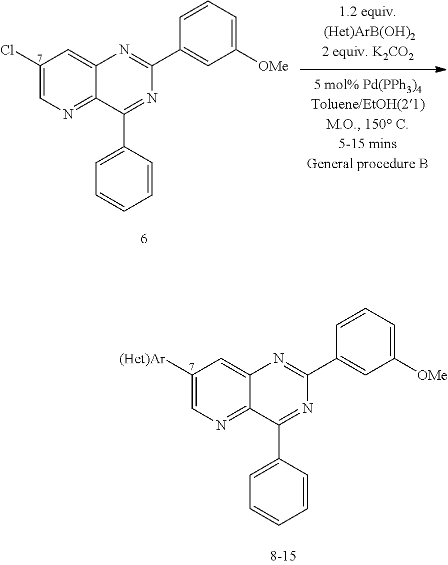

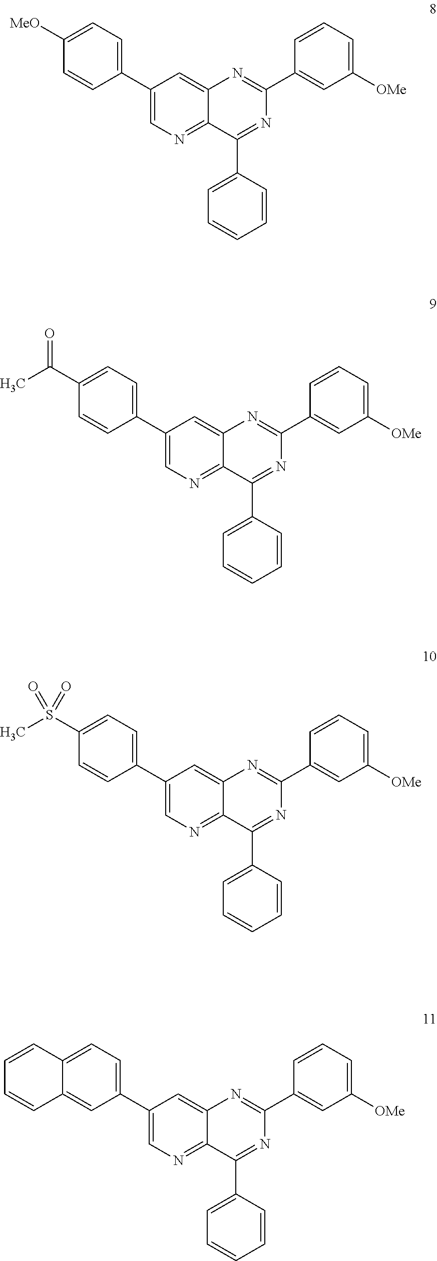 [SMILES: COc1ccc(-c2cnc3c(-c4ccccc4)nc(-c4cccc(OC)c4)nc3c2)cc1 ; COc1cccc(-c2nc(-c3ccccc3)c3ncc(-c4ccc(C(C)=O)cc4)cc3n2)c1 ; COc1cccc(-c2nc(-c3ccccc3)c3ncc(-c4ccc(S(C)(=O)=O)cc4)cc3n2)c1 ; COc1cccc(-c2nc(-c3ccccc3)c3ncc(-c4ccc5ccccc5c4)cc3n2)c1]

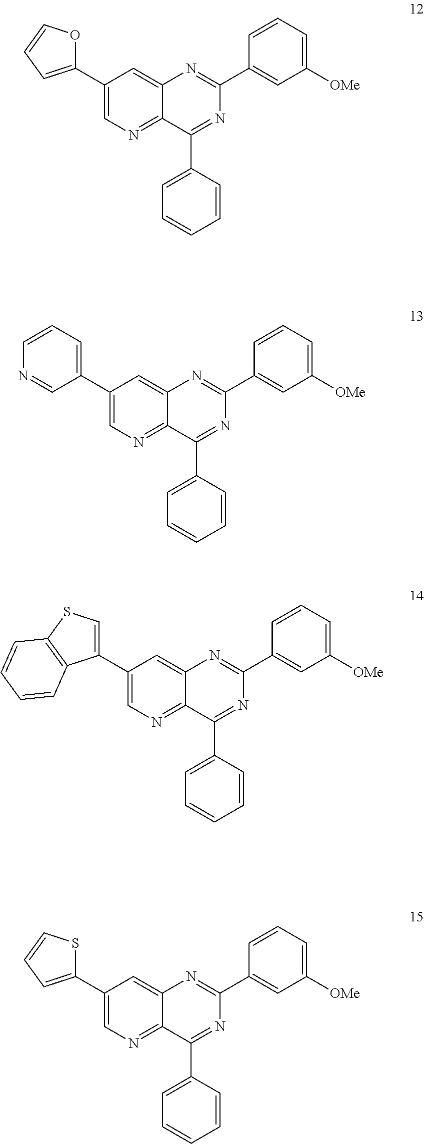 [SMILES: COc1cccc(-c2nc(-c3ccccc3)c3ncc(-c4ccco4)cc3n2)c1 ; COc1cccc(-c2nc(-c3ccccc3)c3ncc(-c4cccnc4)cc3n2)c1 ; COc1cccc(-c2nc(-c3ccccc3)c3ncc(-c4csc5ccccc45)cc3n2)c1 ; COc1cccc(-c2nc(-c3ccccc3)c3ncc(-c4cccs4)cc3n2)c1]

General Procedure B.

The products are synthesized by using the general procedure A at 150° C. with microwave irradiation for 5 minutes from 6 by replacing Na₂CO₃ with K₂CO₃. The solvants are evaporated and then the residue is taken up with water and extracted with dichloromethane. The organic extracts are dried on MgSO₄ and then concentrated under reduced pressure.

### 2-(3-Methoxyphenyl)-7-(4-methoxyphenyl)-4-phenyl-pyrido[3,2-d]-pyrimidine (8)

The product 8 is synthesized from 6 according to the general procedure B after purification on a chromatographic silica gel column (CH₂Cl₂/petroleum ether, 15/55) as a yellow solid with a yield of 94%. MP: 147-148° C.; IR (ATR, Diamond, cm⁻¹) ν 3002, 2828, 1604, 1543, 1451, 1333, 1230, 1169, 1021, 836; ¹H NMR (250 MHz, CDCl₃) δ: 3.75 (s, 3H, OCH₃), 3.84 (s, 3H, OCH₃), 6.92-6.98 (m, 3H, H_{Arom}), 7.34 (t, 1H, J=8.0 Hz, H_{Arom}), 7.46-7.50 (m, 3H, H_{Ph}), 7.59 (d, 2H, J=8.8 Hz, H_{Arom}), 8.16-8.18 (m, 1H, H_{Arom}), 8.21 (d, 1H, J=7.8 Hz, H_{Arom}), 8.32 (d, 1H, J=2.3 Hz, H₈), 8.38-8.42 (m, 2H, H_{Ph}), 9.11 (d, 1H, J=2.3 Hz, H₆); ¹³C NMR (62.5 MHz, CDCl₃) δ: 55.50 (CH₃), 55.53 (CH₃), 113.7 (CH), 114.9 (2CH), 117.1 (CH), 121.5 (CH), 128.2 (2CH), 128.6 (Cq), 128.8 (2CH), 129.7 (CH), 130.7 (CH), 131.8 (2CH), 132.2 (CH), 136.4 (Cq), 136.6 (Cq), 139.3 (Cq), 139.8 (Cq), 148.3 (Cq), 150.2 (CH), 160.0 (Cq), 160.7 (Cq), 160.9 (Cq), 165.9 (Cq); HRMS (EI-MS): C₂₇H₂₁N₃O₂, calculated m/z 420.1712 (M+1). found m/z 420.1717 (M+1).

### 7-(4-Acetylphenyl)-2-(3-methoxyphenyl)-4-phenyl-pyrido[3,2-d]pyrimidine (9)

The product 9 is synthesized from 6 according to the general procedure B after purification on a chromatographic silica gel column (CH₂Cl₂/petroleum ether, 15/55) as a yellow solid with a yield of 97%. MP: 133-134° C.; IR (ATR, Diamond, cm⁻¹) ν 2935, 1681, 1604, 1536, 1454, 1340, 1265, 1047, 908, 830; ¹H NMR (250 MHz, CDCl₃) δ: 2.61 (s, 3H, CH₃), 3.89 (s, 3H, OCH₃), 7.03 (ddd, 1H, J=0.9, 2.7, 8.2 Hz, H_{Arom}), 7.40 (t, 1H, J=8.0 Hz, H_{Arom}), 7.52-7.55 (m, 3H, H_{Ph}), 7.82 (d, 2H, J=8.5 Hz, H_{Arom}), 8.08 (d, 2H, J=8.5 Hz, H_{Arom}), 8.21-8.23 (m, 1H, H_{Arom}), 8.27 (d, 1H, J=7.8 Hz, H_{Arom}), 8.43-8.47 (m, 2H, H_{Ph}), 8.52 (d, 1H, J=2.3 Hz, H₈), 9.21 (d, 1H, J=2.3 Hz, H₆); ¹³C NMR (62.5 MHz, CDCl₃) δ: 26.7 (CH₃), 55.4 (CH₃), 113.7 (CH), 117.1 (CH), 121.4 (CH), 127.7 (2CH), 128.2 (2CH), 129.3 (2CH), 129.6 (CH), 130.9 (CH), 131.8 (2CH), 133.9 (CH), 136.3 (Cq), 137.0 (Cq), 137.2 (Cq), 138.7 (Cq), 138.9 (Cq), 140.5 (Cq), 147.8 (Cq), 149.6 (CH), 159.9 (Cq), 160.9 (Cq), 165.8 (Cq), 197.3 (Cq); HRMS (EI-MS): C₂₈H₂₁N₃O₂, calculated m/z 432.1712 (M+1). found m/z 432.1709 (M+1).

### 7-(4-Methanesulfonyl)-2-(3-methoxyphenyl)-4-phenyl-pyrido[3,2-d]pyrimidine (10)

The product 10 is synthesized from 6 according to the general procedure B after purification on a chromatographic silica gel column

(AcOEt/petroleum ether, 20/80) as a yellow solid with a yield of 97%. MP: 215-216° C.; IR (ATR, Diamond, cm⁻¹) ν 2920, 1594, 1535, 1460, 1301, 1225, 1148, 1034, 956, 852; ¹H NMR (250 MHz, CDCl₃) δ: 3.33 (s, 3H, CH₃), 3.87 (s, 3H, OCH₃), 7.14 (dd, 1H, J=2.5, 8.1 Hz, H_{Arom}), 7.49 (t, 1H, J=8.0 Hz, H_{Arom}), 7.60-7.64 (m, 3H, H_{Ph}), 8.09-8.13 (m, 3H, H_{Arom}), 8.21 (d, 1H, J=7.8 Hz, H_{Arom}), 8.28 (d, 2H, J=8.4 Hz, H_{Arom}), 8.43-8.47 (m, 2H, H_{Ph}), 8.78 (d, 1H, J=2.2 Hz, H₈), 9.46 (d, 1H, J=2.2 Hz, H₆); ¹³C NMR (62.5 MHz, CDCl₃) δ: 43.4 (CH₃), 55.2 (CH₃), 113.3 (CH), 116.9 (CH), 120.8 (CH), 127.8 (2CH), 128.1 (2CH), 128.8 (2CH), 129.9 (CH), 130.8 (CH), 131.6 (2CH), 134.0 (CH), 135.9 (Cq), 136.6 (Cq), 138.0 (Cq), 138.4 (Cq), 140.4 (Cq), 141.3 (Cq), 147.4 (Cq), 150.6 (CH), 159.7 (Cq), 159.8 (Cq), 165.5 (Cq); HRMS (EI-MS): C₂₇H₂₁N₃O₃S, calculated m/z 468.1382 (M+1). found m/z 468.1384 (M+1).

### 2-(3-Methoxyphenyl)-7-(naphthyl)-4-phenyl-pyrido[3,2-d]pyrimidine (11)

The product 11 is synthesized from 6 according to the general procedure B after purification on a chromatographic silica gel column (CH₂Cl₂/petroleum ether, 2/8) as a yellow solid with a yield of 98%. MP: 166-167° C.; IR (ATR, Diamond, cm⁻¹) ν 3063, 2833, 1584, 1532, 1445, 1338, 1276, 1220, 1046, 826; ¹H NMR (250 MHz, CDCl₃) δ: 3.82 (s, 3H, OCH₃), 6.95 (dd, 1H, J=2.6, 8.1 Hz, H_{Arom}), 7.33 (t, 1H, J=8.0 Hz, H_{Arom}), 7.40-7.43 (m, 2H, H_{Naph}), 7.47-7.50 (m, 3H, H_{Ph}), 7.68-7.86 (m, 4H, H_{Naph}), 8.06 (s, 1H, H_{Naph}), 8.16-8.17 (m, 1H, H_{Arom}), 8.22 (d, 1H, J=7.8 Hz, H_{Arom}), 8.40-8.45 (m, 3H, H_{Ph} and H₈), 9.23 (d, 1H, J=2.3 Hz, H₆); ¹³C NMR (62.5 MHz, CDCl₃) δ: 55.5 (CH₃), 113.7 (CH), 117.2 (CH), 121.6 (CH), 124.8 (CH), 126.9 (CH), 127.1 (CH), 127.2 (CH), 127.8 (CH), 128.2 (2CH), 128.6 (CH), 129.4 (CH), 129.7 (CH), 130.8 (CH), 131.9 (2CH), 133.4 (Cq), 133.5 (CH), 133.6 (Cq), 136.6 (Cq), 136.8 (Cq), 139.2 (Cq), 140.1 (Cq), 148.2 (Cq), 150.4 (CH), 160.0 (2Cq), 160.9 (Cq), 166.0 (Cq); HRMS (EI-MS): C₃₀H₂₁N₃O, calculated m/z 440.1763 (M+1). found m/z 440.1766 (M+1).

### 7-(2-Furyl)-2-(3-methoxyphenyl)-4-phenyl-pyrido[3,2-d]pyrimidine (12)

The product 8 is synthesized from 6 according to the general procedure B after purification on a chromatographic silica gel column (CH₂Cl₂/petroleum ether, 2/8) as a yellow solid with a yield of 97%. MP: 131-132° C.; IR (ATR, Diamond, cm⁻¹) ν 2956, 1599, 1531, 1451, 1340, 1229, 1176, 1034, 897, 826; ¹H NMR (250 MHz, CDCl₃) δ: 3.85 (s, 3H, OCH₃), 6.48 (dd, 1H, J=1.8, 3.4 Hz, H_{Hét}), 6.90 (d, 1H, J=3.4 Hz, H_{Hét}), 6.97 (dd, 1H, J=1.9, 8.1 Hz, H_{Arom}), 7.34 (t, 1H, J=8.0 Hz, H_{Arom}), 7.47-7.52 (m, 4H, H_{Ph} and H_{Hét}), 8.15-8.17 (m, 1H, H_{Arom}), 8.21 (d, 1H, J=7.8 Hz, H_{Arom}), 8.35-8.40 (m, 3H, H_{Ph} and H₈), 9.16 (d, 1H, J=2.2 Hz, H₆); ¹³C NMR (62.5 MHz, CDCl₃) δ: 55.6 (CH₃), 109.7 (CH), 112.5 (CH), 113.7 (CH), 117.2 (CH), 121.6 (CH), 128.2 (2CH), 128.9 (CH), 129.7 (CH), 130.1 (Cq), 130.8 (CH), 131.8 (2CH), 136.4 (Cq), 136.5 (Cq), 139.2 (Cq), 144.7 (CH), 147.4 (CH), 148.3 (Cq), 150.2 (Cq), 160.0 (Cq), 161.1 (Cq), 165.9 (Cq); HRMS (EI-MS): C₂₄H₁₇N₃O₂, calculated m/z 380.1399 (M+1). found m/z 380.1416 (M+1).

### 2-(3-Methoxyphenyl)-4-phenyl-7-(3-pyridyl)-pyrido[3,2-d]pyrimidine (13)

The product 13 is synthesized from 6 according to the general procedure B after purification on a chromatographic silica gel column (CH₂Cl₂/MeOH, 99.5/0.5) as a yellow solid with a yield of 88%. MP: 134-135° C.; IR (ATR, Diamond, cm⁻¹) ν 3048, 2162, 1589, 1533, 1453, 1394, 1341, 1230, 1026, 841; ¹H NMR (250 MHz, CDCl₃) δ: 3.92 (s, 3H, OCH₃), 7.05 (ddd, 1H, J=0.9 Hz, J=2.6 Hz, J=8.2 Hz, H_{Arom}), 7.39-7.48 (m, 2H, H_{Arom} and H_{Pyr}), 7.57-7.60 (m, 3H, H_{Ph}), 8.02 (d, 1H, J=7.9 Hz, H_{Pyr}), 8.22-8.24 (m, 1H, H_{Arom}), 8.28 (d, 1H, J=7.8 Hz, H_{Arom}), 8.47-8.52 (m, 3H, H_{Ph} and H₈), 8.75 (sl, 1H, H_{Pyr}), 9.04 (sl, 1H, H_{Pyr}), 9.18 (d, 1H, J=2.3 Hz, H₆); ¹³C NMR (62.5 MHz, CDCl₃) δ: 55.5 (CH₃), 113.7 (CH), 117.3 (CH), 121.5 (CH), 128.3 (2CH), 129.7 (CH), 130.9 (CH), 131.8 (2CH), 134.0 (CH), 134.8 (CH), 136.3 (Cq), 137.1 (Cq), 137.2 (Cq), 138.9 (Cq), 147.9 (Cq), 148.5 (CH), 149.5 (2CH), 150.3 (CH), 160.0 (2Cq), 161.1 (Cq), 166.2 (Cq); HRMS (EI-MS): C₂₅H₁₈N₄O, calculated m/z 391.1559 (M+1). found m/z 391.1565 (M+1).

### 7-(3-Benzothienyl)-2-(3-methoxyphenyl)-4-phenyl-pyrido[3,2-d]pyrimidine (14)

The product 14 is isolated after purification on a chromatographic silica gel column (CH₂Cl₂/petroleum ether, 3/7) as a yellow solid with a yield of 96%. MP: 112-113° C.; IR (ATR, Diamond, cm⁻¹) ν 2920, 1599, 1538, 1505, 1448, 1334, 1226, 1039, 908, 831; ¹H NMR (250 MHz, CDCl₃) δ: 3.96 (s, 3H, OCH₃), 7.09 (ddd, 1H, J=0.9 Hz, J=2.6 Hz, J=8.2 Hz, H_{Arom}), 7.43-7.51 (m, 3H, H_{Arom} and H_{Hét}), 7.61-7.64 (m, 3H, H_{Ph}), 7.70 (s, 1H, H_{Hét}), 7.95-7.99 (m, 1H, H_{Hét}), 8.04-8.08 (m, 1H, H_{Hét}), 8.30-8.32 (m, 1H, H_{Arom}), 8.36 (d, 1H, J=7.8 Hz, H_{Arom}), 8.54-8.58 (m, 3H, H_{Ph} and H₈), 9.24 (d, 1H, J=2.2 Hz, H₆); ¹³C NMR (62.5 MHz, CDCl₃) δ: 55.5 (CH₃), 113.7 (CH), 117.2 (CH), 121.6 (CH), 122.4 (CH), 123.3 (CH), 125.2 (2CH), 126.8 (CH), 128.3 (2CH), 129.7 (CH), 130.9 (CH), 131.9 (2CH), 133.2 (Cq), 134.8 (CH), 135.7 (Cq), 136.5 (Cq), 136.9 (Cq), 137.0 (Cq), 139.2 (Cq), 140.9 (Cq), 148.2 (Cq), 151.2 (CH), 160.0 (Cq), 161.0 (Cq), 166.2 (Cq); HRMS (EI-MS): C₂₈H₁₉N₃OS, calculated m/z 446.1327 (M+1). found m/z 446.1329 (M+1).

### 2-(3-Methoxyphenyl)-4-phenyl-7-(2-thienyl)-pyrido[3,2-d]pyrimidine (15)

The product 15 is isolated after purification on a chromatographic silica gel column (CH₂Cl₂/petroleum ether, 2/8) as a yellow solid with a yield of 67%. MP: 140-141° C.; IR (ATR, Diamond, cm⁻¹) ν 3073, 2203, 1599, 1537, 1452, 1335, 1274, 1218, 1042, 894; ¹H NMR (250 MHz, CDCl₃) δ: 3.85 (s, 3H, OCH₃), 6.98 (dd, 1H, J=2.0 Hz, J=8.2 Hz, H_{Arom}), 7.09 (dd, 1H, J=3.7 Hz, J=5.0 Hz, H_{Hét}), 7.32-7.39 (m, 2H, H_{Arom} and H_{Hét}), 7.46-7.52 (m, 4H, H_{Ph} and H_{Hét}), 8.15-8.17 (m, 1H, H_{Arom}), 8.21 (d, 1H, J=7.8 Hz, H_{Arom}), 8.35 (d, 1H, J=2.3 Hz, H₈), 8.37-8.41 (m, 2H, H_{Ph}), 9.17 (d, 1H, J=2.3 Hz, H₆); ¹³C NMR (62.5 MHz, CDCl₃) δ: 55.6 (CH₃), 113.7 (CH), 117.3 (CH), 121.6 (CH), 126.3 (CH), 128.1 (CH), 128.3 (2CH), 128.9 (CH), 129.7 (CH), 130.8 (CH), 130.9 (CH), 131.8 (2CH), 133.9 (Cq), 136.5 (Cq), 136.7 (Cq), 139.2 (Cq), 139.3 (Cq), 148.3 (Cq), 148.8 (CH), 160.1 (Cq), 161.2 (Cq), 165.9 (Cq); HRMS (EI-MS): C₂₄H₁₇N₃OS, calculated m/z 396.1171 (M+1). found m/z 396.1181 (M+1).

### 1.4. Synthesis of 2,7-dichloropyrido[3,2-d]pyrimidine (16)

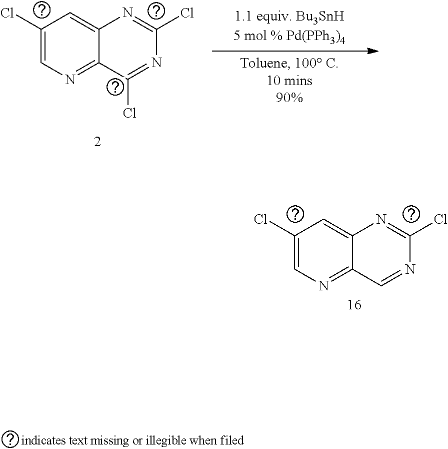 [SMILES: Clc1cnc2c(Cl)nc(Cl)nc2c1 ; Clc1cnc2cnc(Cl)nc2c1]

### 2,7-Dichloropyrido[3,2-d]pyrimidine (16)

Under an argon atmosphere, 1.165 g, (5.0 mmol, 1.0 equiv.) of 2 are dissolved in 60 mL of anhydrous toluene and then 1.6 g (5.50 mmol, 1.1 equiv.) of tributyltin hydride are added dropwise followed by 288 mg (0.25 mmol, 0.05 equiv.) of tetrakis(triphenylphosphino)palladium(0). The whole is brought to 100° C. for 1 hour. Next, the toluene is evaporated and the obtained residue is solubilized in dichloromethane and is hydrolyzed with a saturated potassium fluoride solution. The whole is stirred intensively for 30 minutes and then filtered on celite while rinsing with dichloromethane. The aqueous phase is extracted with dichloromethane. The organic phase is dried on MgSO₄, filtered, and then concentrated under reduced pressure. The thereby obtained residue is chromatographed on silica gel (AcOEt/petroleum ether 5/95) in order to obtain a yellow solid with a yield of 90%. MP: 177-178° C.; IR (ATR, Diamond, cm⁻¹) ν 3043, 2167, 1594, 1538, 1433, 1353, 1215, 1117, 1072, 910; ¹H NMR (250 MHz, DMSO-d₆) δ: 8.68 (d, 1H, J=2.3 Hz, H₈), 9.17 (d, 1H, J=2.3 Hz, H₆), 9.68 (s, 1H, H₄); ¹³C NMR (62.5 MHz, DMSO-d₆) δ: 133.7 (CH), 136.5 (Cq), 137.1 (Cq), 148.0 (Cq), 152.7 (CH), 157.5 (Cq), 164.9 (CH); HRMS (EI-MS): C₇H₃³⁵Cl₂N₃, calculated m/z 198.9704. found m/z 198.9715.

### 1.5. Obtaining the 2-aryl-7-chloro compounds with bis(het)aryl in positions C-2 and C-7

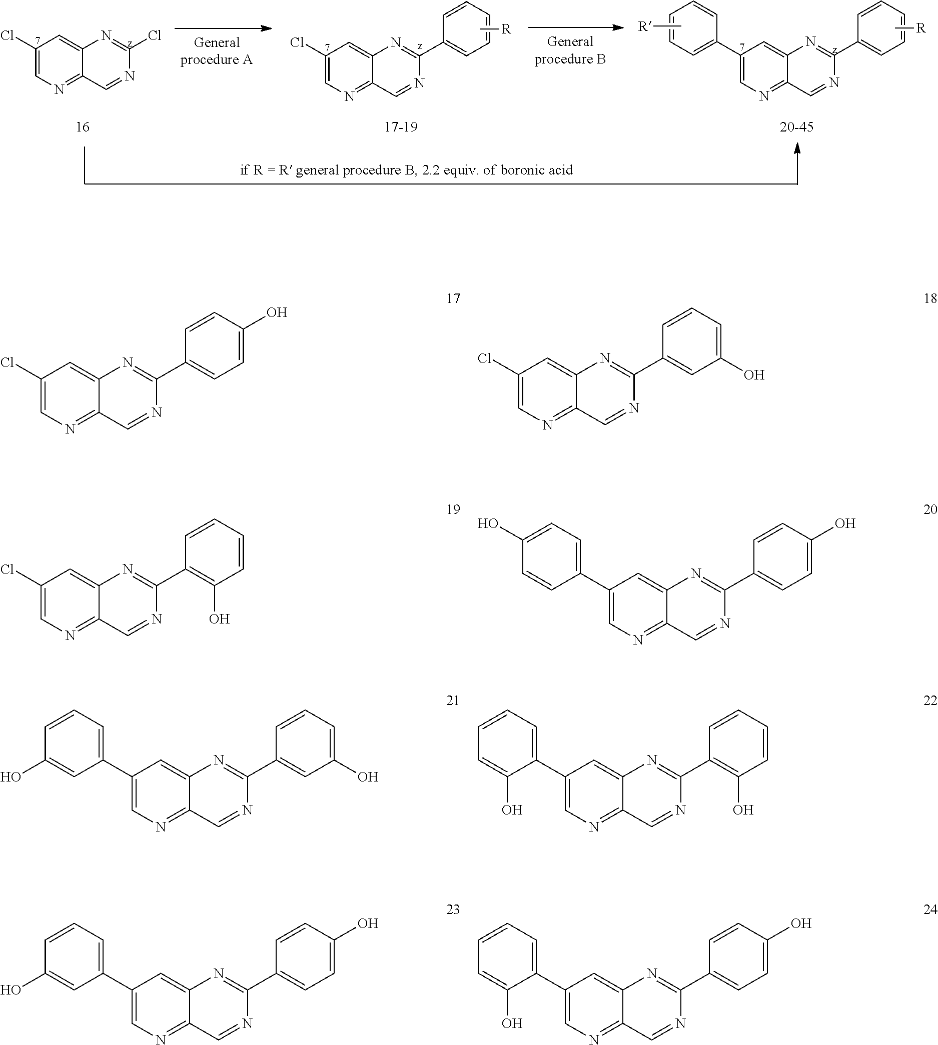

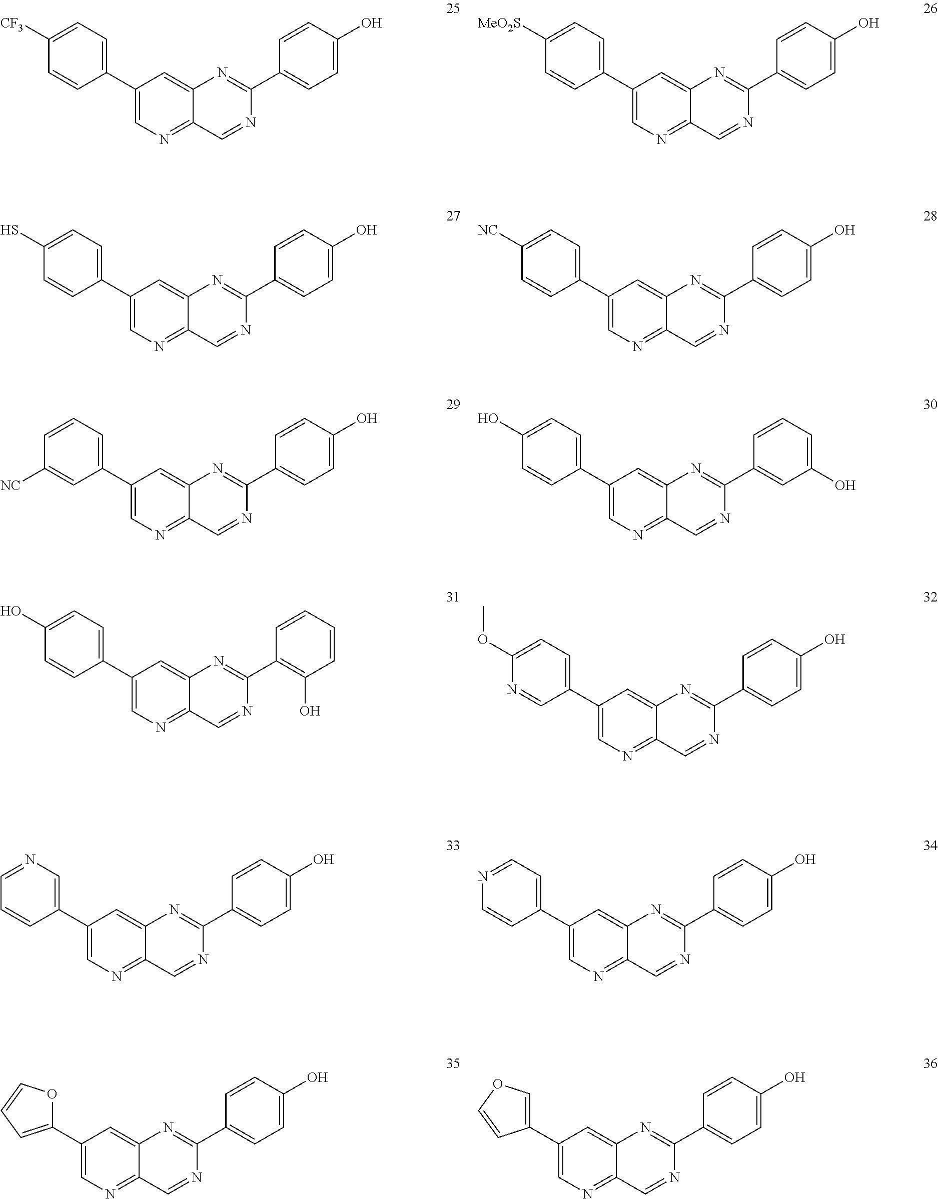

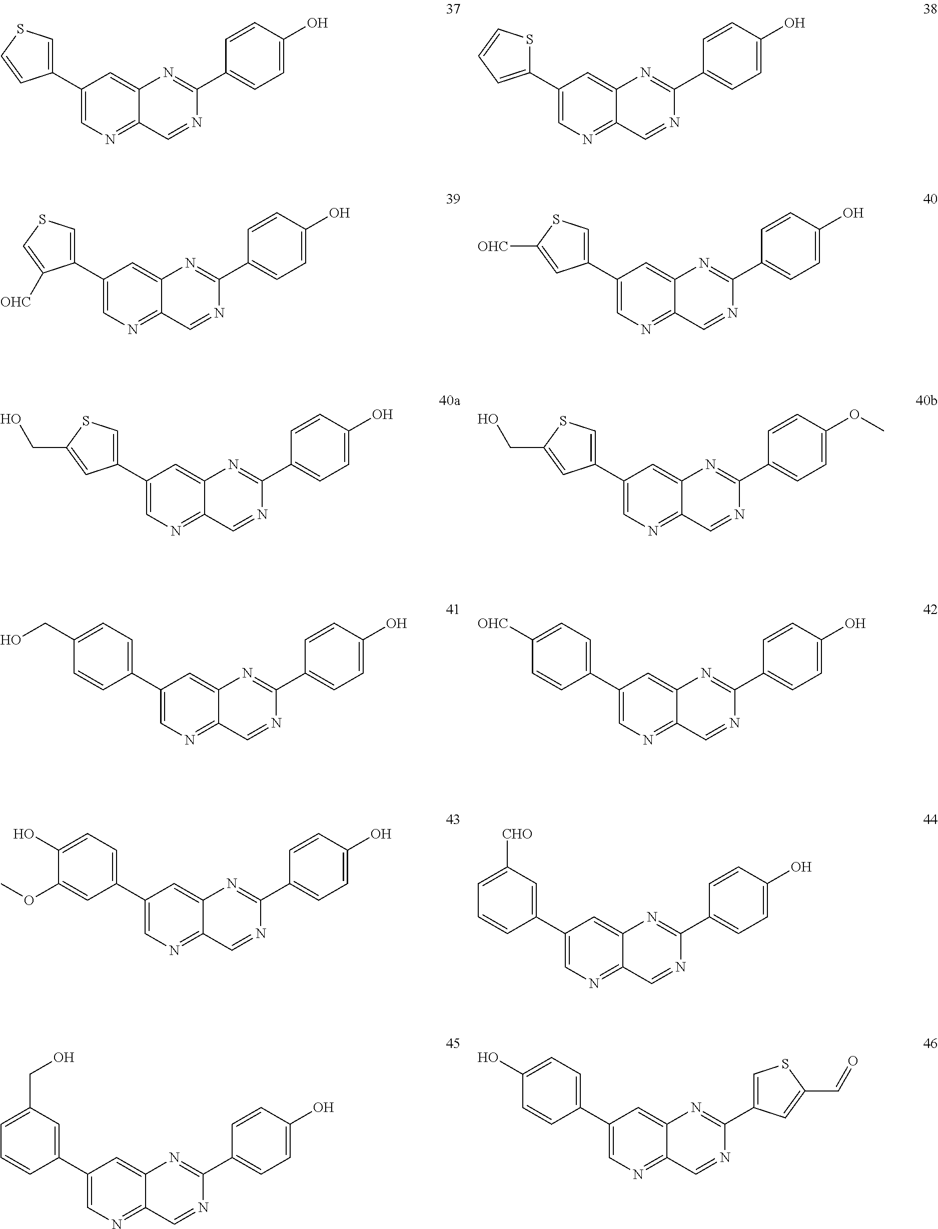

### 7-Chloro-2-(4-hydroxyphenyl)-pyrido[3,2-d]pyrimidine (17)

The product 17 is synthesized from 16 by following the general procedure A and then isolated after purification on a chromatographic silica gel column (CH₂Cl₂/MeOH, 99/1) as a yellow solid with a yield of 63%. MP: 231-232° C.; pmIR (ATR, Diamond, cm⁻¹) ν 3058, 2044, 1587, 1527, 1445, 1353, 1220, 1160, 1077, 893; ¹H NMR (400 MHz, DMSO-d₆) δ: 6.93 (d, 2H, J=8.8 Hz, H_{Arom}), 8.38 (d, 2H, J=8.8 Hz, H_{Arom}), 8.53 (d, 1H, J=2.4 Hz, H₈), 8.99 (d, 1H, J=2.4 Hz, H₆), 9.63 (s, 1H, H₄), 10.15 (s, 1H, OH); ¹³C NMR (100 MHz, DMSO-d₆) δ: 115.6 (2CH), 127.3 (Cq), 130.4 (2CH), 134.0 (CH), 135.2 (Cq), 136.6 (Cq), 146.4 (Cq), 150.5 (CH), 160.7 (Cq), 161.2 (Cq), 161.4 (CH); HRMS (EI-MS): C₁₃H₈³⁵ClN₃O, calculated m/z 258.0434 (M+1). found m/z 258.0439 (M+1).

### 7-Chloro-2-(3-hydroxyphenyl)-pyrido[3,2-d]pyrimidine (18)

The product 18 is synthesized from 16 by following the general procedure A and then isolated after purification on a chromatographic silica gel column (CH₂Cl₂/MeOH, 99.5/0.5) as a yellow solid with a yield of 70%. MP: 252-253° C.; IR (ATR, Diamond, cm⁻¹) ν 3217, 2362, 1599, 1543, 1440, 1379, 1317, 1261, 1041, 882; ¹H NMR (400 MHz, DMSO-d₆) δ: 6.95-6.97 (m, 1H, H_{Arom}), 7.33-7.37 (m, 1H, H_{Arom}), 7.94-7.96 (m, 2H, H_{Arom}), 8.58 (d, 1H, J=1.5 Hz, H₈), 9.04 (d, 1H, J=1.5 Hz, H₆), 9.69 (s, 1H, H₄), 9.72 (s, 1H, OH); ¹³C NMR (100 MHz, DMSO-d₆) δ: 115.1 (CH), 118.6 (CH), 119.4 (CH), 129.8 (CH), 134.4 (CH), 135.4 (Cq), 137.0 (Cq), 137.7 (Cq), 146.3 (Cq), 151.4 (CH), 157.8 (Cq), 161.1 (Cq), 161.6 (CH); HRMS (EI-MS): C₁₃H₈³⁵ClN₃O, calculated m/z 258.0434 (M+1). found m/z 258.0447 (M+1).

### 7-Chloro-2-(2-hydroxyphenyl)-pyrido[3,2-d]pyrimidine (19)

The product 19 is synthesized by following the general procedure A and then isolated after purification on a chromatographic silica gel column (CH₂Cl₂) as a yellow solid with a yield of 66%. MP: 211-212° C.; IR (ATR, Diamond, cm⁻¹) ν 3043, 2259, 1593, 1452, 1362, 1246, 1164, 1080, 952, 827; ¹H NMR (250 MHz, CDCl₃) δ: 6.99-7.09 (m, 2H, H_{Arom}), 7.45 (dt, 1H, J=1.5 Hz, J=8.4 Hz, H_{Arom}), 8.30 (d, 1H, J=2.1 Hz, H₈), 8.64 (dd, 1H, J=1.5 Hz, J=8.0 Hz, H_{Arom}), 8.92 (d, 1H, J=2.1 Hz, H₆), 9.66 (s, 1H, H₄), 12.98 (s, 1H, OH); ¹³C NMR (100 MHz, CDCl₃) δ: 118.3 (CH), 118.6 (Cq), 119.6 (CH), 130.4 (CH), 133.3 (CH), 134.4 (CH), 136.7 (Cq), 137.2 (Cq), 145.2 (Cq), 151.6 (CH), 161.1 (Cq), 161.5 (CH), 163.3 (Cq); HRMS (EI-MS): C₁₃H₈³⁵ClN₃O, calculated m/z 258.0434 (M+1). found m/z 258.0427 (M+1).

### 2,7-Di-(4-hydroxyphenyl)-pyrido[3,2-d]pyrimidine (20)

The product 20 is synthesized from 16 by following the general procedure B with 2.2 equiv. of 4-hydroxyphenyl boronic acid for 15 mins and then isolated after purification on a chromatographic silica gel column (CH₂Cl₂) as a red solid with a yield of 73%. MP: 307-308° C.; IR (ATR, Diamond, cm⁻¹) ν 3039, 2085, 1575, 1513, 1453, 1337, 1220, 1167, 1015, 841; ¹H NMR (400 MHz, DMSO-d₆) δ: 6.93-6.98 (m, 4H, H_{Arom}), 7.87 (d, 2H, J=13.9 Hz, H_{Arom}), 8.41-8.46 (m, 3H, H_{Arom} and H₈), 9.34 (d, 1H, J=3.4 Hz, H₆), 9.60 (s, 1H, H₄), 9.98 (sl, 1H, OH), 10.10 (sl, 1H, OH); ¹³C NMR (100 MHz, DMSO-d₆) δ: 115.6 (2CH), 116.3 (2CH), 126.1 (Cq), 127.8 (Cq), 129.1 (2CH), 129.9 (CH), 130.3 (2CH), 136.8 (Cq), 140.4 (Cq), 146.7 (Cq), 150.6 (CH), 159.0 (Cq), 160.6 (Cq), 160.8 (Cq), 161.0 (CH); HRMS (EI-MS): C₁₉H₃O₂, calculated m/z 316.1086 (M+1). found m/z 316.1085 (M+1).

### 2,7-Di-(3-hydroxyphenyl)-pyrido[3,2-d]pyrimidine (21)

The product 21 is synthesized from 16 by following the general procedure B with 2.2 equiv. of 3-hydroxyphenyl boronic acid for 15 mins and then isolated after purification on a chromatographic silica gel column (CH₂Cl₂) as a yellow solid with a yield of 79%. MP: 264-265° C.; IR (ATR, Diamond, cm⁻¹) ν 3345, 2920, 2280, 1580, 1553, 1455, 1394, 1246, 1179, 1026, 872; ¹H NMR (400 MHz, DMSO-d₆) δ: 7.00-7.08 (m, 4H, H_{Arom}), 7.33-7.37 (m, 1H, H_{Arom}), 7.45-7.49 (m, 1H, H_{Arom}), 7.61 (dd, 1H, J=1.2 Hz, J=7.6 Hz, H_{Arom}), 7.59 (dd, 1H, J=1.5 Hz, J=8.1 Hz, H_{Arom}), 8.65 (d, 1H, J=1.6 Hz, H₈), 9.35 (d, 1H, J=1.6 Hz, H₆), 9.79 (s, 1H, H₄), 10.21 (s, 1H, OH), 13.34 (s, 1H, OH); ¹³C NMR (100 MHz, DMSO-d₆) δ: 116.4 (CH), 117.8 (CH), 118.5 (Cq), 119.2 (CH), 120.0 (CH), 122.7 (Cq), 129.6 (CH), 131.0 (CH), 131.1 (CH), 132.9 (CH), 133.7 (CH), 136.4 (Cq), 140.5 (Cq), 144.6 (Cq), 153.8 (CH), 155.0 (Cq), 160.2 (Cq), 161.2 (Cq), 161.5 (CH); HRMS (EI-MS): C₁₉H₁₃N₃O₂, calculated m/z 316.1086 (M+1). found m/z 316.1096 (M+1).

### 2,7-Di-(2-hydroxyphenyl)-pyrido[3,2-d]pyrimidine (22)

The product 22 is synthesized from 16 by following the general procedure B with 2.2 equiv. of 2-hydroxyphenyl boronic acid for 15 mins and then isolated after purification on a chromatographic silica gel column (CH₂Cl₂) as a yellow solid with a yield of 62%. MP: 239-240° C.; IR (ATR, Diamond, cm⁻¹) ν 3043, 2925, 2029, 1589, 1538, 1445, 1369, 1253, 1026, 954, 826. ¹H NMR (400 MHz, DMSO-d₆) δ: 6.93-6.98 (m, 2H, H_{Arom}), 7.32 (s, 1H, H_{Arom}), 7.36-7.40 (m, 3H, H_{Arom}), 8.02-8.04 (m, 2H, H_{Arom}), 8.54 (d, 1H, J=1.5 Hz, H₈), 9.36 (d, 1H, J=1.5 Hz, H₆), 9.71 (s, 1H, OH), 9.72 (s, 1H, H₄), 9.79 (s, 1H, OH); ¹³C NMR (100 MHz, DMSO-d₆) δ: 114.5 (CH), 115.1 (CH), 116.4 (CH), 118.3 (CH), 118.6 (CH), 119.3 (CH), 129.8 (CH), 130.5 (CH), 131.9 (CH), 137.0 (Cq), 137.8 (Cq), 138.2 (Cq), 140.7 (Cq), 146.4 (Cq), 151.6 (CH), 157.8 (Cq), 158.2 (Cq), 160.7 (Cq), 161.4 (CH); HRMS (EI-MS): C₁₉H₁₃N₃O₂, calculated m/z 316.1086 (M+1). found m/z 316.1098 (M+1).

### 2-(4-Hydroxyphenyl)-7-(3-hydroxyphenyl)-pyrido[3,2-d]pyrimidine (23)

The product 23 is synthesized from 17 by following the general procedure B for 15 mins and then isolated after purification on a chromatographic silica gel column (MeOH/CH₂Cl₂, 02/98) as a yellow solid with a yield of 82%. MP: 256-257° C.; IR (ATR, Diamond, cm⁻¹) ν 3085, 1582, 1550, 1451, 1398, 1373, 1246, 1162, 944, 836; ¹H NMR (400 MHz, DMSO-d₆) δ: 6.93-6.98 (m, 3H, H_{Arom}), 7.32 (s, 1H, H_{Arom}), 7.40 (d, 2H, J=7.9 Hz, H_{Arom}), 8.46 (d, 2H, J=13.9 Hz, H_{Arom}), 8.49 (d, 1H, J=3.0 Hz, H₈), 9.31 (d, 1H, J=3.0 Hz, H₆), 9.66 (s, 1H, H₄), 9.80 (s, 1H, OH), 10.11 (s, 1H, OH); ¹³C NMR (100 MHz, DMSO-d₆) δ: 114.4 (CH), 115.6 (2CH), 116.4 (CH), 118.5 (CH), 127.8 (Cq), 130.3 (2CH), 130.5 (CH), 131.7 (CH), 137.1 (Cq), 137.5 (Cq), 140.6 (Cq), 146.4 (Cq), 150.8 (CH), 158.2 (Cq), 160.6 (Cq), 160.9 (Cq), 161.3 (CH); HRMS (EI-MS): C₁₉H₁₃N₃O₂, calculated m/z 316.1086 (M+1). found m/z 316.1079 (M+1).

### 2-(4-Hydroxyphenyl)-7-(2-hydroxyphenyl)-pyrido[3,2-d]pyrimidine (24)

The product 24 is synthesized from 17 by following the general procedure B for 15 min and then isolated after purification on a chromatographic silica gel column (Acetone/CH₂Cl₂, 05/95) as a yellow solid with a yield of 66%. MP: 278-279° C.; IR (ATR, Diamond, cm⁻¹) ν 3109, 2177, 1595, 1548, 1454, 1383, 1280, 1162, 959, 821; ¹H NMR (250 MHz, DMSO-d₆) δ: 6.95 (d, 2H, J=14.0 Hz, H_{Arom}), 6.97-7.08 (m, 2H, H_{Arom}), 7.30-7.37 (m, 1H, H_{Arom}), 7.57-7.60 (m, 1H, H_{Arom}), 8.43-8.46 (m, 2H, H_{Arom} and H₈), 9.24 (d, 1H, J=3.2 Hz, H₆), 9.64 (s, 1H, H₄), 10.10 (sl, 2H, OH); ¹³C NMR (62.5 MHz, DMSO-d₆) δ: 115.6 (2CH), 116.4 (CH), 120.0 (CH), 123.0 (Cq), 127.9 (Cq), 130.3 (2CH), 130.7 (CH), 130.9 (CH), 133.9 (CH), 136.8 (Cq), 139.6 (Cq), 146.4 (Cq), 152.8 (CH), 155.0 (Cq), 160.5 (Cq), 160.6 (Cq), 161.1 (CH); HRMS (EI-MS): C₁₉H₁₃N₃O₂, calculated m/z 316.1086 (M+1). found m/z 316.1078 (M+1).

### 2-(4-Hydroxyphenyl)-7-(4-trifluoromethyl phenyl)-pyrido[3,2-d]pyrimidine (25)

The product 25 is synthesized from 17 by following the general procedure B pendant 15 min and then isolated after purification on a chromatographic silica gel column (acetone/petroleum ether, 10/90) as a yellow solid with a yield of 76%. MP: 203-204° C.; IR (ATR, Diamond, cm⁻¹) ν 3606, 3037, 1604, 1573, 1461, 1399, 1321, 1109, 1066, 838; ¹H NMR (400 MHz, DMSO-d₆) δ: 6.95 (d, 2H, J=8.8 Hz, H_{Arom}), 7.93 (d, 2H, J=8.3 Hz, H_{Arom}), 8.22 (d, 2H, J=8.3 Hz, H_{Arom}), 8.44 (d, 2H, J=8.8 Hz, H_{Arom}), 8.69 (d, 1H, J=2.1 Hz, H₈), 9.40 (d, 1H, J=2.1 Hz, H₆), 9.68 (s, 1H, H₄), 10.11 (s, 1H, OH); ¹³C NMR (100 MHz, DMSO-d₆) δ: 115.3 (CH), 126.0 (Cq, J_{C-F}=253 Hz), 126.4 (CH, J_{C-F}=3.8 Hz), 128.5 (Cq), 129.1 (CH), 130.1 (Cq, J_{C-F}=29 Hz), 130.9 (CH), 132.5 (CH), 137.6 (Cq), 138.6 (Cq), 139.5 (Cq), 145.9 (Cq), 150.0 (CH), 160.2 (2Cq), 160.9 (CH); HRMS (EI-MS): C₂₀H₁₂F₃N₃O, calculated m/z 368.1011 (M+1). found m/z 368.1009 (M+1).

### 2-(4-Hydroxyphenyl)-7-(4-methanesulfonylphenyl)-pyrido[3,2-d]pyrimidine (26)

The product 26 is synthesized from 17 by following the general procedure B for 15 min and then isolated after purification on a chromatographic silica gel column (acetone/petroleum ether, 10/90) as a yellow solid with a yield of 89%. MP: 306-307° C.; IR (ATR, Diamond, cm⁻¹) ν 3365, 3022, 1604, 1584, 1456, 1394, 1271, 1143, 1087, 949, 841; ¹H NMR (400 MHz, DMSO-d₆) δ: 3.32 (s, 3H, CH₃), 6.95 (d, 2H, J=14.0 Hz, H_{Arom}), 8.12 (d, 2H, J=13.5 Hz, H_{Arom}), 8.30 (d, 2H, J=13.5 Hz, H_{Arom}), 8.45 (d, 2H, J=14.0 Hz, H_{Arom}), 8.74 (d, 1H, J=2.2 Hz, H₈), 9.43 (d, 1H, J=2.2 Hz, H₆), 9.71 (s, 1H, H₄), 10.13 (s, 1H, OH); ¹³C NMR (100 MHz, DMSO-d₆) δ: 43.4 (CH₃), 115.7 (2CH), 127.7 (Cq), 127.8 (2CH), 128.9 (2CH), 130.4 (2CH), 133.3 (CH), 137.9 (Cq), 138.8 (Cq), 140.7 (Cq), 141.2 (Cq), 146.2 (Cq), 150.7 (CH), 160.7 (Cq), 161.0 (Cq), 161.5 (CH); HRMS (EI-MS): C₂₀H₁₈N₃O₃S, calculated m/z 378.0912 (M+1). found m/z 378.0919 (M+1).

### 2-(4-Hydroxyphenyl)-7-(4-mercaptophenyl)-pyrido[3,2-d]pyrimidine (27)

The product 27 is synthesized from 17 by following the general procedure B for 15 min and then isolated after purification on a chromatographic silica gel column (MeOH/CH₂Cl₂, 02/98) as a yellow solid with a yield of 70%. MP: 222-223° C.; IR (ATR, Diamond, cm⁻¹) ν 3114, 2254, 1578, 1538, 1449, 1368, 1269, 1160, 1072, 954, 841; ¹H NMR (400 MHz, DMSO-d₆) δ: 6.87 (d, 2H, J=8.7 Hz, H_{Arom}), 7.58-7.62 (m, 4H, H_{Arom}), 7.67-7.69 (m, 2H, H₈ and SH), 8.33 (d, 2H, J=8.7 Hz, H_{Arom}), 8.82 (d, 1H, J=2.0 Hz, H₆), 9.53 (s, 1H, H₄), 10.08 (sl, 1H, OH); ¹³C NMR (100 MHz, DMSO-d₆) δ: 115.6 (2CH), 127.6 (Cq), 129.1 (Cq), 130.1 (CH), 130.2 (CH), 130.4 (2CH), 130.5 (2CH), 134.4 (2CH), 136.2 (Cq), 142.1 (Cq), 146.3 (Cq), 149.9 (CH), 160.6 (2Cq), 161.0 (CH), 161.1 (Cq); HRMS (EI-MS): C₁₈H₁₃N₃OS, calculated m/z 332.0858 (M+1). found m/z 332.0864 (M+1).

### 2-(4-Hydroxyphenyl)-7-(4-cyanophenyl)-pyrido[3,2-d]pyrimidine (28)

The product 28 is synthesized from 17 by following the general procedure B for 15 min and then isolated after purification on a chromatographic silica gel column (MeOH/CH₂Cl₂, 5/95) as a yellow solid with a yield of 83%. MP: 313-314° C.; IR (ATR, Diamond, cm⁻¹) ν 3386, 2234, 1604, 1548, 1461, 1399, 1236, 1159, 959, 837; ¹H NMR (400 MHz, DMSO-d₆) δ: 6.95 (d, 2H, J=13.4 Hz, H_{Arom}), 8.05 (d, 2H, J=12.8 Hz, H_{Arom}), 8.22 (d, 2H, J=12.8 Hz, H_{Arom}), 8.43 (d, 2H, J=13.4 Hz, H_{Arom}), 8.71 (sl, 1H, H₈), 9.40 (sl, 1H, H₆), 9.68 (s, 1H, H₄), 10.12 (s, 1H, OH); ¹³C NMR (100 MHz, DMSO-d₆) δ: 111.9 (Cq), 115.7 (2CH), 118.6 (Cq), 127.7 (Cq), 128.8 (2CH), 130.4 (2CH), 133.1 (2CH), 133.2 (CH), 137.9 (Cq), 138.6 (Cq), 140.3 (Cq), 146.2 (Cq), 150.6 (CH), 160.7 (Cq), 161.0 (Cq), 161.5 (CH); HRMS (EI-MS): C₂₀H₁₂N₄O, calculated m/z 325.1089 (M+1). found m/z 325.1094 (M+1).

### 2-(4-Hydroxyphenyl)-7-(3-cyanophenyl)-pyrido[3,2-d]pyrimidine (29)

The product 29 is synthesized from 17 by following the general procedure B for 15 mins and then isolated after purification by recrystallization from methanol as a green solid with a yield of 77%. MP: >268° C.; IR (ATR, Diamond, cm⁻¹) ν 3116, 2230, 1580, 1462, 1370, 1244, 1161, 805; ¹H NMR (400 MHz, DMSO-d₆) δ: 6.93 (d, 2H, J=8.6 Hz, H_{Arom}), 7.78 (t, 1H, J=7.8 Hz, H_{Arom}), 7.98 (d, 1H, J=7.6 Hz, H_{Arom}), 8.35 (d, 1H, J=7.9 Hz, H_{Arom}), 8.42 (d, 2H, J=8.6 Hz, H_{Arom}), 8.54 (s, 1H, H_{Arom}), 8.73 (d, 1H, J=1.5 Hz, H₈), 9.41 (d, 1H, J=1.9 Hz, H₆), 9.68 (s, 1H, H₄), 10.19 (broad, 1H, OH); ¹³C NMR (100 MHz, DMSO-d₆) δ: 112.4 (Cq), 115.6 (2CH), 118.4 (Cq), 127.4 (Cq), 130.2 (2CH), 130.3 (CH), 131.4 (CH), 132.5 (CH), 132.6 (CH), 132.7 (CH), 136.8 (Cq), 137.7 (Cq), 138.2 (Cq), 146.1 (Cq), 150.4 (CH), 160.8 (Cq), 160.9 (Cq), 161.3 (CH); HRMS (EI-MS): C₂₀H₁₂N₄O, calculated m/z 325.1089 (M+1). found m/z 325.1084 (M+1).

### 2-(3-Hydroxyphenyl)-7-(4-hydroxyphenyl)-pyrido[3,2-d]pyrimidine (30)

The product 30 is synthesized from 18 by following the general procedure B pendant 15 min and then isolated after purification on a chromatographic silica gel column (CH₂Cl₂/NH₃ 99/1, CH₂Cl₂/MeOH, 99/1) as a brown solid with a yield of 67%. MP:>268° C.; IR (ATR, Diamond, cm⁻¹) ν 3344, 1577, 1549, 1459, 1363, 1236, 1236, 1173, 955, 828; ¹H NMR (400 MHz, DMSO-d₆) δ: 6.96 (m, 3H), 7.37 (t, 1H, J=8.0 Hz, H_{Arom}), 7.87 (d, 2H, J=8.0 Hz, H_{Arom}), 8.02 (d, 2H, J=4.0 Hz, H_{Arom}), 8.51 (s, 1H, H₈), 9.40 (s, 1H, H₆), 9.66 (s, 1H, H₄), 9.83 (broad, 2H, OH); ¹³C NMR (400 MHz, DMSO-d₆) δ: 115.0 (CH), 116.3 (2CH), 118.2 (CH), 119.2 (CH), 126.1 (Cq), 129.2 (2CH), 129.8 (CH), 130.0 (CH), 137.2 (Cq), 138.3 (Cq), 140.5 (Cq), 146.6 (Cq), 151.4 (CH), 157.8 (Cq), 159.0 (Cq), 160.6 (Cq), 161.1 (CH). HRMS (EI-MS): C₁₉H₁₃N₃O₂, calculated m/z 316.1086 (M+1). found m/z 316.1101 (M+1).

### 2-(2-Hydroxyphenyl)-7-(4-hydroxyphenyl)-pyrido[3,2-d]pyrimidine (31)

The product 31 is synthesized from 19 by following the general procedure B pendant 15 min and then isolated by purification on a chromatographic silica gel column (CH₂Cl₂/NH₃ 99/1, CH₂Cl₂/MeOH, 99.5/0.5) as a brown solid with a yield of 65%. MP: >268° C.; IR (ATR, Diamond, cm⁻¹) ν 3262, 2923, 1589, 1548, 1468, 1371, 1227, 1176, 826, 761; ¹H NMR (400 MHz, DMSO-d₆) δ: 6.97-7.08 (m, 4H, H_{Arom}), 7.47-7.51 (m, 1H, H_{Arom}), 7.93-7.97 (d, 2H, J=16.0 Hz, H_{Arom}), 8.58-8.62 (dd, 1H, J=4.0 Hz, J=16.0 Hz, H_{Arom}), 8.75 (d, 1H, J=4.0 Hz, H₈), 9.47 (s, 1H, H₆), 9.78 (s, 1H, H₄), 10.03 (s, 1H, OH), 13.41 (s, 1H, OH); ¹³C NMR (400 MHz, DMSO-d₆) δ: 116.2 (2CH), 117.7 (CH), 118.4 (Cq), 119.0 (CH), 125.7 (Cq), 129.3 (2CH), 129.5 (CH), 131.4 (CH), 131.5 (CH), 132.0 (Cq), 136.4 (Cq), 141.0 (Cq), 144.8 (Cq), 151.4 (CH), 159.2 (Cq), 160.2 (Cq), 161.3 (CH). C₁₉H₁₃N₃O₂, calculated m/z 316.1086 (M+1). found m/z 316.1103 (M+1).

### 2-(4-Hydroxyphenyl)-7-(3-(6-methoxy)-pyridinyl)-pyrido[3,2-d]pyrimidine (32)

The product 32 is synthesized from 17 by following the general procedure B for 10 mins and then isolated after purification on a chromatographic silica gel column (CH₂Cl₂/MeOH, 98/2 yield of 82%) as a greenish solid with MP: 246-248° C.; IR (ATR, Diamond, cm⁻¹) ν 3420, 1572, 1461, 1397, 1273, 1156, 828, 699; ¹H NMR (400 MHz, DMSO-d₆) δ: 3.93 (s, 3H, CH₃), 6.92 (d, 2H, J=8.7 Hz, H_{3′}), 6.98 (d, 1H, J=8.7 Hz, H_{Pyr5}), 8.33 (dd, 1H, J=2.5 Hz, J=8.7 Hz, H_{Pyr4}), 8.40 (d, 2H, J=8.6 Hz, H₂), 8.59 (d, 1H, J=1.4 Hz, H₈), 8.81 (d, 1H, J=2.5 Hz, H_{Pyr2}), 9.36 (d, 1H, J=1.9 Hz, H₆), 9.62 (s, 1H, H₄), 10.10 (s, 1H, OH); ¹³C NMR (100 MHz, DMSO-d₆) δ: 53.4 (CH₃), 111.0 (CH), 115.0 (2CH), 125.0 (Cq), 127.7 (Cq), 130.2 (2CH), 131.0 (CH), 137.2 (Cq), 137.5 (Cq), 138.3 (CH), 146.2 (CH), 146.3 (Cq), 150.3 (CH), 160.5 (Cq), 160.8 (Cq), 161.9 (CH), 164.1 (Cq). C₁₉H₁₄N₄O₂, calculated m/z 331.1195 (M+1). found m/z 331.1184 (M+1).

### 2-(4-Hydroxyphenyl)-7-(3-pyridinyl)-pyrido[3,2-d]pyrimidine (33)

The product 33 is synthesized from 17 by following the general procedure B for 10 mins and then isolated after purification on a chromatographic silica gel column (CH₂Cl₂/MeOH, 98/2) as a yellow solid with a yield of 88%. MP: >268° C.; IR (ATR, Diamond, cm⁻¹) ν 2919, 1580, 1458, 1339, 1247, 1160, 811; ¹H NMR (400 MHz, DMSO-d₆) δ: 6.92 (d, 2H, J=8.7 Hz, H_{3′}), 7.57 (dd, 1H, J=4.7 Hz, J=7.9 Hz, H_{Pyr4}), 8.40-8.44 (m, 3H, H_{Arom}), 8.69-8.73 (m, 2H, H_{Py2} H_{Pyr6}), 9.19 (d, 1H, J=1.9 Hz, H₈), 9.39 (d, 1H, J=2.1 Hz, H₆), 9.66 (s, 1H, H₄), 10.11 (s, 1H, OH); ¹H NMR (100 MHz, DMSO-d₆) δ: 115.5 (2CH), 124.0 (CH), 127.6 (Cq), 130.2 (2CH), 131.4 (Cq), 132.5 (CH), 135.2 (CH), 137.5 (Cq), 137.6 (Cq), 146.16 (Cq), 148.5 (CH), 150.0 (CH), 150.5 (CH), 160.5 (Cq), 160.8 (Cq), 161.3 (CH). C₁₈H₁₂N₄O, calculated m/z 301.1089 (M+1). found m/z 301.1075 (M+1).

### 2-(4-Hydroxyphenyl)-7-(4-pyridinyl)-pyrido[3,2-d]pyrimidine (34)

The product 34 is synthesized from 17 by following the general procedure B for 10 mins and then isolated after purification on a chromatographic silica gel column (CH₂Cl₂/NH₃ 99/1, CH₂Cl₂/MeOH, 98/2) as a yellow solid with a yield of 77%. MP: >268° C.; IR (ATR, Diamond, cm⁻¹) ν 2917, 1570, 1457, 1368, 1251, 1156, 809, 721; ¹H NMR (400 MHz, DMSO-d₆) δ: 6.94 (d, 2H, J=8.8 Hz, H_{3′}), 8.04 (d, 2H, J=5.8 Hz, H_{Pyr3}), 8.42 (d, 2H, J=8.7 Hz, H_{2′}), 8.78 (d, 3H, J=1.4 Hz, H_{Arom}), 9.44 (d, 1H, J=2.1 Hz, H₆), 9.70 (s, 1H, H₄), 10.15 (s, 1H, OH); ¹³C NMR (100 MHz, DMSO-d₆) δ: 115.6 (2CH), 122.0 (2CH), 127.5 (Cq), 130.3 (2CH), 133.2 (CH), 137.5 (Cq), 138.1 (Cq), 142.8 (Cq), 146.1 (Cq), 150.2 (CH), 150.4 (2CH), 160.6 (Cq), 160.9 (Cq), 161.5 (CH). C₁₈H₁₂N₄O, calculated m/z 301.1089 (M+1). found m/z 301.1075 (M+1).

### 2-(4-Hydroxyphenyl)-7-(2-furyl)-pyrido[3,2-d]pyrimidine (35)

The product 35 is synthesized from 17 by following the general procedure B for 10 mins and then isolated after purification on a chromatographic silica gel column (CH₂Cl₂/NH₃ 99/1, CH₂Cl₂/MeOH, 98/2) as a pale brown solid with a yield of 70%. MP: 261-263° C.; IR (ATR, Diamond, cm⁻¹) ν 3012, 1581, 1378, 1234, 1167, 805, 759; ¹H NMR (400 MHz, DMSO-d₆) δ: 6.78 (dd, 1H, J=1.7 Hz, J=3.4 Hz, H_{Fur4}), 6.93 (d, 2H, J=8.7, H_{3′}), 7.57 (d, 1H, J=3.4 Hz, H_{Fur5}), 8.01 (d, 1H, J=1.1, H_{Fur3}), 8.41 (d, 2H, J=8.7 Hz, H₂), 8.46 (d, 1H, J=1.5 Hz, H₈), 9.41 (d, 1H, J=1.9 Hz, H₆), 9.58 (s, 1H, H₄), 10.10 (s, 1H, OH); ¹³C NMR (100 MHz, DMSO-d₆) δ: 111.2 (CH), 112.8 (CH), 115.5 (2CH), 126.6 (CH), 127.7 (Cq), 130.2 (2CH), 130.3 (Cq), 137.0 (Cq), 145.5 (CH), 146.4 (Cq), 147.8 (CH), 149.3 (Cq), 160.5 (Cq), 160.8 (CH), 161.9 (Cq). C₁₇H₁₁N₃O₂, calculated m/z 290.0930 (M+1). found m/z 290.0928 (M+1).

### 2-(4-Hydroxyphenyl)-7-(3-furyl)-pyrido[3,2-d]pyrimidine (36)

The product 36 is synthesized from 17 by following the general procedure B for 10 mins and then isolated after purification on a chromatographic silica gel column (CH₂Cl₂/MeOH, 95/5) as a yellow solid with a yield of 70%. MP: >268° C.; IR (ATR, Diamond, cm⁻¹) ν 3061, 1590, 1440, 1272, 1161, 823, 696; ¹H NMR (400 MHz, DMSO-d₆) δ: 6.92 (d, 2H, J=8.7 Hz, H_{3′}), 7.37 (d, 1H, J=1.1 Hz, H_{Fur4}), 7.89 (d, 1H, J=1.5, H_{Fur5}), 8.39 (d, 2H, J=8.7 Hz, H₂), 8.56 (d, 1H, J=1.4 Hz, H₈), 8.68 (s, 1H, H_{Fur2}), 9.36 (d, 1H, J=2.0 Hz, H₆), 9.58 (s, 1H, H₄), 10.09 (s, 1H, OH); ¹³C NMR (400 MHz, DMSO-d₆) δ: 108.6 (CH), 115.54 (2CH), 122.2 (Cq), 127.8 (Cq), 129.3 (CH), 130.1 (2CH), 133.1 (Cq), 137.1 (Cq), 142.3 (CH), 145.1 (CH), 146.7 (Cq), 150.0 (CH), 160.4 (Cq), 160.7 (Cq), 160.9 (CH). C₁₇H₁₁N₃O₂, calculated m/z 290.0930 (M+1). found m/z 290.0919 (M+1).

### 2-(4-Hydroxyphenyl)-7-(3-thiophyl)-pyrido[3,2-d]pyrimidine (37)

The product 37 is synthesized from 17 by following the general procedure B for 10 mins and then isolated after purification on a chromatographic silica gel column (CH₂Cl₂/NH₃ 99/1, CH₂Cl₂/MeOH, 98/2) as a yellow solid with a yield of 65%. MP: 248-250° C.; IR (ATR, Diamond, cm⁻¹) ν 3386, 2234, 1604, 1548, 1461, 1399, 1236, 1159, 959, 837; ¹H NMR (400 MHz, DMSO-d₆) δ: 6.94 (d, 2H, J=8.6 Hz, H_{3′}), 7.80 (d, 1H, J=2.7 Hz, Hₜₕ) 7.94 (d, 1H, J=5.0 Hz, H_{Thiop5}), 8.41 (d, 2H, J=8.62 Hz, H_{2′}), 8.48 (d, 1H, J=1.3 Hz, H_{Thiop2}), 8.65 (s, 1H, H₈), 9.48 (s, 1H, H₆), 9.59 (s, 1H, H₄), 10.12 (s, 1H, OH).

### 2-(4-Hydroxyphenyl)-7-(2-thiophyl)-pyrido[3,2-d]pyrimidine (38)

The product 38 is synthesized from 17 by following the general procedure B for 10 mins and then isolated after purification on a chromatographic silica gel column (CH₂Cl₂/NH₃ 99/1, CH₂Cl₂/MeOH, 98/2) as a yellow solid with a yield of 63%. MP: 240-242° C.; IR (ATR, Diamond, cm⁻¹) ν 3066, 1584, 1460, 1371, 1161, 847, 693; ¹H NMR (400 MHz, DMSO-d₆) δ: 6.95 (d, 2H, J=8.7 Hz, H_{3′}), 7.31 (dd, 1H, J=4.0 Hz, J=4.7 Hz, H_{Thiop4}), 7.87 (d, 1H, J=5.2 Hz, H_{Thiop3}), 8.08 (d, 1H, J=3.7 Hz, H_{Thiop5}), 8.44 (d, 2H, J=8.7 Hz, H_{2′}), 8.52 (d, 1H, J=2.1 Hz, H₈), 9.42 (d, 1H, J=2.1 Hz, H₆), 9.63 (s, 1H, H₄), 10.12 (s, 1H, OH).

### 2-(4-Hydroxyphenyl)-7-(3-(4-formyl)thiophyl)-pyrido[3,2-d]pyrimidine (39)

The product 39 is synthesized from 17 by following the general procedure B pendant 8 min and then isolated after purification on a chromatographic silica gel column (CH₂Cl₂/MeOH, 98/2) as a yellow solid with a yield of 68%. MP: 240-242° C.; IR (ATR, Diamond, cm⁻¹) ν 3066, 1584, 1460, 1371, 1161, 847, 693; ¹H NMR (400 MHz, DMSO-d₆) δ: 6.93 (d, 2H, J=12.0 Hz, H_{Arom}), 8.09 (d, 1H, J=4.0 Hz, H_{Thiop}), 8.41 (m, 3H, H_{Arom}+H_{Thiop}), 8.83 (s, 1H, H₈), 9.06 (s, 1H, H₆), 9.65 (s, 1H, H₄), 9.99 (s, 1H, CHO), 10.1 (s, 1H, OH); ¹³C NMR (400 MHz, DMSO-d₆) δ: 115.6 (2CH), 127.7 (Cq), 130.2 (CH), 130.3 (2CH), 134.0 (CH), 135.8 (Cq), 136.2 (Cq), 137.2 (Cq), 139.0 (Cq), 142.4 (CH), 146.0 (Cq), 152.3 (CH), 160.5 (Cq), 160.7 (Cq), 161.2 (CH), 186.0 (CH). C₁₈H₁₁N₃O₂S, calculated m/z 334.0650 (M+1). found m/z 334.0666 (M+1).

### 2-(4-Hydroxyphenyl)-7-(3-(5-formyl)thiophyl)-pyrido[3,2-d]pyrimidine (40)

The product 40 is synthesized from 17 by following the general procedure B for 10 mins and then isolated after purification on a chromatographic silica gel column (CH₂Cl₂/MeOH, 98/2) as a pale brown solid with a yield of 69%. MP: >268° C.; IR (ATR, Diamond, cm⁻¹) ν 3262, 2923, 1589, 1548, 1468, 1371, 1227, 1176, 826, 761; ¹H NMR (400 MHz, DMSO-d₆) δ: 6.92 (d, 2H, J=8.0 Hz, H_{Arom}), 7.54 (m, 1H, H_{Thiop5}), 8.38 (d, 2H, J=8.0 Hz, H_{Arom}), 8.67 (s, 1H, H₈), 8.80 (s, 1H, H₆), 8.95 (s, 1H, H_{Thio2}), 9.43 (d, 1H, J=4.0 Hz, H₄), 9.57 (s, 1H, CHO), 10.00 (s, 1H, OH); ¹³C NMR (400 MHz, DMSO-d₆) δ: 115.6 (2CH), 127.7 (Cq), 130.2 (2CH), 130.8 (CH), 134.0 (Cq), 134.1 (CH), 136.4 (CH), 137.4 (Cq), 138.1 (Cq), 144.4 (Cq), 146.4 (Cq), 149.9 (CH), 160.6 (Cq), 160.9 (Cq), 161.1 (CH), 184.1 (CH). C₁₈H₁₁N₃O₂S, calculated m/z 334.0650 (M+1). found m/z 334.0640 (M+1).

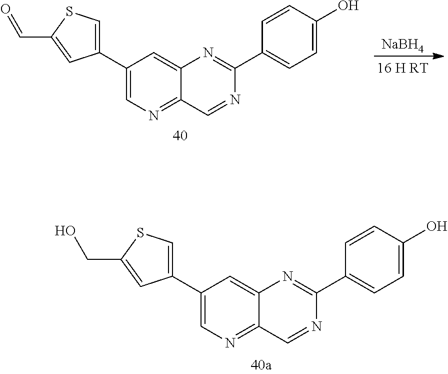 [SMILES: O=Cc1cc(-c2cnc3cnc(-c4ccc(O)cc4)nc3c2)cs1 ; "NaBH4" ; OCc1cc(-c2cnc3cnc(-c4ccc(O)cc4)nc3c2)cs1]

### 2-(4-Hydroxyphenyl)-7-(3-(5-hydroxymethyl)thiophyl)-pyrido[3,2-d]pyrimidine (40a)

In a 25 mL flask, 203 mg (0.60 mmol, 1 equiv.) of 2-(4-hydroxyphenyl)-7-(3-(5-formyl)thiophyl)-pyrido[3,2-c]pyrimidine (40) are dissolved in 10 mL of a mixture of solvants MeOH/DMF 1/1. After a few minutes of stirring at room temperature, the mixture is cooled to −10° C. At this temperature, sodium borohydride (11.51 mg, 0.3 mmol, 0.5 equiv.) is added. The whole is left with stirring at room temperature overnight. The solvants are then evaporated and the thereby obtained residue is purified on a chromatographic silica gel column (DCM/MeOH, from 10/0 to 9/1). The product X6 is obtained as a while solid, with a yield of 54%. It may also be obtained from 40a by treatment with 10% aqueous hydrochloric acid in MeOH with a quantative yield. MP>268° C.; IR (ATR, Diamond, cm⁻¹) ν 3353, 3077, 1595, 1452, 1236, 1148, 999, 840, 739; ¹H NMR (400 MHz, DMSO-d₆) δ: 4.73 (s, 2H, CH₂), 5.65 (s, 1H, OH), 6.93 (d, 2H, J=8.0 Hz, H_{Arom}), 7.76 (s, 1H, H_{Thiop5}), 8.36 (s, 1H, H_{Thiop2}), 8.41 (d, 2H, J=12.0 Hz, H_{Arom}), 8.58 (5, 1H, H₈), 9.44 (s, 1H, H₆), 9.59 (s, 1H, H₄), 10.11 (s, 1H, OH); ¹³C NMR (100 MHz, DMSO-d₆) δ: 58.3 (CH₂), 115.6 (2CH), 122.9 (CH), 124.5 (CH), 127.8 (Cq), 129.8 (CH), 130.2 (2CH), 135.5 (Cq), 136.2 (Cq), 137.2 (Cq), 146.8 (Cq), 148.6 (Cq), 150.3 (CH), 160.5 (Cq), 160.8 (Cq), 161.0 (CH). C₁₈H₁₃N₃O₂S, calculated m/z 336.0804 (M+1). found 336.0801 (M+1).

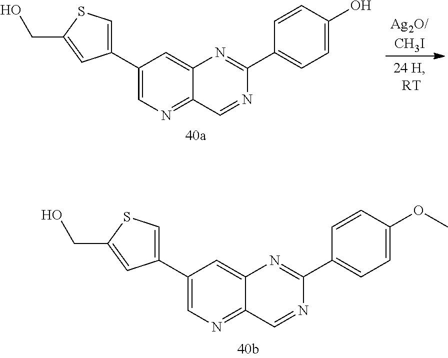 [SMILES: OCc1cc(-c2cnc3cnc(-c4ccc(O)cc4)nc3c2)cs1 ; "Ag2O/CH3I" ; COc1ccc(-c2ncc3ncc(-c4csc(CO)c4)cc3n2)cc1]

### 2-(4-Hydroxyphenyl)-7-(3-(5-methoxymethyl)thiophyl)-pyrido[3,2-d]pyrimidine (40b)

In a 25 mL flask, 100 mg (0.29 mmol, 1 equiv.) of 2-(4-hydroxyphenyl)-7-(3-(5-hydroxymethyl)thiophyl)-pyrido[3,2-c]pyrimidine (40a) are dissolved in 12 mL of THF. To this mixture are added silver oxide (186 μL, 2.98 mmol, 10.0 equiv.) and iodomethane (277 mg, 1.19 mmol, 4.0 equiv.). The whole is left with stirring at room temperature for 24 hours. Next, the solvants are evaporated and the thereby obtained residue is purified on a chromatographic silica gel column (DCM/MeOH, 98/02). The product 40b is obtained as a yellow solid, with a yield of 71%. MP: 251° C.; IR (ATR, Diamond, cm⁻¹) ν 3274, 1935, 1677, 1593, 1440, 1247, 1164, 1020, 841, 659; ¹H NMR (400 MHz, CDCl₃) δ: 1.41 (s, 1H, OH), 3.90 (s, 3H, OCH₃), 4.92 (d, 2H, J=4.0 Hz, CH₂), 7.03 (d, 2H, J=8.0 Hz, H_{Arom}), 7.47 (s, 1H, Hₜₕᵢₒₚ), 7.74 (s, 1H, Hₜₕᵢₒₚ), 8.38 (s, 1H, H₈), 8.56 (d, 2H, J=8.0 Hz, H_{Arom}), 9.20 (s, 1H, H₆), 9.58 (s, 1H, H₄); ¹³C NMR (100 MHz, DMSO-d₆) δ: 55.3 (OCH₃), 58.3 (CH₂), 114.4 (2CH), 122.8 (CH), 124.4 (CH), 129.3 (Cq), 129.7 (CH), 130.0 (2CH), 135.4 (Cq), 136.2 (Cq), 137.1 (Cq), 146.7 (Cq), 148.5 (Cq), 150.5 (CH), 160.4 (Cq), 160.9 (CH), 161.7 (Cq). C₁₃H₁₅N₃O₂S, calculated m/z 350.0960 (M+1). found 350.0957 (M+1).

### 2-(4-Hydroxyphenyl)-7-(4-hydrohymethyl)-pyrido[3,2-d]pyrimidine (41)

The product 41 is synthesized from 17 by following the general procedure B for 10 mins and then isolated after purification on a chromatographic silica gel column (CH₂Cl₂/acetone, 95/05) as a brown solid with a yield of 75%. MP: 262-264° C.; IR (ATR, Diamond, cm⁻¹) ν 3087, 2363, 1671, 1571, 1438, 1362, 1275, 1160, 827, 737; ¹H NMR (400 MHz, DMSO-d₆) δ: 4.60 (d, 2H, J=4.0 Hz, CH₂), 5.34 (t, 1H, J=4.0 Hz, OH), 6.94 (d, 2H, J=8.0 Hz, H_{Arom}), 7.52 (d, 2H, J=8.0 Hz, H_{Arom}), 7.96 (d, 2H, J=8.0 Hz, H_{Arom}), 8.43 (m, 2H, H_{Arom}), 8.57 (s, 1H, H₈), 9.37 (s, 1H, H₆), 9.65 (s, 1H, H₄), 10.11 (s, 1H, OH); ¹³C NMR (100 MHz, DMSO-d₆) δ: 62.4 (CH₂), 115.6 (2CH), 127.2 (2CH), 127.5 (2CH), 127.8 (Cq), 130.3 (2CH), 131.4 (CH), 134.0 (Cq), 137.4 (Cq), 140.3 (Cq), 144.0 (Cq), 146.5 (Cq), 150.7 (CH), 160.5 (Cq), 160.8 (Cq), 161.2 (CH). C₂₀H₁₅N₃O₂, calculated m/z 330.1243 (M+1). found m/z 330.1251 (M+1).

### 2-(4-Hydroxyphenyl)-7-(4-formylphenyl)-pyrido[3,2-d]pyrimidine (42)

The product 42 is synthesized from 17 by following the general procedure B for 8 mins and then isolated after purification on a chromatographic silica gel column (CH₂Cl₂/NH₃ 99/01, CH₂Cl₂/Methanol 99/01) as a yellow solid with a yield of 66%. MP: >268° C.; IR (ATR, Diamond, cm⁻¹) ν 3374, 2985, 2196, 1656, 1569, 1441, 1253, 1105, 845, 797, 697; ¹H NMR (400 MHz, DMSO-d₆) δ: 6.94 (d, 2H, J=8.0 Hz, H_{Arom}), 8.08 (d, 2H, J=8.0 Hz, H_{Arom}), 8.22 (d, 2H, J=8.0 Hz, H_{Arom}), 8.42 (d, 2H, J=12.0 Hz, H_{Arom}), 8.70 (s, 1H, H₈), 9.42 (s, 1H, H₆), 9.68 (s, 1H, H₄), 10.12 (m, 2H, OH+CHO); ¹³C NMR (100 MHz, DMSO-d₆) δ: 115.6 (2CH), 127.7 (Cq), 128.6 (2CH), 130.2 (2CH), 130.3 (2CH), 133.0 (CH), 136.2 (Cq), 137.8 (Cq), 139.1 (Cq), 141.2 (Cq), 146.2 (Cq), 150.6 (CH), 160.6 (Cq), 160.9 (Cq), 161.4 (CH), 192.8 (CH). C₂₀H₁₃N₃O₂, calculated m/z 328.1086 (M+1). found m/z 328.1078 (M+1).

### 2-(4-hydroxyphenyl)-7-(2-methoxy-4-hydroxyphenyl)-pyrido[3,2-d]pyrimidine (43)

The product 43 is synthesized from 17 by following the general procedure B for 15 mins and then isolated after purification on a chromatographic silica gel column (CH₂Cl₂/Methanol 98/02) as a yellow solid with a yield of 55%. MP: 207-208° C.; IR (ATR, Diamond, cm⁻¹) ν 30100, 2925, 1706, 1574, 1453, 1366, 1161, 847, 691; ¹H NMR (400 MHz, DMSO-d₆) δ: 3.94 (s, 3H, OCH₃), 6.93-6.97 (m, 3H, H_{Arom}), 7.45 (d, 1H, J=8 Hz, H_{Arom}), 7.56 (s, 1H, H_{Arom}), 8.42 (d, 2H, J=8 Hz, H_{Arom}), 8.53 (s, 1H, H₈), 9.37 (s, 1H, H₆), 9.59 (s, 1H, H₄), 9.82 (s, broad, 2H, OH); ¹³C NMR (100 MHz, DMSO-d₆) δ: 55.8 (OCH₃), 111.5 (CH), 115.6 (2CH), 116.1 (CH), 120.7 (CH), 126.6 (Cq), 127.9 (Cq), 130.1 (CH), 130.2 (2CH), 136.9 (Cq), 140.5 (Cq), 146.7 (Cq), 148.2 (Cq), 148.4 (Cq), 150.8 (CH), 160.4 (Cq), 160.7 (Cq), 160.9 (CH). C₂₈H₁₈N₃O₃, calculated m/z 346.1190 (M+1). found m/z 346.1186 (M+1).

### 2-(4-Hydroxyphenyl)-7-(3-formylphenyl)-pyrido[3,2-d]pyrimidine (44)

The product 44 is synthesized from 17 by following the general procedure B for 10 mins and then isolated after purification on a chromatographic silica gel column (CH₂Cl₂/Et₃N 99/01, CH₂Cl₂/Methanol 99.5/0.5) as a yellow solid with a yield of 80%. MP: 266° C.; IR (ATR, Diamond, cm⁻¹) ν 3292, 3029, 1683, 1583, 1458, 1399, 1275, 1158, 811, 733, 692; ¹H NMR (400 MHz, DMSO-d₆) δ: 6.95 (d, 2H, J=8.0 Hz, H_{Arom}), 7.82 (t, 1H, J=8.0 Hz, H_{Arom}), 8.04 (d, 1H, J=8.0 Hz, H_{Arom}), 8.36 (d, 1H, J=8.0 Hz, H_{Arom}), 8.44 (d, 2H, J=8.0 Hz, H_{Arom}), 8.57 (s, 1H, H_{Arom}), 8.73 (s, 1H, H₈), 9.46 (s, 1H, H₆), 9.70 (s, 1H, H₄), 10.11 (s, 1H, OH), 10.16 (s, 1H, CHO); ¹³C NMR (100 MHz, DMSO-d₆) δ: 115.6 (2CH), 127.7 (Cq), 129.2 (CH), 129.7 (CH), 130.2 (CH), 130.3 (2CH), 132.5 (CH), 133.6 (CH), 136.6 (Cq), 137.0 (Cq), 137.7 (Cq), 139.2 (Cq), 146.3 (Cq), 150.6 (CH), 160.6 (Cq), 160.9 (Cq), 161.4 (CH), 192.9 (CH). C₂₈H₁₃N₃O₂, calculated m/z 328.1082 (M+1). found m/z 328.1080 (M+1).

### 2-(4-Hydroxyphenyl)-7-(3-hydroxymethyl phenyl)-pyrido[3,2-d]pyrimidine (45)

The product 45 is synthesized from 17 by following the general procedure B for 10 mins and then isolated after purification on a chromatographic silica gel column (CH₂Cl₂/Methanol 98/02) as a yellowish solid with a yield of 88%. MP: 232° C.; IR (ATR, Diamond, cm⁻¹) ν 3393, 3028, 1589, 1454, 1398, 1226, 1004, 898, 804, 741, 698; ¹H NMR (400 MHz, DMSO-d₆) δ: 4.64 (s, 2H, CH₂), 5.33 (s, 1H, OH), 6.94 (d, 2H, J=8.0 Hz, H_{Arom}), 7.49-7.57 (m, 2H, H_{Arom}), 7.86 (d, 1H, J=8.0 Hz, H_{Arom}), 7.92 (s, 1H, H_{Arom}), 8.43 (d, 2H, J=8.0 Hz, H_{Arom}), 8.56 (s, 1H, H₈), 9.36 (s, 1H, H₆), 9.66 (s, 1H, H₄), 10.13 (s, 1H, OH); ¹³C NMR (100 MHz, DMSO-d₆) δ: 62.7 (CH₂), 115.6 (2CH), 125.7 (CH), 126.0 (CH), 127.4 (CH), 127.7 (Cq), 129.1 (CH), 130.3 (2CH), 131.7 (CH), 135.4 (Cq), 137.4 (Cq), 140.5 (Cq), 143.8 (Cq), 146.4 (Cq), 150.7 (CH), 160.6 (Cq), 160.8 (Cq), 161.2 (CH). C₂₀H₁₅N₃O₂, calculated m/z 330.1236 (M+1). found 330.1237 (M+1).

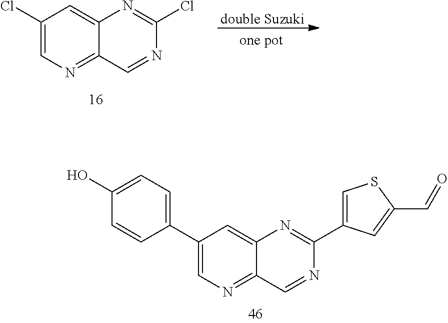 [SMILES: Clc1cnc2cnc(Cl)nc2c1 ; "double Suzuki" ; O=Cc1cc(-c2ncc3ncc(-c4ccc(O)cc4)cc3n2)cs1]

### 2-(3-(5-formyl)thiophyl)-7-(4-Hydroxyphenyl)-pyrido[3,2-d]pyrimidine (46)

Under an argon atmosphere, some 2,7-dichloro-pyrido[3,2-d]pyrimidine 16 (200 mg, 0.99 mmol, 1.0 equiv.) is dissolved in anhydrous toluene and ethanol for analysis (2/1), and then 5-formyl-3-thiophene boronic acid (154.41 mg, 0.99 mmol, 1.0 equiv.), sodium carbonate (209.86 mg, 1.98 mmol, 2.0 equiv.) and tetrakis(triphenylphosphino)palladium(0) (57.77 mg, 0.05 mmol, 0.05 equiv.) are added. After 6 hours of heating to 100° C., the mixture is left with stirring until it returns to room temperature, and then 4-hydroxyphenyl boronic acid (163.86 mg, 1.18 mmol, 1.2 equiv.) and tetrakis(triphenylphosphino)palladium(0) (1.15 g, 0.05 mmol, 0.01 equiv.) are added. The mixture is purged with argon for 5 minutes, and then heating is again set to 100° C. for 1 h. The solvents are evaporated and the thereby obtained residue is purified on a chromatographic silica gel column (DCM/Et₃N 99/1, DCM/MeOH 98/02). The product 46 is obtained as a brown solid, with a yield of 24%. MP>268° C.; IR (ATR, Diamond, cm⁻¹) ν 3033, 1656, 1584, 1519, 1380, 1228, 1174, 950, 834, 725, 665; ¹H NMR (400 MHz, DMSO-d₆) δ: 6.95 (d, 2H, J=12.0 Hz, HArom), 7.87 (d, 2H, J=8.0 Hz, HArom), 8.49 (s, 1H, Hthiop), 8.77 (s, 1H, Hthiop), 9.00 (s, 1H, H8), 9.41 (s, 1H, H6), 9.65 (s, 1H, H4), 9.97 (s, 1H, OH), 10.07 (s, 1H, CHO); ¹³C NMR DEPT (100 MHz, DMSO-d₆) δ: 116.9 (2CH), 129.6 (2CH), 130.3 (CH), 137.0 (CH), 137.8 (CH), 151.9 (CH), 161.8 (CH), 184.8 (CH). C₁₈H₁₂N₃O₂S, calculated m/z 334.0643 (M+1). found 334.0644 (M+1).

### 1.6. Pyrido[3,2-d]pyrimidines with aryls in the position C-2 and amino (het)aryls in position C-7

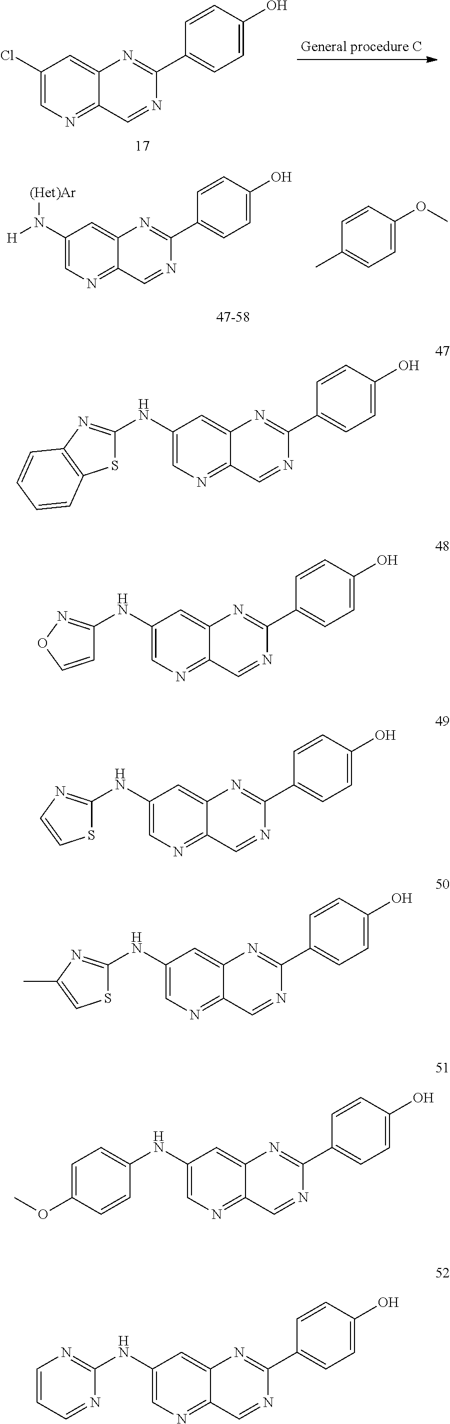

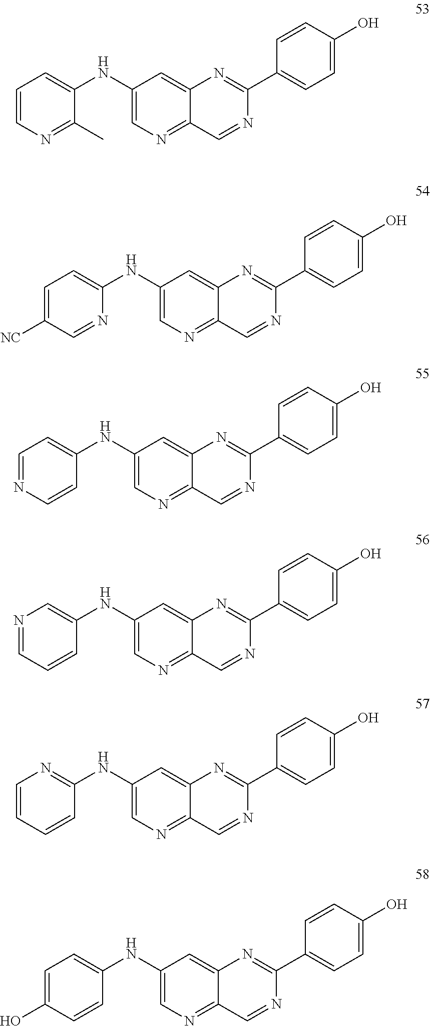 [SMILES: Cc1ncccc1Nc1cnc2cnc(-c3ccc(O)cc3)nc2c1 ; N#Cc1ccc(Nc2cnc3cnc(-c4ccc(O)cc4)nc3c2)nc1 ; Oc1ccc(-c2ncc3ncc(Nc4ccncc4)cc3n2)cc1 ; Oc1ccc(-c2ncc3ncc(Nc4cccnc4)cc3n2)cc1 ; Oc1ccc(-c2ncc3ncc(Nc4ccccn4)cc3n2)cc1 ; Oc1ccc(Nc2cnc3cnc(-c4ccc(O)cc4)nc3c2)cc1]

General Procedure C:

Under an argon atmosphere, in a 5 mL vial, 0.38 mmol (1 equiv.) of 7-chloro-pyrido[3,2-c]pyrimidine are dissolved in 2.5 mL of dioxane. With stirring, are successively added 0.47 mmol (1.2 equiv.) of corresponding amine as well as 0.76 mmol (2.0 equiv.) of potassium carbonate, 0.03 mmol (0.1 equiv.) of palladium acetate and 0.07 mmol (0.2 equiv.) of Xantphos. The reaction mixture is brought to 140° C. with microwave irradiations for the suitable period of time. The solvent is then evaporated and the final compound is obtained by purification on a chromatographic silica gel column or by recrystallization.

### 2-(4-Hydroxyphenyl)-7-(2-benzothiazolamino)-pyrido[3,2-d]pyrimidine (47)

The product 47 is synthesized from 17 by following the general procedure C for 70 mins and then isolated after purification on a chromatographic silica gel column (CH₂Cl₂/NH₃ 99/1, CH₂Cl₂/MeOH, 98/2) as a green solid with a yield of 87%. MP: >268° C.; IR (ATR, Diamond, cm⁻¹) ν 3274, 2987, 1522, 1443, 1221.7, 1159, 751; ¹H NMR (400 MHz, DMSO-d₆) δ: 6.91 (d, 2H, J=8.7 Hz, H_{3′}), 7.26 (t, 1H, J=7.5 Hz, H_{Benzot}), 7.42 (t, 1H, J=7.5 Hz, H_{Benzot}), 7.81 (d, 1H, J=7.5 Hz, H_{Benzot}), 7.89 (d, 1H, J=7.5 Hz, H_{Benzot}), 8.40 (d, 2H, J=8.6 Hz, H₂), 8.87 (d, 1H, J=2.3 Hz, H₈), 9.07 (d, 1H, J=1.7 Hz, H₆), 9.40 (s, 1H, H₄), 10.05 (s, 1H, OH), 11.46 (s, 1H, NH); ¹³C NMR (100 MHz, DMSO-d₆) δ: 115.4 (2CH), 116.4 (CH), 120.1 (CH), 121.3 (CH), 123.3 (CH), 126.1 (CH), 128.0 (Cq), 130.2 (2CH), 130.3 (Cq), 133.7 (Cq), 140.5 (Cq), 144.8 (CH), 147.8 (Cq), 151.4 (Cq), 159.6 (CH), 160.3 (Cq), 160.6 (Cq), 160.9 (Cq). C₂₀H₁₃N₆OS, calculated m/z 372.0919 (M+1). found m/z 372.0907 (M+1).

### 2-(4-Hydroxyphenyl)-7-(3-isoxazolamino)-pyrido[3,2-d]pyrimidine (48)

The product 48 is synthesized from 17 by following the general procedure C for 70 mins and then isolated after purification on a chromatographic silica gel column (CH₂Cl₂/NH₃ 99/1, CH₂Cl₂/MeOH, 98/2) as a yellow solid with a yield of 62%. MP: >268° C.; IR (ATR, Diamond, cm⁻¹) 3319., 3086, 1560, 1459, 1378, 1159, 740; ¹H NMR (400 MHz, DMSO-d₆) δ: 6.43 (s, 1H, H_{Isox4}), 6.89 (d, 2H, J=8.5 Hz, H_{3′}), 8.39 (m, 3H, H_{Arom}, H_{2′}, H_{Isox5}), 8.80 (s, 1H, H₈), 8.83 (d, 1H, J=2.2 Hz, H₆), 9.39 (s, 1H, H₄), 10.01 (s, 1H, OH), 10.41 (s, 1H, NH); ¹³C NMR (100 MHz, DMSO-d₆) δ: 98.3 (CH), 114.3 (CH), 115.4 (2CH), 128.0 (Cq), 130.1 (2CH), 132.9 (Cq), 141.3 (Cq), 144.8 (CH), 148.0 (Cq), 159.2 (CH), 159.3 (Cq), 159.4 (CH), 160.2 (Cq), 160.8 (Cq). C₁₆H₁N₆O₂, calculated m/z 306.0991 (M+1). found m/z 306.0982 (M+1).

### 2-(4-Hydroxyphenyl)-7-(2-thiazolamino)-pyrido[3,2-d]pyrimidine (49)

The product 49 is synthesized from 17 by following the general procedure C for 70 mins and then isolated after purification on a chromatographic silica gel column (CH₂Cl₂/NH₃ 99/1, CH₂Cl₂/MeOH, 98/2) as a yellow solid with a yield of 84%. MP: >268° C. C; IR (ATR, Diamond, cm⁻¹) ν 3280, 2922, 1566, 1443, 1372, 1247, 1155, 840, 697; ¹H NMR (400 MHz, DMSO-d₆) δ: 6.90 (d, 2H, J=8.7 Hz, H_{3′}), 7.17 (d, 1H, J=3.6 Hz, H_{Thiazol}), 7.49 (d, 1H, J=3.6 Hz, H_{Thiazol}), 8.38 (d, 2H, J=8.6 Hz, H_{2′}), 8.84 (d, 1H, J=2.4 Hz, H₈), 8.91 (d, 1H, J=1.7 Hz, H₆), 9.73 (s, 1H, H₄), 10.04 (s, 1H, OH), 11.35 (s, 1H, NH); ¹³C NMR (400 MHz, DMSO-d₆) δ: 111.3 (CH), 114.5 (CH), 115.4 (2CH), 128.1 (Cq), 130.11 (2CH), 133.3 (Cq), 139.14 (CH), 141.04 (Cq), 144.71 (CH), 148.07 (Cq), 159.33 (CH), 160.23 (Cq), 160.84 (Cq), 162.27 (Cq), C₁₆H₁₁N₆OS, calculated m/z 322.0763 (M+1). found m/z 322.0773 (M+1).

### 2-(4-Hydroxyphenyl)-7-(2-(4-methyl)thiazolamino)-pyrido[3,2-d]pyrimidine (50)

The product 50 is synthesized from 17 by following the general procedure C for 70 mins and then isolated after purification on a chromatographic silica gel column (CH₂Cl₂/NH₃ 99/1, CH₂Cl₂/MeOH, 98/2) as a yellow solid with a yield of 95%. MP: >268° C.; IR (ATR, Diamond, cm⁻¹) ν 3274, 3078, 1568, 1384, 1281, 116, 806, 700; ¹H NMR (400 MHz, DMSO-d₆) δ: 2.36 (s, 3H, CH₃), 6.73 (s, 1H, H_{Thiazol}), 6.90 (d, 2H, J=8.7 Hz, H_{3′}), 8.38 (d, 2H, J=8.6 Hz, H₂), 8.80 (d, 1H, J=1.8 Hz, H₈), 8.86 (d, 1H, J=2.0 Hz, H₆), 9.37 (s, 1H, H₄), 10.01 (s, 1H, OH), 11.16 (s, 1H, NH); ¹³C NMR (100 MHz, DMSO-d₆) δ: 17.9 (CH₃), 105.7 (CH), 114.8 (CH), 115.9 (2CH), 128.6 (Cq), 130.6 (2CH), 133.8 (Cq), 141.5 (Cq), 145.2 (CH), 148.6 (Cq), 149.0 (Cq), 159.8 (CH), 160.7 (Cq), 161.4 (Cq), 161.8 (Cq). C₁₇H₁₃N₆OS, calculated m/z 336.0932 (M+1). found m/z 336.0924 (M+1).

### 2-(4-Hydroxyphenyl)-7-(4-methoxyphenylamino)-pyrido[3,2-d]pyrimidine (51)

The product 51 is synthesized from 17 by following the general procedure C for 70 mins and then isolated after purification on a chromatographic silica gel column (CH₂Cl₂/NH₃ 99/1, CH₂Cl₂/MeOH, 98/2) as a red solid with a yield of 57%. MP: 134-136° C.; IR (ATR, Diamond, cm⁻¹) ν 3298, 2991, 1573, 1449, 1373, 1233, 1159, 830; ¹H NMR (400 MHz, DMSO-d₆) δ: 3.78 (s, 3H, CH₃), 6.86 (d, 2H, J=8.2 Hz, H_{3′}), 7.01 (d, 2H, J=8.3 Hz, H_{2″}), 7.22 (s, 1H, H₈), 7.28 (d, 2H, J=8.4 Hz, H_{3″}), 8.32 (d, 2H, J=8.2 Hz, H₂), 8.64 (s, 1H, H₆), 9.18 (s, 1H, H₄), 9.21 (s, 1H, OH), 9.97 (s, 1H, NH); ¹³C NMR (100 MHz, DMSO-d₆) δ: 55.2 (CH₃), 106.5 (CH), 114.8 (2CH), 115.3 (2CH), 123.2 (2CH), 128.3 (Cq), 129.9 (2CH), 132.1 (Cq), 132.3 (Cq), 145.3 (CH), 146.0 (Cq), 148.5 (Cq), 156.0 (Cq), 158.5 (CH), 160.0 (Cq), 160.6 (Cq). C₂₀H₁₆N₄O₂, calculated m/z 345.1352 (M+1). found m/z 345.1360 (M+1).

### 2-(4-Hydroxyphenyl)-7-(2-pyrimidinylamino)-pyrido[3,2-d]pyrimidine (52)

The product 52 is synthesized from 17 by following the general procedure C for 70 mins and then isolated after purification on a chromatographic silica gel column (CH₂Cl₂/NH₃ 99/1, CH₂Cl₂/MeOH, 98/2) as a red solid with a yield of 81%. MP: >268° C.; IR (ATR, Diamond, cm⁻¹) ν 3305, 3109, 1574, 1376, 1157, 803, 701; ¹H NMR (400 MHz, DMSO-d₆) δ: 6.90 (d, 2H, J=8.5 Hz, H_{3′}), 7.08 (t, 1H, J=4.6 Hz, H_{Pyrim5}), 8.38 (d, 2H, J=8.3 Hz, H_{2′}), 8.68 (d, 2H, J=4.6 Hz, H_{Pyrim4} and H_{Pyrim6}), 8.96 (s, 1H, H₈), 9.09 (s, 1H, H₆), 9.38 (s, 1H, H₄), 10.01 (s, 1H, OH), 10.64 (s, 1H, NH); ¹³C NMR (100 MHz, DMSO-d₆) δ: 114.4 (CH), 115.4 (2CH), 116.5 (CH), 128.1 (Cq), 130.0 (2CH), 133.4 (Cq), 140.9 (Cq), 145.9 (CH), 147.7 (Cq), 158.2 (2CH), 159.4 (Cq), 159.4 (CH), 160.1 (Cq), 160.7 (Cq). C₁₇H₁₂N₆O, calculated m/z 317.1151 (M+1). found m/z 317.1142 (M+1).

### 2-(4-Hydroxyphenyl)-7-(3-(2-methyl)-pyridinylamino)-pyrido[3,2-d]pyrimidine (53)

The product 53 is synthesized from 17 by following the general procedure C for 70 mins and then isolated after purification on a chromatographic silica gel column (CH₂Cl₂/NH₃ 99/1, CH₂Cl₂/MeOH, 98/2) as a brown solid with a yield of 97%. MP: 186-188° C.; IR (ATR, Diamond, cm⁻¹) ν 3404, 2974, 1577, 1459, 1376, 1247, 1156, 786; ¹H NMR (400 MHz, DMSO-d₆) δ: 2.38 (s, 3H, CH₃), 6.90 (d, 2H, J=8.7 Hz, H_{3′}), 6.95 (dd, 1H, J=4.9 Hz, J=7.2 Hz, H_{Pyr5}), 7.59 (d, 1H, J=7.0 Hz, H_{Pyr4}), 8.23 (d, 1H, J=3.8 Hz, H_{Pyr6}), 8.38 (d, 2H, J=8.6 Hz, H_{2′}), 8.82 (s, 1H, H₈), 8.85 (d, 1H, J=1.9 Hz, H₆), 9.16 (d, 1H, J=2.3 Hz, H₄), 9.34 (s, 1H, OH), 10.00 (s, 1H, NH); ¹³C NMR (100 MHz, DMSO-d₆) δ: 17.1 (CH₃), 115.4 (2CH), 115.5 (CH), 117.3 (CH), 120.9 (Cq), 128.2 (Cq), 130.0 (2CH), 133.1 (Cq), 138.7 (CH), 142.2 (Cq), 144.5 (CH), 146.9 (CH), 147.9 (Cq), 152.5 (Cq), 195.1 (CH), 160.1 (Cq), 160.6 (Cq). C₁₉H₁₅N₆O, calculated m/z 330.1355 (M+1). found m/z 330.1347 (M+1).

### 2-(4-Hydroxyphenyl)-7-(2-(5-cyano)pyridinylamino)-pyrido[3,2-d]pyrimidine (54)

The product 54 is synthesized from 17 by following the general procedure C for 70 mins and then isolated after purification on a chromatographic silica gel column (CH₂Cl₂/MeOH, 95/5) as a red solid with a yield of 80%. MP: >268° C.; IR (ATR, Diamond, cm⁻¹) ν 3311, 3113, 2224, 1573, 1433, 1374, 1153, 828; ¹H NMR (400 MHz, DMSO-d₆) δ: 6.89 (d, 2H, J=8.6 Hz, H_{3′}), 7.07 (d, 1H, J=8.7 Hz, H_{Pyr5}), 8.03 (dd, 1H, J=2.1 Hz, J=8.7 Hz, H_{Pyr6}), 8.35 (d, 2H, J=8.6 Hz, H_{2′}), 8.77 (d, 1H, J=1.8 Hz, H_{Pyr3}), 8.86 (d, 1H, J=2.1 Hz, H₈), 8.92 (d, 1H, J=1.5 Hz, H₆), 9.34 (s, 1H, H₄), 10.01 (s, 1H, OH), 10.59 (s, 1H, NH); ¹³C NMR (100 MHz, DMSO-d₆) δ: 100.0 (Cq), 112.3 (CH), 115.4 (2CH), 117.2 (CH), 117.8 (Cq), 128.0 (Cq), 130.1 (2CH), 133.6 (Cq), 140.0 (CH), 140.3 (Cq), 145.7 (CH), 147.5 (Cq), 152.2 (CH), 156.6 (Cq), 159.4 (CH), 160.2 (Cq), 160.8 (Cq). C₁₉H₁₂N₆O, calculated m/z 341.1151 (M+1). found m/z 341.1154 (M+1).

### 2-(4-Hydroxyphenyl)-7-(4-pyridinylamino)-pyrido[3,2-d]pyrimidine (55)

The product 55 is synthesized from 17 by following the general procedure C for 70 mins and then isolated after purification after recrystallization from methanol as a brown solid with a yield of 72%. MP: >268° C.; IR (ATR, Diamond, cm⁻¹) ν 3014, 1572, 1453, 1290, 1165, 810; ¹H NMR (400 MHz, DMSO-d₆) δ: 6.89 (d, 2H, J=8.7 Hz, H_{3′}), 7.29 (d, 2H, J=6.2 Hz, H_{Pyr3}), 7.86 (d, 1H, J=2.3 Hz, H₈), 7.94 (d, 1H, J=4.9 Hz, H₆), 8.38 (d, 2H, J=8.7 Hz, H_{2′}), 8.43 (d, 2H, J=6.1 Hz, H_{Pyr2}), 8.80 (d, 1H, J=2.5 Hz, H₄), 9.39 (s, 1H, OH), 9.84 (broad, 1H, NH); ¹³C NMR (100 MHz, DMSO-d₆) δ: 111.8 (2CH), 113.6 (CH), 115.5 (2CH), 127.7 (Cq), 130.1 (2CH), 133.4 (Cq), 141.9 (Cq), 146.2 (CH), 147.4 (Cq), 147.8 (Cq), 149.3 (Cq), 150.6 (2CH), 159.4 (CH), 160.9 (Cq). C₁₈H₁₃N₅O, calculated m/z 316.1198 (M+1). found m/z 316.1199 (M+1).

### 2-(4-Hydroxyphenyl)-7-(3-pyridinylamino)-pyrido[3,2-d]pyrimidine (56)

The product 56 is synthesized from 17 by following the general procedure C for 70 mins and then isolated after purification on a chromatographic silica gel column (CH₂Cl₂/NH₃ 99/1 and then CH₂Cl₂/MeOH, 98/2) as a yellow solid with a yield of 71%. MP: >268° C.; IR (ATR, Diamond, cm⁻¹) ν 3256, 2913, 1556, 1451, 1243, 1158, 696; ¹H NMR (400 MHz, DMSO-d₆) δ: 6.87 (d, 2H, J=8.6 Hz, H_{3′}), 7.42 (dd, 1H, J=4.6 Hz, J=8.1 Hz, H_{Pyr5}), 7.51 (s, 1H, H_{Pyr6}), 7.84 (d, 1H, J=8.2 Hz, H_{Pyr4}), 8.34 (m, 3H, H_{Arom}, H_{2′} and H_{Pyr2}), 8.59 (s, 1H, H₈), 8.73 (d, 1H, J=2.1 Hz, H₆), 9.29 (s, 1H, H₄), 9.55 (s, 1H, OH), 10.00 (s, 1H, NH); ¹³C NMR (100 MHz, DMSO-d₆) δ: 109.1 (CH), 115.3 (2CH), 124.1 (CH), 126.6 (CH), 128.1 (Cq), 130.0 (2CH), 132.7 (CH), 136.8 (Cq), 142.1 (CH), 144.0 (CH), 144.1 (Cq), 145.5 (CH), 148.1 (Cq), 158.9 (CH), 160.1 (Cq), 160.7 (Cq); HRMS (EI-MS): C₁₈H₁₃N₅O, calculated m/z 316.1198 (M+1). found m/z 316.1189 (M+1).

### 2-(4-Hydroxyphenyl)-7-(2-pyridinylamino)-pyrido[3,2-d]pyrimidine (57). The product 57 is synthesized from 17 by following the general procedure C for 70 mins and then isolated after purification on a chromatographic silica gel column (CH₂Cl₂/NH₃ 99/1 puis CH₂Cl₂/MeOH, 98/2) as a red solid with a yield of 90%. MP: >268° C.; IR (ATR, Diamond, cm⁻¹) ν 3352, 3011, 1576, 1382, 1280, 1166, 775; ¹H NMR (400 MHz, DMSO-d₆) δ: 6.90 (d, 2H, J=8.7 Hz, H_{3′}), 6.96 (dd, 1H, J=5.3 Hz, J=6.5 Hz, H_{Pyr5}), 7.05 (d, 1H, J=8.3 Hz, H_{Pyr4}), 7.71 (m, 1H, H_{Pyr6}), 8.38 (3H, H_{Arom}, H_{2′} and H_{Pyr3}), 8.90 (d, 1H, J=2.3 Hz, H₈), 9.04 (d, 1H, J=1.7 Hz, H₆), 9.33 (s, 1H, H₄), 9.99 (s, 1H, OH), 10.13 (s, 1H, NH); ¹³C NMR (100 MHz, DMSO-d₆) δ: 112.4 (CH), 114.5 (CH), 115.3 (2CH), 116.6 (CH), 128.2 (Cq), 130.0 (CH), 132.9 (Cq), 137.8 (CH), 141.8 (Cq), 145.8 (CH), 147.3 (CH), 148.1 (Cq), 154.6 (Cq), 159.1 (CH), 160.1 (Cq), 160.7 (Cq). C₁₈H₁₃N₅O, calculated m/z 316.1198 (M+1). found m/z 316.1210 (M+1).

### 2-(4-Hydroxyphenyl)-7-(4-hydroxyphenylamino)-pyrido[3,2-d]pyrimidine (58)

The product 58 is synthesized from 17 by following the general procedure C for 70 mins and then isolated after purification on a chromatographic silica gel column (CH₂Cl₂/NH₃ 99/1 puis CH₂Cl₂/MeOH, 98/2) as an offset white solid with a yield of 80%. MP: 168-170° C.; IR (ATR, Diamond, cm⁻¹) ν 3386, 2234, 1604, 1548, 1461, 1399, 1236, 1159, 959, 837; ¹H NMR (400 MHz, DMSO-d₆) δ: 6.85-6.89 (m, 4H, H_{Arom}), 7.15-7.19 (m, 3H, H_{Arom}), 8.32 (d, 2H, J=12.0 Hz, H_{Arom}+H₈), 8.62 (d, 1H, J=4.0 Hz, H₆), 9.07 (s, 1H, H₄), 9.20 (s, 1H, OH), 9.45 (s, 1H, OH), 9.95 (s, 1H, NH); ¹³C NMR (400 MHz, DMSO-d₆) δ: 106.2 (CH), 115.3 (2CH), 116.1 (2CH), 123.9 (2CH), 128.4 (Cq), 130.0 (2CH), 130.7 (Cq), 132.0 (Cq), 145.3 (CH), 146.5 (Cq), 148.7 (Cq), 154.4 (Cq), 158.5 (CH), 160.0 (Cq), 160.7 (Cq). C₁₃H₁₄N₄O₂, calculated m/z 331.1195 (M+1). found m/z 331.1183 (M+1).

### 1.7. Compounds with Amino-Aryls in Position C-2 and Aryls in Position C-7

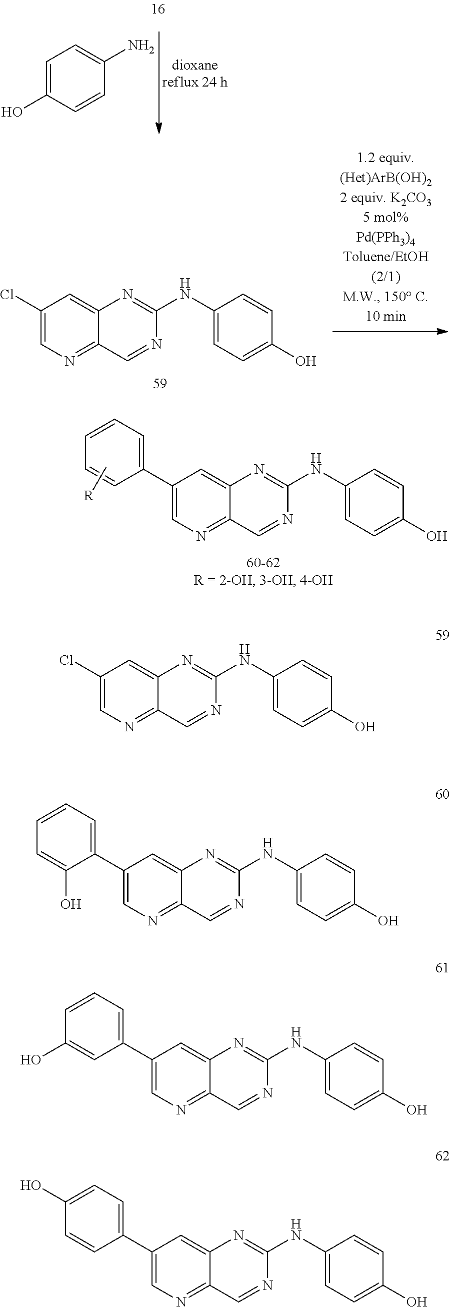

### 7-Chloro-2-(4-hydroxyphenylamino)-pyrido[3,2-d]pyrimidine (59)

In a 50 mL flask, are introduced 200 mg (0.99 mmol, 1 equiv.) of 2,7-dichloropyrido[3,2-d]pyrimidine 16, as well as 131 mg (1.19 mmol, 1.2 equiv.) of 4-hydroxyaniline and 7 mL of dioxane. Next, the reaction mixture is refluxed for 24 hours. The solvant is evaporated and the obtained residue is then purified by flash chromatography on silica gel (CH₂Cl₂/NH₃ 99/1, CH₂Cl₂/MeOH, 98/2) in order to obtain the compound 59 as an orange solid with a yield of 60%. MP: 232-234° C.; IR (ATR, Diamond, cm⁻¹) ν 3274, 1607, 1445, 1338, 1198, 1072, 820, 714; ¹H NMR (400 MHz, DMSO-d₆) δ: 6.72 (d, 2H, J=8.7 Hz, H_{3′}), 7.67 (d, 2H, J=8.3 Hz, H_{2′}), 8.13 (d, 1H, J=1.2 Hz, H₈), 8.63 (d, 1H, J=1.9 Hz, H₆), 9.17 (s, 1H, H₄), 9.26 (s, 1H, NH), 9.96 (s, 1H, OH); ¹³C NMR (100 MHz, DMSO-d₆) δ: 114.9 (2CH), 121.2 (CH), 131.2 (Cq), 131.5 (2CH), 134.7 (Cq), 134.9 (Cq), 145.6 (CH), 147.5 (Cq), 152.9 (Cq), 157.3 (Cq), 162.4 (CH). C₁₃H₉ClN₄O, calculated m/z 273.0543 (M+1). found m/z 273.0539 (M+1).

### 2-(4-Hydroxyphenylamino)-7-(2-hydroxyphenyl)-pyrido[3,2-d]pyrimidine (60)

The product 60 is synthesized from 59 by following the general procedure B for 10 mins and then isolated after purification on a chromatographic silica gel column (CH₂Cl₂/NH₃ 99/1, CH₂Cl₂/MeOH 98/2) as an orange solid with a yield of 66%. MP: 262-264° C.; IR (ATR, Diamond, cm⁻¹) ν 3233, 3032, 1602, 1546, 1434, 1366, 1213, 826, 725; ¹H NMR (400 MHz, DMSO-d₆) δ: 6.73 (d, 2H, J=8.7 Hz, H_{3′}), 6.94-7.04 (m, 2H, H_{4″} and H_{6″}), 7.29 (t, 1H, J=7.4 Hz, H_{5″}), 7.48 (d, 1H, J=7.0 Hz, H_{3″}), 7.71 (d, 2H, J=8.5 Hz, H_{2′}), 8.05 (s, 1H, H₈), 8.87 (d, 1H, J=1.3 Hz, H₆), 9.11 (s, 1H, H₄), 9.24 (s, 1H, NH), 9.77 (s, 1H, OH), 10.00 (s, 1H, OH); ¹³C NMR (100 MHz, DMSO-d₆) δ: 114.9 (2CH), 116.2 (CH), 119.7 (CH), 120.9 (CH), 123.4 (Cq), 130.2 (CH), 130.6 (CH), 131.7 (2CH), 131.7 (Cq), 134.7 (Cq), 139.1 (Cq), 147.1 (Cq), 148.1 (CH), 152.6 (Cq), 154.7 (Cq), 157.1 (Cq), 162.0 (CH). C₁₃H₁₄N₄O₂, calculated m/z 331.1195 (M+1). found m/z 331.1211 (M+1).

### 2-(4-Hydroxyphenylamino)-7-(3-hydroxyphenyl)-pyrido[3,2-d]pyrimidine (61)

The product 61 is synthesized from 59 by following the general procedure B for 10 mins and then isolated after purification on a chromatographic silica gel column (CH₂Cl₂/NH₃ 99/1, CH₂Cl₂/MeOH 98/2) as an orange solid with a yield of 60%. MP: >268° C.; IR (ATR, Diamond, cm⁻¹) ν 3276, 2923, 2601, 1595, 1508, 1399, 1219, 794; ¹H NMR (400 MHz, DMSO-d₆) δ: 6.73 (d, 2H, J=8.8 Hz, H_{3′}), 6.87-6.91 (m, 1H, H_{6″}), 7.23 (s, 1H, H_{2″}), 7.30-7.35 (m, 2H, H_{4″} and H_{5″}), 7.72 (d, 2H, J=8.6 Hz, H_{2′}), 8.08 (d, 1H, J=1.0 Hz, H₈), 8.93 (d, 1H, J=1.8 Hz, H₆), 9.11 (s, 1H, H₄), 9.25 (s, 1H, NH), 9.70 (s, 1H, OH), 9.82 (s, 1H, OH); ¹³C NMR (100 MHz, DMSO-d₆) δ: 114.1 (CH), 114.9 (2CH), 116.0 (CH), 118.2 (CH), 120.9 (CH), 129.4 (CH), 130.3 (2CH), 131.7 (Cq), 135.5 (Cq), 137.4 (Cq), 140.1 (Cq), 146.0 (CH), 147.2 (Cq), 152.6 (Cq), 157.2 (Cq), 158.0 (Cq), 162.2 (CH). C₁₉H₁₄N₄O₂, calculated m/z 331.1195 (M+1). found m/z 331.1200 (M+1).

### 2-(4-Hydroxyphenylamino)-7-(4-hydroxyphenyl)-pyrido[3,2-d]pyrimidine (62)

The product 62 is synthesized from 59 by following the general procedure B for 10 mins and then isolated after purification on a chromatographic silica gel column (CH₂Cl₂/NH₃ 99/1, CH₂Cl₂/MeOH 98/2) as an orange solid with a yield of 80%. MP: >268° C.; IR (ATR, Diamond, cm⁻¹) ν 3253, 2923, 1599, 1512, 1359, 1210, 1171, 819; ¹H NMR (400 MHz, DMSO-d₆) δ: 6.74 (d, 2H, J=8.6 Hz, H_{3″}), 6.91 (d, 2H, J=8.3, H_{3′}) 7.69-7.80 (m, 4H, H_{Arom}, H_{2′} and H_{2″}), 8.06 (s, 1H, H₈), 8.97 (s, 1H, H₆), 9.11 (s, 1H, H₄), 9.21 (s, 1H, NH), 9.76 (s, 1H, OH), 9.87 (s, 1H, OH); ¹³C NMR (100 MHz, DMSO-d₆) δ: 114.9 (2CH), 116.0 (2CH), 120.9 (2CH), 126.5 (Cq), 127.8 (CH), 128.8 (2CH), 131.7 (Cq), 134.9 (Cq), 140.0 (Cq), 145.9 (CH), 147.4 (Cq), 152.6 (Cq), 157.2 (Cq), 158.5 (Cq), 161.9 (CH). C₁₉H₁₄N₄O₂, calculated m/z 331.1195 (M+1). found m/z 331.1190 (M+1).

### 1.8. Amidification in Position C-7 from 17

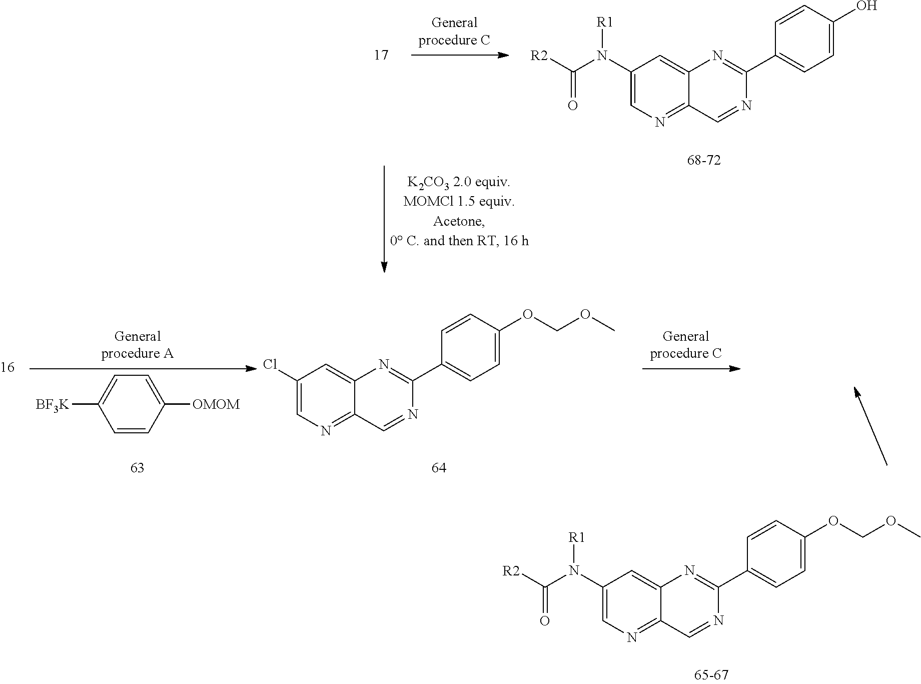

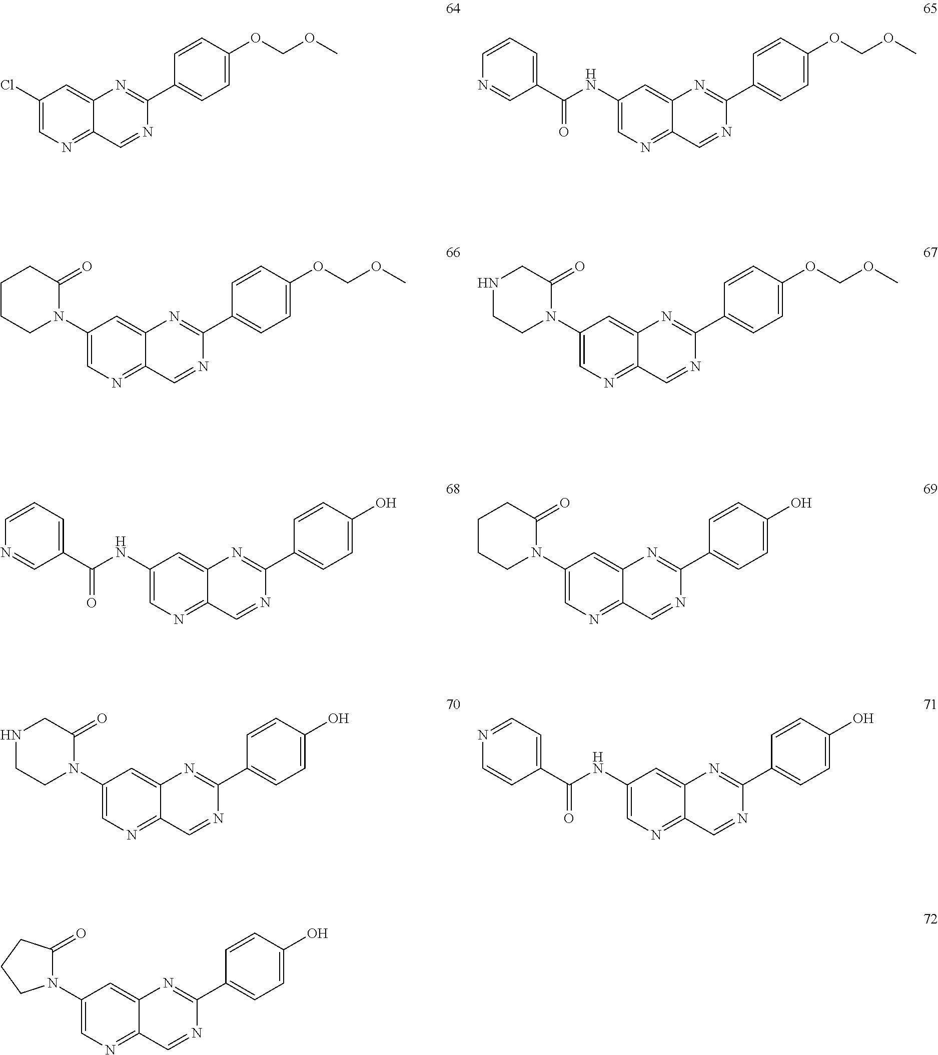

### 7-Chloro-2-(4-methoxymethoxy-phenyl)-pyrido[3,2-d]pyrimidine (64)

The product 64 may be obtained from 16 according to the general procedure A by using 1.1 equiv. of 63 or from 17 according to the following procedure: in a flask, a mixture consisting of 343 mg (1.33 mmol, 1.0 equiv.) of 17, 368 mg (1.66 mmol, 2.0 equiv.) of K₂CO₃ and 15 mL of acetone are vigorously stirred at 0° C. Next 152 μL (1.99 mmol, 1.5 equiv.) of chloromethylmethyl ether are added dropwise. The whole is left with stirring at room temperature for 16 h. After evaporation of the solvant, the thereby obtained residue is taken up with a minimum of water (2 mL) and the aqueous phase is then extracted with dichloromethane (2×10 mL). The organic phase is dried on MgSO₄, filtered and then concentrated under reduced pressure. The compound 64 is isolated after purification by silica gel chromatography (eluent petroleum ether/CH₂Cl₂ 2/8), as a white solid with a yield of 71%. MP: >268° C.; IR (ATR, Diamond, cm⁻¹) ν 3047, 2926, 2824, 1586, 1441, 1231, 1109, 1073, 996, 921, 804, 737, 698; ¹H NMR (400 MHz, DMSO-d₆) 8: ¹H NMR (400 MHz, DMSO-d₆) δ ppm: 3.42 (s, 3H, CH₃), 5.31 (s, 2H, CH₂), 7.21 (d, 2H, J=12.0 Hz, H_{Ar}), 8.50 (d, 2H, J=8.0 Hz, H_{Ar}), 8.64 (s, 1H, H₈), 9.07 (s, 1H, H₆), 9.74 (s, 1H, H₄). ¹³C NMR (400 MHz, DMSO-d₆) δ ppm: 55.8 (CH₃), 93.7 (CH₂), 116.2 (2CH), 129.9 (Cq), 130.2 (2CH), 134.2 (CH), 135.4 (Cq), 136.8 (Cq), 146.4 (Cq), 151.0 (CH), 159.6 (Cq), 160.8 (Cq), 161.7 (CH).

### N-[2-(4-Methoxymethoxy-phenyl)-pyrido[3,2-d]pyrimidin-7-yl]-nicotinamide (65)

The product 65 is synthesized from 64 by following the general procedure C for 60 mins and then isolated after purification on a chromatographic silica gel column (CH₂Cl₂/NH₃ 99/1, eluent: CH₂Cl₂/MeOH, 95/5) as a pinkish solid with a yield of 80%. MP: 232° C.; IR (ATR, Diamond, cm⁻¹) ν 3335, 2361, 1687, 1559, 1457, 1375, 1247, 1155, 995, 701; ¹H NMR (400 MHz, DMSO-d₆) δ: 3.42 (s, 3H, CH₃), 5.30 (s, 2H, CH₂), 7.18 (d, 2H, J=8.0 Hz, H_{Ar}), 7.62 (m, 1H, H_{Isonic}), 8.37 (d, 1H, J=8.0 Hz, H_{Isonic}), 8.50 (d, 2H, J=8.0 Hz, H_{Ar}), 8.82 (d, 1H, J=8.0 Hz, H_{Isonic}), 8.89 (s, 1H, H₈), 9.21 (s, 1H, H_{Isonic}), 9.22 (d, 1H, J=4.0 Hz, H₆), 9.54 (s, 1H, H₄), 11.21 (s, 1H, NH); ¹³C NMR (100 MHz, DMSO-d₆) δ: 56.2 (CH₃), 94.2 (CH₂), 116.5 (2CH), 121.4 (CH), 124.1 (CH), 130.2 (Cq), 130.4 (2CH), 130.8 (Cq), 135.4 (Cq), 136.2 (CH), 139.9 (Cq), 146.8 (CH), 147.5 (Cq), 149.3 (CH), 153.2 (CH), 159.8 (Cq), 160.8 (CH), 160.9 (Cq), 165.9 (CO). C₂₁H₁₇N₅O₃, calculated m/z 388.1410 (M+1). found m/z 388.1400 (M+1).

### 1-[2-(4-Methoxymethoxy-phenyl)-pyrido[3,2-d]pyrimidin-7-yl]-piperidin-2-one (66)

The product 66 is synthesized from 64 by following the general procedure C for 60 mins and then isolated after purification on a chromatographic silica gel column (CH₂Cl₂/NH₃ 99/1, eluent: CH₂Cl₂/MeOH, 98/2) as a pinkish solid with a yield of 88%. MP: 202-204° C.; IR (ATR, Diamond, cm⁻¹) ν 3335, 2359, 2148, 1647, 1596, 1454, 1325, 1146, 979, 805, 700; ¹H NMR (400 MHz, DMSO-d₆) δ: 1.90 (m, 4H, H_{Piper}), 2.54 (m, 2H, H_{Piper}), 3.42 (s, 3H, CH₃), 3.88 (m, 2H, H_{Piper}), 5.30 (s, 2H, CH₂), 7.20 (d, 2H, J=8.0 Hz, H_{Ar}), 8.26 (d, 1H, J=4.0 Hz, H₈), 8.50 (d, 2H, J=8.0 Hz, H_{Ar}), 9.08 (s, 1H, H₄), 9.61 (s, 1H, H₆); ¹³C NMR (100 MHz, DMSO-d₆) δ: 20.8 (CH₂), 22.7 (CH₂), 32.7 (CH₂), 49.8 (CH₂), 55.7 (CH₃), 93.7 (CH₂), 116.1 (2CH), 127.2 (CH), 129.9 (2CH), 130.3 (Cq), 135.4 Cq), 144.4 (Cq), 146.9 (Cq), 151.2 (CH), 159.3 (Cq), 160.2 (Cq), 160.6 (CH), 170.2 (CO). C₂₀H₂₀N₄O₃Na, calculated m/z 387.1433 (M+1). found m/z 387.1422 (M+1).

### 1-[2-(4-Methoxymethoxy-phenyl)-pyrido[3,2-d]pyrimidin-7-yl]-piperazin-2-one (67)

The product 67 is synthesized from 64 by following the general procedure C for 60 mins and then isolated after purification on a chromatographic silica gel column (CH₂Cl₂/NH₃ 99/1, eluent: CH₂Cl₂/MeOH, 98/2) as a pale yellow solid with a yield of 78%. MP: 200-202° C.; IR (ATR, Diamond, cm⁻¹) ν 3334, 2958, 2148, 1647, 1448, 1256, 1104, 971, 799; ¹H NMR (400 MHz, DMSO-d₆) δ: 3.11 (s, 2H, H_{Piper}), 3.42 (s, 3H, CH₃), 3.53 (s, 2H, H_{Piper}), 3.90 (t, 2H, J=4 Hz, H_{Piper}), 5.31 (s, 2H, CH₂), 7.20 (d, 2H, J=8 Hz, H_{Ar}), 8.33 (s, 1H, H₈), 8.50 (d, 2H, J=8 Hz, H_{Ar}), 9.17 (s, 1H, H₆), 9.63 (s, 1H, H₄); ¹³C NMR (100 MHz, DMSO-d₆) δ: 42.5 (CH₂), 49.9 (CH₂), 50.6 (CH₂), 55.7 (CH₃), 93.7 (CH₂), 116.7 (2CH), 126.8 (CH), 130.0 (2CH), 130.2 (Cq), 135.5 (Cq), 143.5 (Cq), 146.9 (Cq), 150.4 (CH), 159.4 (Cq), 160.3 (Cq), 160.7 (CH), 168.9 (CO). C₁₉H₁₉N₆O₃, calculated m/z 366.1566 (M+1). found m/z 366.1549 (M+1).

### N-[2-(4-Hydroxy-phenyl)-pyrido[3,2-d]pyrimidin-7-yl]nicotinamide (68)

The product 68 is synthesized from 17 by following the general procedure C for 60 mins and then isolated after purification on a chromatographic silica gel column (CH₂Cl₂/NH₃ 99/1, CH₂Cl₂/MeOH, 95/5) as a pinkish solid with a yield of 80%. It may also be obtained from 65 by treatment with aqueous 10% hydrochloric acid in MeOH with quantitative yield. MP: 226-228° C.; IR (ATR, Diamond, cm⁻¹) ν 2361, 1687, 1559, 1457, 1375, 1247, 1155, 995, 701; ¹H NMR (400 MHz, DMSO-d₆) δ: 6.93 (d, 2H, J=8.0 Hz, H_{Arom}), 7.63-7.66 (m, 1H, H_{Arom}), 8.42 (d, 3H, J=8.0 Hz, H_{Arom}), 8.83 (d, 1H, J=4.0 Hz, H_{Arom}), 8.88 (d, 1H, J=4.0 Hz, H₈), 9.23 (m, 1H, H_{Arom}), 9.26 (d, 1H, J=4.0 Hz, H₆), 9.52 (s, 1H, H₄), 10.11 (s, 1H, OH), 11.31 (s, 1H, NH); ¹³C NMR (100 MHz, DMSO-d₆) δ: 115.5 (2CH), 121.0 (CH), 123.6 (CH), 127.8 (Cq), 129.7 (Cq), 130.28 (2CH), 134.9 (Cq), 135.9 (CH), 139.4 (Cq), 146.0 (CH), 147.1 (Cq), 148.9 (CH), 152.6 (CH), 160.2 (CH), 160.5 (Cq), 160.9 (Cq), 165.4 (Cq). C₁₉H₁₃N₅O₂, calculated m/z 344.1147 (M+1). found m/z 344.1146 (M+1).

### 1-[2-(4-Hydroxy-phenyl)-pyrido[3,2-d]pyrimidin-7-yl]-piperidin-2-one (69)

The product 69 is synthesized from 17 by following the general procedure C for 60 mins and then isolated after purification on a chromatographic silica gel column (CH₂Cl₂/MeOH, 98/2) as a yellowish solid with a yield of 79%. It may also be obtained from 66 by treatment with aqueous 10% hydrochloric acid in MeOH with quantitative yield. MP: 268-270° C.; IR (ATR, Diamond, cm⁻¹) ν 2938, 1600, 1491, 1452, 1326, 1264, 1157, 807, 700; ¹H NMR (400 MHz, DMSO-d₆) δ: 1.90-1.96 (m, 4H, H_{Piper}), 2.53-2.55 (m, 2H, H_{Piper}), 3.87-3.90 (m, 2H, H_{Piper}), 6.93 (d, 2H, J=8.0 Hz, H_{Arom}), 8.22 (s, 1H, H₈), 8.40 (d, 2H, J=8.0 Hz, H_{Arom}), 9.04 (d, 2H, J=4.0 Hz, H₆), 9.57 (s, 1H, H₄), 10.08 (s, 1H, OH); ¹³C NMR (100 MHz, DMSO-d₆) δ: 20.8 (CH₂), 22.7 (CH₂), 32.7 (CH₂), 49.8 (CH₂), 115.6 (2CH), 127.3 (CH), 127.7 (Cq), 130.2 (2CH), 135.3 (Cq), 144.3 (Cq), 146.9 (Cq), 150.8 (CH), 160.51 (Cq), 160.56 (Cq), 160.6 (CH), 170.1 (Cq). C₁₈H₁₆N₄O₂Na, calculated m/z 343.1171 (M+1). found m/z 343.1170 (M+1).

### 1-[2-(4-Hydroxy-phenyl)-pyrido[3,2-d]pyrimidin-7-yl]-piperazin-2-one (70)

The product 70 is synthesized from 17 by following the general procedure C for 60 mins and then isolated after purification on a chromatographic silica gel column (CH₂Cl₂/MeOH, 98/2) as a yellowish solid with a yield of 79%. It may also be obtained from 67 by treatment with aqueous 10% hydrochloric acid in MeOH with quantitative yield. MP: >268° C.; IR (ATR, Diamond, cm⁻¹) ν 3295, 2490, 2157, 1639, 1566, 1448, 1371, 1261, 1164, 847; ¹H NMR (400 MHz, DMSO-d₆) δ: 3.11 (s, 2H, H_{Piper}), 3.52 (s, 2H, H_{Piper}), 3.89 (s, 2H, H_{Piper}), 6.93 (d, 2H, J=8.0 Hz, H_{Ar}), 8.28 (s, 1H, H₈), 8.40 (d, 2H, J=8.0 Hz, H_{Ar}), 9.13 (s, 1H, H₆), 9.58 (s, 1H, H₄), 10.12 (s, 1H, OH); ¹³C NMR (100 MHz, DMSO-d₆) δ: 42.5 (CH₂), 50.0 (CH₂), 50.6 (CH₂), 115.64 (2CH), 126.8 (CH), 127.7 (Cq), 130.2 (2CH), 135.4 (Cq), 143.4 (Cq), 146.9 (Cq), 150.1 (CH), 160.5 (Cq), 160.6 (CH), 160.7 (Cq), 168.8 (CO). C₁₇H₁₅N₅O₂, calculated m/z 322.1304 (M+1). found m/z 322.1306 (M+1).

### N-[2-(4-Hydroxy-phenyl)-pyrido[3,2-d]pyrimidin-7-yl]isonicotinamide (71)

The product 71 is synthesized from 17 by following the general procedure C for 60 mins and then isolated after purification on a chromatographic silica gel column (CH₂Cl₂/NH₃ 99/1, CH₂Cl₂/MeOH, 95/5) as a yellowish solid with a yield of 89%. MP: 230-232° C.; IR (ATR, Diamond, cm⁻¹) ν 3214, 2957, 1672, 1553, 1448, 1374, 1230, 1157, 807, 670; ¹H NMR (400 MHz, DMSO-d₆) δ: 6.93 (d, 2H, J=8.0 Hz, H_{Arom}), 7.95 (d, 2H, J=4.0 Hz, H_{Arom}), 8.42 (d, 2H, J=4.0 Hz, H_{Arom}), 8.86 (s, 3H, H_{Arom}+H₈), 9.22 (s, 1H, H₆), 9.54 (s, 1H, H₄), 10.08 (s, 1H, OH), 11.26 (s, 1H, NH); ¹³C NMR (100 MHz, DMSO-d₆) δ: 115.6 (2CH), 121.3 (CH), 121.6 (2CH), 127.6 (CH), 130.3 (2CH), 135.0 (Cq), 139.1 (Cq), 140.9 (Cq), 146.0 (CH), 147.0 (Cq), 150.4 (2CH), 160.3 (CH), 160.5 (Cq), 160.9 (Cq), 165.4 (Cq). C₁₉H₁₃N₅O₂, calculated m/z 344.1147 (M+1). found m/z 344.1146 (M+1).

### 1-[2-(4-Hydroxy-phenyl)-pyrido[3,2-d]pyrimidin-7-yl]-pyrrolidin-2-one (72)

The product 72 is synthesized from 17 by following the general procedure C for 60 mins and then isolated after purification on a chromatographic silica gel column (CH₂Cl₂/MeOH, 99/01) as a pinkish solid with a yield of 93%. MP>268° C.; IR (ATR, Diamond, cm⁻¹) ν 3213, 1665, 1517, 1460, 1343, 1260, 1098, 806, 734; ¹H NMR (400 MHz, DMSO-d₆) δ: 2.11-2.19 (m, 2H, H_{py4}), 2.61 (t, 2H, J=8.0 Hz, H_{py3}), 4.03 (t, 2H, J=8.0 Hz, H_{py5}), 6.92 (d, 2H, J=8.0 Hz, H_{Arom}), 8.30 (s, 1H, H₈), 8.38 (d, 2H, J=8.0 Hz, H_{Arom}), 9.50 (5, 2H, H₆ and H₄); ¹³C NMR (100 MHz, DMSO-d₆) δ: 17.0 (CH₂), 31.61 (CH₂), 47.29 (CH₂), 115.2 (2CH), 120.0 (CH), 127.73 (CH), 129.8 (2CH), 134.7 (Cq), 139.6 (Cq), 144.3 (Cq), 146.4 (Cq), 159.8 (CH), 160.1 (Cq), 160.7 (Cq), 174.8 (CO); C₁₇H₁₄N₄O₂, calculated m/z 307,1190 (M+1). found m/z 307,1189 (M+1).

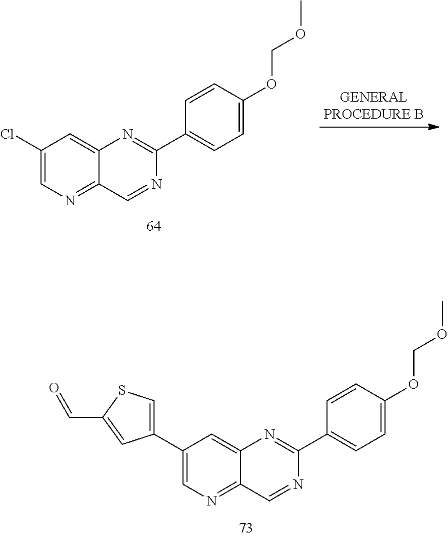 [SMILES: COCOc1ccc(-c2ncc3ncc(Cl)cc3n2)cc1 ; COCOc1ccc(-c2ncc3ncc(-c4csc(C=O)c4)cc3n2)cc1]

### 2-(4-methoxymethoxy-phenyl)-7-(3-(5-formyl)thiophyl)-pyrido[3,2-d]pyrimidine (73)

The product 73 is synthesized from 64 by following the general procedure B for 10 mins and then isolated after purification on a chromatographic silica gel column (CH₂Cl₂/MeOH, 98/2) as a brown solid with a yield of 89%. MP: >268° C.; IR (ATR, Diamond, cm⁻¹) ν 3082, 1678, 1588, 1393, 1237, 1152, 1076, 1003, 916, 850, 790, 701; ¹H NMR (400 MHz, DMSO-d₆) δ: 3.43 (s, 3H, CH₃), 5.31 (s, 2H, CH₂), 7.21 (d, 2H, J=8.0 Hz, H_{Arom}), 8.52 (d, 2H, J=12.0 Hz, H_{Arom}), 8.81 (s, 1H, Hₜₕᵢₒ₅), 8.89 (s, 1H, Hₜₕᵢₒ₂), 9.03 (s, 1H, H₉), 9.54 (s, 1H, H₆), 9.68 (s, 1H, H₄), 10.03 (s, 1H, OH); ¹³C NMR (100 MHz, DMSO-d₆) δ: 55.7 (CH₃), 93.7 (CH₂), 116.1 (2CH), 130.0 (2CH), 130.2 (Cq), 131.0 (CH), 134.26 (CH), 134.29 (Cq), 136.5 (CH), 137.6 (Cq), 138.1 (Cq), 144.5 (Cq), 146.5 (Cq), 150.4 (CH), 159.4 (Cq), 160.5 (Cq), 161.3 (CH), 184.1 (CHO); C₂₀H₁₅N₃O₃S, calculated m/z 378.0908. found 378.0906.

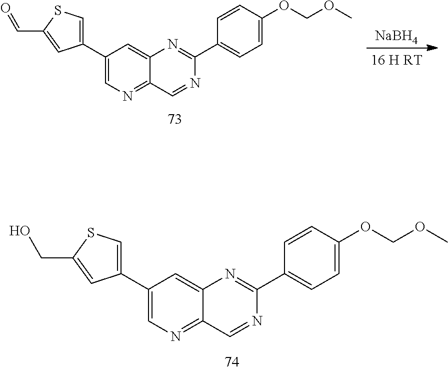

### 2-(4-methoxymethoxy-phenyl)-7-(3-(5-hydroxymethyl)thiophyl)-pyrido[3,2-d]pyrimidine (74)

In a 25 mL flask, 202 mg (0.53 mmol, 1 equiv.) of 2-(4-hydroxyphenyl)-7-(3-(5-formyl)thiophyl)-pyrido[3,2-d]pyrimidine (73) are dissolved in 10 mL of MeOH. After a few minutes of stirring at room temperature, the mixture is cooled down to −10° C. At this temperature, sodium borohydride (10.12 mg, 0.3 mmol, 0.5 equiv.) is added. The whole is left with stirring at room temperature for one night. Next, the solvant is evaporated and the thereby obtained residue is purified on a silica gel chromatographic column (DCM/Et₃N 99/01, DCM/THF 9/1). The product 74 is obtained as a grey solid with a yield of 71%. MP>268° C.; ¹H NMR (400 MHz, DMSO-d₆) δ: 3.43 (s, 3H, CH₃), 4.72 (d, 2H, J=4 Hz, CH₂), 5.31 (s, 2H, CH₂), 5.64 (t, 1H, J=4 Hz, Hₜₕᵢₒₚ), 7.20 (d, 2H, J=8.0 Hz, H_{Arom}), 7.77 (s, 1H, Hₜₕᵢₒₚ), 8.38 (s, 1H, H₈), 8.50 (d, 2H, J=12.0 Hz, H_{Arom}), 8.62 (s, 1H, H₆), 9.47 (s, 1H, H₄), 9.63 (s, 1H, OH); ¹³C NMR (100 MHz, DMSO-d₆) δ: 55.79 (CH₃), 58.3 (CH₂), 93.7 (CH₂), 116.1 (2CH), 122.9 (CH), 124.6 (CH), 129.8 (CH), 129.9 (2CH), 130.3 (Cq), 135.5 (Cq), 136.2 (Cq), 137.2 (Cq), 146.8 (Cq), 148.6 (Cq), 150.7 (CH), 159.3 (Cq), 160.4 (Cq), 161.1 (CH). C₂₀H₁₇N₃O₃S, calculated m/z 380.1061 (M+1). found 380.1063 (M+1).

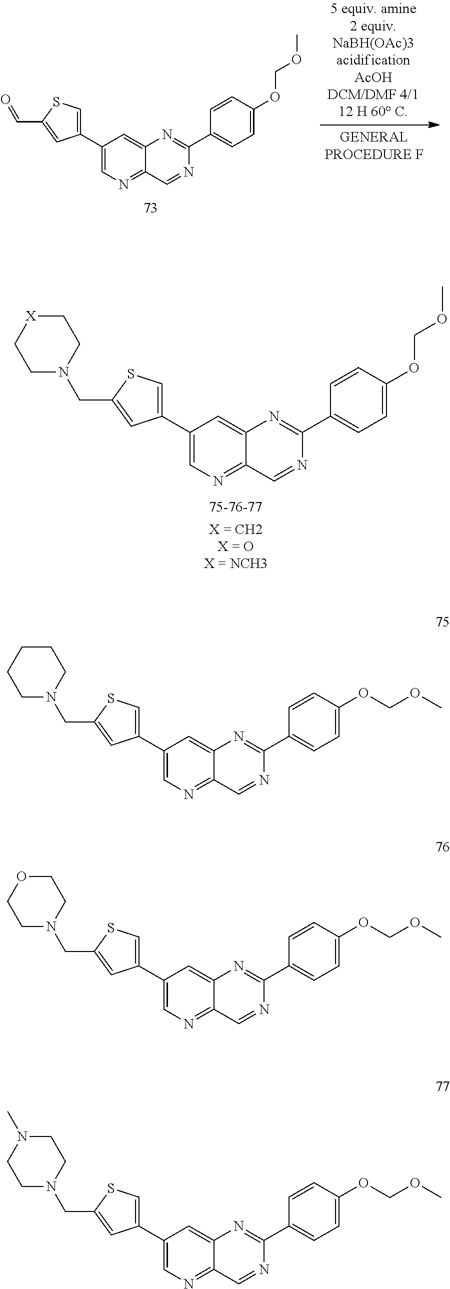

General Procedure F

In a flask, the compound 73 is dissolved in an excess of amine (5.0 equiv.) and of a mixture CH₂Cl₂/DMF (4/1) in the presence of NaBH(OAc)₃ (2.0 equiv.). After 12 h of stirring at 60° C., some acetic acid is added dropwise for neutralizing the mixture. After extraction with ethyl acetate and evaporation, the reaction crude product is subject to purification.

### 2-(4-Methoxymethoxy-phenyl)-7-(5-piperidin-1-ylmethyl-thiophen-3-yl)-pyrido[2,3-d]pyrimidine (75)

The product 75 is synthesized from 73 by following the general procedure F and then isolated after purification on a chromatographic silica gel column (CH₂Cl₂/MeOH 98/02) as a yellowish solid with a yield of 37%. MP: >268° C.; IR (ATR, Diamond, cm⁻¹) ν 2918, 1671, 1449, 1391, 1235, 1150, 1079, 1002, 852, 701; ¹H NMR (400 MHz, DMSO-d₆) δ: 1.39 (s, 2H, Hₚᵢₚ₄), 1.51 (s, 4H, Hₚᵢₚ₃ and Hₚᵢₚ₅), 2.41 (s, 4H, Hₚᵢₚ₂ and Hₚᵢₚ₆), 3.42 (s, 3H, OCH₃), 3.69 (s, 2H, NCH₂), 5.30 (s, 2H, OCH₂O), 7.19 (d, 2H, J=8.0 Hz, H_{Arom}), 7.75 (s, 1H, Hₜₕᵢₒₚ₅), 8.36 (s, 1H, Hₜₕᵢₒ₂), 8.49 (d, 2H, J=12.0 Hz, H_{Arom}), 8.61 (s, 1H, H₈), 9.45 (s, 1H, H₆), 9.61 (s, 1H, H₄); ¹³C NMR (100 MHz, DMSO-d₆) δ: 23.8 (CH₂), 25.5 (2CH₂), 53.7 (2CH₂), 55.7 (CH₃), 57.1 (CH₂), 93.7 (CH₂), 116.1 (2CH), 124.6 (CH), 125.0 (CH), 129.7 (CH), 129.9 (2CH), 130.8 (Cq), 135.5 (Cq), 136.0 (Cq), 137.2 (Cq), 144.9 (Cq), 146.8 (Cq), 150.7 (CH), 159.3 (Cq), 160.3 (Cq), 161.0 (CH). C₂₈H₂₇N₄O₂S, calculated m/z 447,1851 (M+1). found 447,1849 (M+1).

### 2-(4-Methoxymethoxy-phenyl)-7-(5-morpholin-4-ylmethyl-thiophen-3-yl)-pyrido[2,3-d]pyrimidine (76)

The product 76 is synthesized from 73 by following the general procedure F and then isolated after purification on a chromatographic silica gel column (CH₂Cl₂/MeOH 98/02) as a yellow solid with a yield of 34%. MP>268° C.; IR (ATR, Diamond, cm⁻¹) ν 2949, 1671, 1586, 1451, 1392, 1237, 1149, 1078, 990, 848, 741, 655; ¹H NMR (400 MHz, DMSO-d₆) δ: 2.45 (s, 4H, Hₘₒᵣₚₕ₂ and Hₘₒᵣₚ₆), 3.42 (5, 3H, CH₃), 3.60 (t, 4H, J=4.0 Hz, H_{Arom}), 3.73 (s, 2H, CH₂), 5.29 (s, 2H, CH₂), 7.18 (d, 2H, J=8.0 Hz, H_{Arom}), 7.76 (s, 1H, Hₜₕᵢₒₚ₅), 8.36 (s, 1H, Hₜₕᵢₒ₂), 8.47 (d, 2H, J=8.0 Hz, H_{Arom}), 8.58 (s, 1H, H₈), 9.43 (s, 1H, H₆), 9.58 (s, 1H, H₄); ¹³C NMR (100 MHz, DMSO-d₆) δ: 53.4 (2CH₂), 56.2 (CH₃), 57.1 (NCH₂), 66.6 (2CH₂), 94.1 (CH₂), 116.5 (2CH), 125.5 (CH), 125.7 (CH), 130.2 (CH), 130.4 (2CH), 130.8 (Cq), 135.8 (Cq), 136.5 (Cq), 137.6 (Cq), 144.1 (Cq), 147.2 (Cq), 151.1 (CH), 159.7 (Cq), 160.8 (Cq), 161.4 (CH); C₂₄H₂₄N₄O₃S, calculated m/z 449.1642 (M+1). found 449.1641 (M+1).

### 2-(4-Methoxymethoxy-phenyl)-7-[5-(4-methyl-piperazin-1-ylmethyl)-thiophen-3-yl]-pyrido[2,3-d]pyrimidine (77)

The product 77 is synthesized from 73 by following the general procedure F and then isolated after purification on a chromatographic silica gel column (CH₂Cl₂/Et₃N 99/01, CH₂Cl₂/THF 9/1) as a yellow solid with a yield of 36%. MP>268° C.; IR (ATR, Diamond, cm⁻¹) ν 2939, 1668, 1586, 1448, 1234, 1149, 1079, 1004, 843, 657; ¹H NMR (400 MHz, DMSO-d₆) δ: 2.17 (s, 3H, NCH₃), 2.36 (s, 8H, H_{piperazine}), 3.43 (s, 3H, CH₃), 3.73 (s, 2H, NCH₂), 5.30 (s, 2H, CH₂), 7.19 (d, 2H, J=8 Hz, H_{Arom}), 7.78 (s, 1H, Hₜₕᵢₒₚ₅), 8.37 (s, 1H, Hₜₕᵢₒₚ₂), 8.49 (d, 2H, J=8 Hz, H_{Arom}), 8.61 (s, 1H, H₈), 9.45 (s, 1H, H₆), 9.61 (s, 1H, H₄); ¹³C NMR (100 MHz, DMSO-d₆) δ: 45.5 (CH₃), 52.3 (2CH₂), 54.5 (2CH₂), 55.7 (CH₃), 56.3 (CH₂), 93.7 (CH₂), 116.1 (2CH), 124.9 (CH), 125.2 (CH), 129.8 (Cq), 129.9 (CH), 130.3 (2CH), 135.4 (Cq), 136.0 (Cq), 137.2 (Cq), 144.3 (Cq), 146.7 (Cq), 150.7 (CH), 159.3 (Cq), 160.3 (Cq), 161.0 (CH); C₂₈H₂₇N₈O₂S, calculated m/z 462.1960 (M+1). found 462.1958 (M+1).

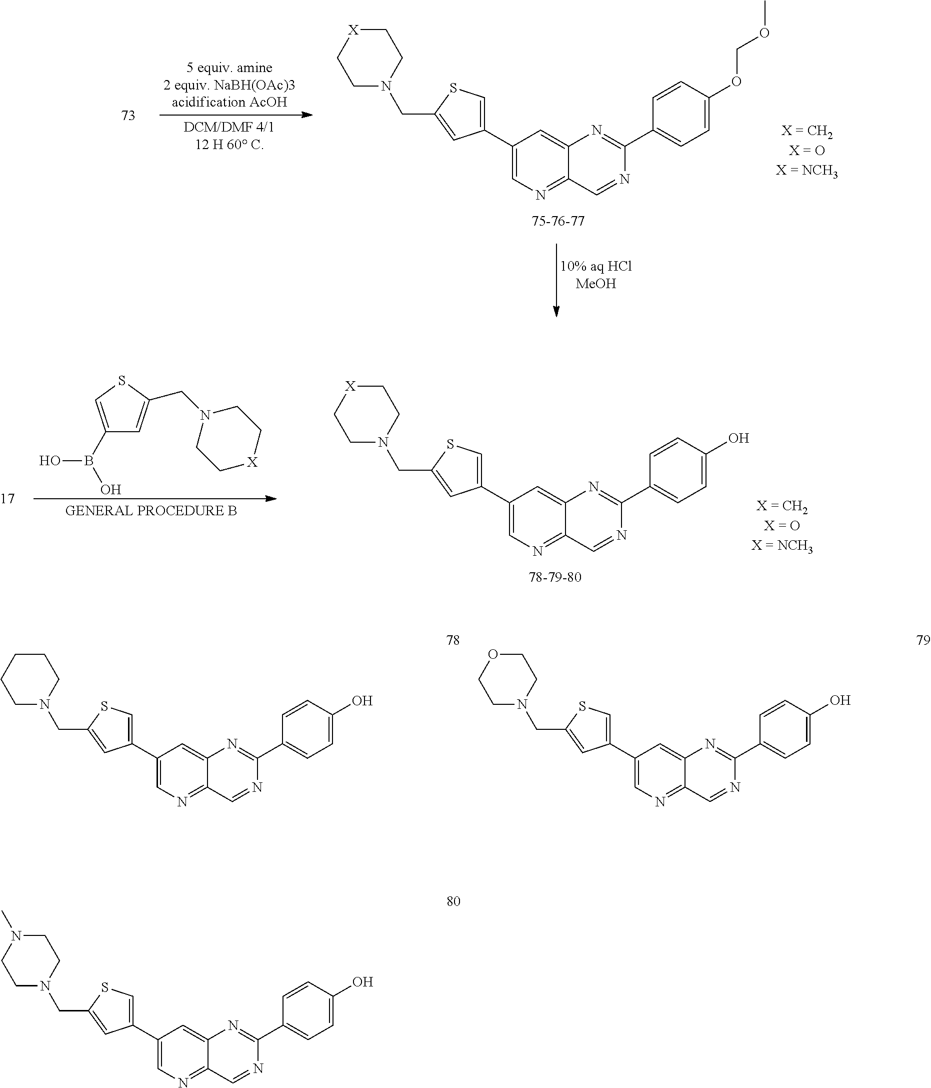

### Preparation of Boronic Acids (not Isolated)

5-formyl-3-thiophene boronic acid (100 mg, 0.64 mmol, 1 equiv.) is dissolved in 3 mL of DME, and then the adequate amine is added followed by a drop of acetic acid. The resulting mixture is stirred for 5 minutes at room temperature and then sodium triacetoxyborohydride is added. The solution is then brought to 60° C. for 5 h. The solvant and the excess of the amine are evaporated under reduced pressure. The thereby obtained residue is engaged without purification in a Suzuki type coupling with 7-chloro-2-(4-hydroxyphenyl)pyrido[2,3-c]pyrimidines (17), following the general procedure B.

### 4-[7-(5-Piperidin-1-ylmethyl-thiophen-3-yl)-pyrido[2,3-d]pyrimidin-2-yl]-phenol (78)

The product 78 is synthesized from 17 by following the general procedure B for 10 mins and then isolated after purification on a chromatographic silica gel column (CH₂Cl₂/Et₃N 9/1, CH₂Cl₂/THF 9/1) as a yellowish solid with a yield of 47%. It may also be obtained from 75 by treatment with aqueous 10% hydrochloric acid in MeOH with quantitative yield. MP>268° C.; IR (ATR, Diamond, cm⁻¹) ν 3089, 2948, 2545, 1577, 1451, 1158, 939, 847, 810, 657; ¹H NMR (400 MHz, DMSO-d₆) δ: 1.39 (s, 2H, Hₚᵢₚ₄), 1.51 (s, 4H, Hₚᵢₚ₃ and Hₚᵢₚ₅) 2.43 (s, 4H, Hₚᵢₚ₂ and Hₚᵢₚ₆), 3.71 (s, 2H, NCH₂), 6.93 (d, 2H, J=8.0 Hz, H_{Arom}), 7.75 (s, 1H, Hₜₕᵢₒₚ₅), 8.35 (s, 1H, Hₜₕᵢₒ₂), 8.40 (d, 2H, J=8.0 Hz, H_{Arom}), 8.57 (s, 1H, H₈), 9.42 (s, 1H, H₆), 9.58 (s, 1H, H₄), 10.13 (s, 1H, OH); ¹³C NMR (100 MHz, DMSO-d₆) δ: 23.8 (CH₂), 25.5 (2CH₂), 53.7 (2CH₂), 57.0 (CH₂), 115.6 (2CH), 124.8 (CH), 125.1 (CH), 127.9 (Cq), 129.8 (CH), 130.3 (2CH), 135.5 (Cq), 136.2 (Cq), 137.2 (Cq), 144.6 (Cq), 146.8 (Cq), 150.4 (CH), 160.5 (Cq), 160.9 (Cq), 161.0 (CH); C₂₃H₂₂N₄OS, calculated m/z 403,1589 (M+1). found 403,1587 (M+1).

### 4-[7-(5-Morpholin-4-ylmethyl-thiophen-3-yl)-pyrido[2,3-d]pyrimidin-2-yl]-phenol (79)

The product 79 is synthesized from 17 by following the general procedure B for 10 mins and then isolated after purification on a chromatographic silica gel column (CH₂Cl₂/Et₃N 9/1, CH₂Cl₂/THF 9/1) as a brown solid with a yield of 30%. It may also be obtained from 76 by treatment with aqueous 10% hydrochloric acid in MeOH with quantitative yield. MP>268° C.; IR (ATR, Diamond, cm⁻¹) ν 3096, 2943, 2674, 1664, 1571, 1443, 1390, 1260, 1153, 1098, 849, 803, 706; ¹H NMR (400 MHz, DMSO-d₆) δ: 2.46 (s, 4H, Hₘₒᵣₚₕ₂ and Hₘₒᵣₚₕ₆), 3.58 (4H, Hₘₒᵣₚₕ₃ and Hₘₒᵣₚₕ₅), 3.74 (s, 2H, CH₂), 6.93 (d, 2H, J=8.0 Hz, H_{Arom}), 7.75 (s, 1H, Hₜₕᵢₒₚ₅), 8.36-8.43 (m, 3H, H_{Arom} and Hₜₕ 8.57 (s, 1H, H₈), 9.42 (s, 1H, H₆), 9.57 (s, 1H, H₄), 10.08 (s, 1H, OH); ¹³C NMR (100 MHz, DMSO-d₆) δ: 52.9 (2CH₂), 56.7 (CH₂), 66.1 (2CH₂), 115.6 (2CH), 125.1 (CH), 125.2 (CH), 127.8 (Cq), 129.7 (CH), 130.2 (2CH), 135.3 (Cq), 136.1 (Cq), 137.1 (Cq), 143.7 (Cq), 146.7 (Cq), 150.3 (CH), 160.4 (Cq), 160.8 (Cq), 160.9 (CH); C₂₂H₂₀N₄O₂S, calculated m/z 405,1383 (M+1). found 405.1379 (M+1).

### 4-{7[4-(4-Methyl-piperazin-1-ylmethyl-thiophen-3-yl]-pyrido[2,3-d]pyrimidin-2-yl]-phenol (80)

The product 80 is synthesized from 17 by following the general procedure B for 10 mins and then isolated after purification on a chromatographic silica gel column (EtOAc/Et₃N 9/1, EtOAc/MeOH 95/05) as a yellow solid with a yield of 29%. It may also be obtained from 77 by treatment with aqueous 10% hydrochloric acid in MeOH with quantitative yield. MP>268° C.; IR (ATR, Diamond, cm⁻¹) ν 2939, 1660, 1571, 1462, 1378, 1244, 1152, 1001, 844, 799, 658; ¹H NMR (400 MHz, DMSO-d₆) δ: 2.18 (s, 3H, NCH₃), 2.37 (s, 8H, H_{piperazine}), 3.74 (5, 2H, NCH₂), 6.94 (d, 2H, J=8 MHz, H_{Arom}), 7.79 (s, 1H, Hₜₕᵢₒₚ₅), 8.38 (s, 1H, Hₜₕᵢₒₚ₂), 8.41 (d, 2H, J=12 MHz, H_{Arom}), 8.59 (s, 1H, H₈), 9.44 (s, 1H, H₆), 9.59 (s, 1H, H₄), 10.1 (s, 1H, OH); ¹³C NMR (100 MHz, DMSO-d₆) δ: 46.0 (CH₃), 52.7 (2CH₂), 55.0 (2CH₂), 56.8 (CH₂), 116.1 (2CH), 125.4 (CH), 125.6 (CH), 128.3 (Cq), 130.2 (CH), 130.7 (2CH), 135.8 (Cq), 136.6 (Cq), 137.6 (Cq), 144.8 (Cq), 147.2 (Cq), 150.8 (CH), 160.9 (Cq), 161.3 (Cq), 161.4 (CH). C₂₃H₂₃N₈OS, calculated m/z 418.1699 (M+1). found 418.1696 (M+1).

### 1.9. Aminations in positions C-2 and then in C-4 from 2

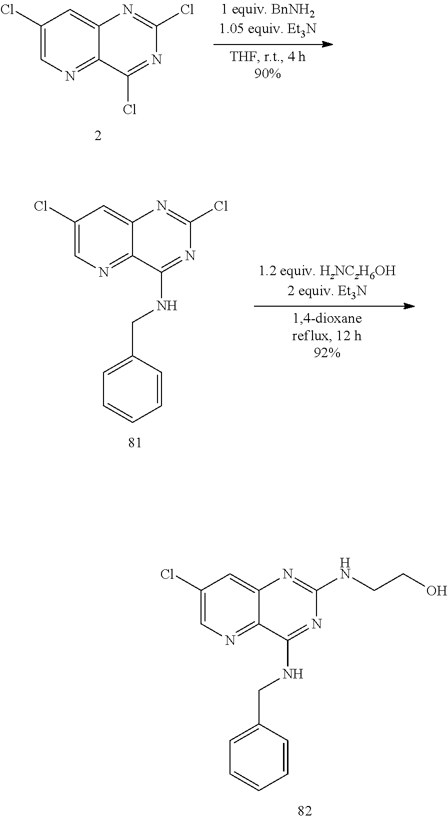 [SMILES: Clc1cnc2c(Cl)nc(Cl)nc2c1 ; Clc1cnc2c(NCc3ccccc3)nc(Cl)nc2c1 ; OCCNc1nc(NCc2ccccc2)c2ncc(Cl)cc2n1]

### 4-Benzylamino-2,7-dichloro-pyrido[3,2-d]pyrimidine (81)

In a 250 mL flask, 2 g (8.53 mmol 1.0 equiv.) of 2,4,7-trichloropyrido[3,2-c]pyrimidine 2 are dissolved in 100 mL of anhydrous THF and then 914 mg (8.53 mmol, 1.0 equiv.) of benzylamine and 863 mg (7.76 mmol, 1.05 equiv.) of triethylamine are added respectively. The whole is maintained at room temperature for 4 hours. Next, the THF is evaporated and then the obtained residue is taken up with water and extracted with dichloromethane. The organic phase is dried on MgSO₄ and then concentrated under reduced pressure. The compound 81 is obtained, after purification on a chromatographic silica gel column (petroleum ether/CH₂Cl₂, 5/5) as a white solid with a yield of 90%. MP: 169-170° C.; IR (ATR, Diamond, cm⁻¹) ν 3036, 2156, 1601, 1572, 1524, 1438, 1297, 1130, 1045, 880; ¹H NMR (250 MHz, CDCl₃) δ: 4.85 (d, 2H, J=5.8 Hz, CH₂), 7.33-7.39 (m, 5H, H_{Ph}), 7.48 (sl, 1H, NH), 8.00 (d, 1H, J=2.1 Hz, H₈), 8.56 (d, 1H, J=2.1 Hz, H₆); ¹³C NMR (62.5 MHz, CDCl₃) δ: 45.3 (CH₂), 128.1 (CH), 128.2 (2CH), 128.8 (Cq), 129.1 (2CH), 133.6 (CH), 136.3 (Cq), 136.9 (Cq), 146.1 (Cq), 147.8 (CH), 159.7 (Cq), 160.4 (Cq). HRMS (EI-MS): C₁₄H₁₈³⁵Cl₂N₄, calculated m/z 305.0361. found m/z 305.0365.

### 4-Benzylamino-7-chloro-2-(2-hydroxyethylamino)-pyrido[3,2-d]pyrimidine (82)

In a 100 mL flask, 1.5 g (4.91 mmol, 1.0 equiv.) of 4-benzylamino-2,7-dichloro-pyrido[3,2-c]pyrimidine 81 are dissolved in 60 mL of 1,4-dioxane for analysis and then 360 mg (5.90 mmol, 1.2 equiv.) of ethanolamine and 994 mg (9.83 mmol, 2.0 equiv.) of triethylamine are added respectively. The whole is refluxed for 12 hours. Next, the 1,4-dioxane is evaporated and the obtained residue is then taken up with water (20 mL) and extracted with dichloromethane (2×20 mL). The organic phase is dried on MgSO₄ and then concentrated under reduced pressure. The compound 82 is obtained, after purification on a chromatographic silica gel column (CH₂Cl₂/MeOH, 95/5) as a yellow solid with a yield of 92%. MP: 106-107° C.; IR (ATR, Diamond, cm⁻¹) ν 3406, 2843, 1609, 1568, 1515, 1445, 1312, 1159, 1067, 893; ¹H NMR (250 MHz, CDCl₃) δ: 3.57-3.63 (m, 2H, CH₂), 3.81-3.84 (m, 2H, CH₂), 4.58 (sl, 1H, OH), 4.71 (d, 1H, J=5.9 Hz, CH₂Ph), 5.74 (sl, 1H, NH), 7.26-7.35 (m, 6H, H_{Ph} and NH), 7.63 (d, 1H, J=2.0 Hz, H₈), 8.16 (d, 1H, J=2.0 Hz, H₆); ¹³C NMR (62.5 MHz, CDCl₃) δ: 44.6 (CH₂), 44.9 (CH₂), 63.7 (CH₂), 127.3 (Cq), 127.7 (2CH), 127.8 (2CH), 128.8 (CH), 130.6 (CH), 135.5 (Cq), 137.9 (Cq), 142.3 (CH), 146.5 (Cq), 159.6 (Cq), 160.9 (Cq); HRMS (EI-MS): C₁₆H₁₆³⁵ClN₆O, calculated m/z 330.1122 (M+1). found m/z 330.1107 (M+1).

### 1.10. Aminations of the Backbone in Positions C-2, C-4 and in C-7

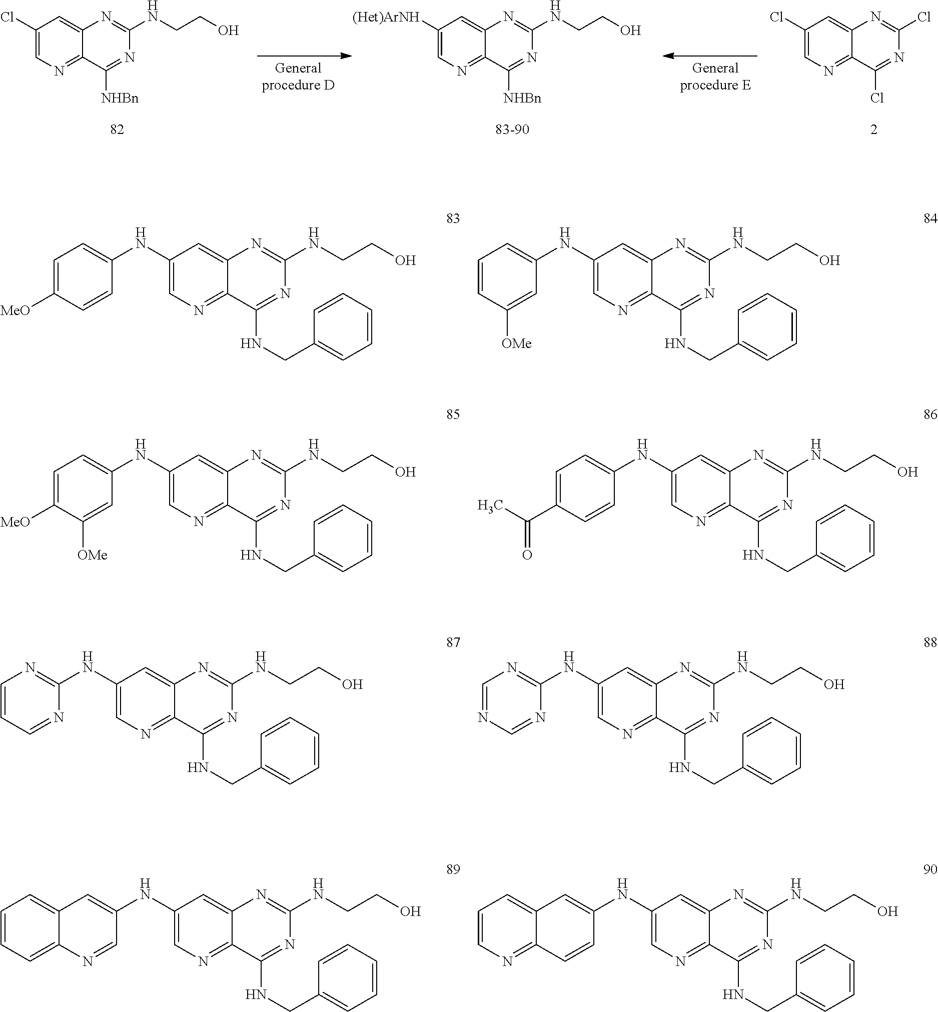

General Procedure D:

Under an argon atmosphere, in a vial, the 4-benzylamino-7-chloro-2-(2-hydroxyethylamino)-pyrido[3,2-d]pyrimidine 82 is dissolved in dioxane for analysis and then 1.2 equiv. of amine, 2.0 equiv. of potassium carbonate, 0.1 equiv. of palladium acetate de palladium and 0.2 equiv. of Xantphos are added. The whole is brought to 140° C. with microwave irradiation for 50 minutes. The dioxane is evaporated and then the obtained residue is purified on a silica gel chromatographic column.

General Procedure E:

Under an argon atmosphere, in a vial, 2,4,7-trichloropyrido[3,2-d]pyrimidine 2 is dissolved in 2 mL of dioxane for analysis, 1 equiv. of benzylamine, 3.0 equiv. of triethylamine are added respectively. After 5 minutes at room temperature, 5.0 equiv. of ethanolamine are introduced and the mixture is brought to 140° C. with microwave irradiation for one hour. Finally, 1.2 equiv. of the desired heteroaromatic amine in position 7, 2.0 equiv. of potassium carbonate, 0.1 equiv. of palladium acetate and 0.2 equiv. of Xantphos are added. The whole is maintained at 140° C. with microwave irradiation for one hour. The dioxane is evaporated and the obtained residue is then purified on a silica gel chromatographic column.

### 4-Benzylamino-2-(2-hydroxyethylamino)-7-(4-methoxyphenylamino)-pyrido[3,2-d]pyrimidine (83)

The product 83 is synthesized from 82 by following the general procedure D with a yield of 72% or from 2 by following the general procedure E with a yield of 64% and then purified on a chromatographic silica gel column (CH₂Cl₂/MeOH, 95/5) as an orange solid. MP: 126-127° C.; IR (ATR, Diamond, cm⁻¹) ν 2926, 1564, 1503, 1454, 1414, 1326, 1237, 1175, 1028, 816; ¹H NMR (250 MHz, CDCl₃) δ: 3.57-3.61 (m, 2H, CH₂), 3.80-3.83 (m, 5H, CH₂ and OCH₃), 4.73 (d, 2H, J=5.9 Hz, CH₂Ph), 5.48 (sl, 1H, OH), 5.84 (sl, 1H, NH), 6.88-6.94 (m, 3H, H_{Arom} and H₈), 7.06 (sl, 1H, NH), 7.14 (d, 2H, J=8.9 Hz, H_{Arom}), 7.29-7.37 (m, 5H, H_{Ph}), 7.93 (d, 1H, J=2.5 Hz, H₆); ¹³C NMR (100 MHz, CDCl₃) δ: 43.0 (CH₂), 43.5 (CH₂), 55.1 (CH₃), 60.4 (CH₂), 107.8 (CH), 114.6 (2CH), 120.6 (Cq), 122.4 (2CH), 126.3 (CH), 127.1 (2CH), 127.8 (2CH), 133.7 (Cq), 134.6 (CH), 139.5 (Cq), 145.4 (Cq), 146.8 (Cq), 155.2 (Cq), 158.8 (Cq), 159.1 (Cq). (Experiment at 80° C.); HRMS (EI-MS): C₂₃H₂₄N₆O₂, calculated m/z 417.2039 (M+1). found m/z 417.2033 (M+1).

### 4-Benzylamino-2-(2-hydroxyethylamino)-7-(3-methoxyphenylamino)-pyrido[3,2-d]pyrimidine (84)

The product 84 is synthesized from 82 by following the general procedure D with a yield of 76% or from 2 by following the general procedure E with a yield of 75% and then purified on a chromatographic silica gel column (AcOEt/MeOH, 95/05) as a yellow solid. MP: 110-111° C.; IR (ATR, Diamond, cm⁻¹) ν 3252, 2930, 2290, 1568, 1490, 1320, 1230, 1152, 1048, 846; ¹H NMR (250 MHz, DMSO-d₆) δ: 3.33-3.37 (m, 2H, CH₂), 3.47-3.52 (m, 2H, CH₂), 3.75 (s, 3H, OCH₃), 4.65 (d, 2H, J=6.3 Hz, CH₂Ph), 6.50 (t, 1H, J=6.3 Hz, NH), 6.58 (dd, 1H, J=2.0 Hz, 8.1 Hz, H_{Arom}), 6.71-6.73 (m, 1H, H_{Arom}), 6.80 (d, 1H, J=8.1 Hz, H_{Arom}), 7.06 (d, 1H, J=2.0 Hz, H_{Arom}), 7.18-7.39 (m, 6H, H_{Ph} and H₈), 8.09 (d, 1H, J=2.4 Hz, H₆), 8.27 (sl, 1H, NH), 8.75 (s, 1H, NH); ¹³C NMR (100 MHz, DMSO-d₆) δ: 42.9 (CH₂), 43.5 (CH₂), 54.7 (CH₃), 60.4 (CH₂), 104.8 (CH), 107.2 (CH), 110.9 (CH), 111.1 (CH), 121.6 (Cq), 126.2 (CH), 127.1 (2CH), 127.7 (2CH), 129.7 (CH), 135.1 (CH), 139.5 (Cq), 142.6 (Cq), 143.5 (Cq), 147.4 (Cq), 158.8 (Cq), 159.6 (Cq), 160.1 (Cq). (Experiment at 80° C.); HRMS (EI-MS): C₂₃H₂₄N₆O₂, calculated m/z 417.2039 (M+1). found m/z 439.2039 (M+1).

### 4-Benzylamino-7-(3,4-dimethoxyphenylamino)-2-(2-hydroxyethylamino)-pyrido[3,2-d]pyrimidine (85)

The product 85 is synthesized from 82 by following the general procedure D with a yield of 71% or from 2 by following the general procedure E with a yield of 68% and then purified on a chromatographic silica gel column (AcOEt/MeOH, 99.5/0.5) as a yellow solid. MP: 149-150° C.; IR (ATR, Diamond, cm⁻¹) ν 3837, 1585, 1504, 1504, 1449, 1235, 1172, 1058, 800, 733; ¹H NMR (250 MHz, CDCl₃) δ: 3.56 (sl, 2H, CH₂), 3.77-3.82 (m, 5H, CH₂ and OCH₃), 3.85 (s, 3H, OCH₃), 4.69 (d, 2H, J=5.9 Hz, CH₂Ph), 5.58 (sl, 1H, NH), 6.30 (sl, 1H, NH), 6.69-6.81 (m, 3H, H_{Ph} and H_{Arom}), 6.97 (d, 1H, J=2.4 Hz, H₈), 7.06 (t, 1H, J=5.9 Hz, NH), 7.29-7.34 (m, 5H, H_{Ph} and H_{Arom}), 7.92 (d, 1H, J=2.4 Hz, H₆); ¹³C NMR (62.5 MHz, DMSO-d₆) δ: 44.5 (CH₂), 45.3 (CH₂), 56.1 (CH₃), 56.3 (CH₃), 64.8 (CH₂), 107.1 (CH), 110.3 (CH), 112.1 (CH), 114.6 (CH), 121.9 (Cq), 127.5 (CH), 127.8 (2CH), 128.8 (2CH), 133.3 (Cq), 135.2 (CH), 138.5 (Cq), 145.7 (Cq), 146.3 (Cq), 146.9 (Cq), 149.8 (Cq), 159.5 (Cq), 160.9 (Cq). (Experiment at 80° C.); HRMS (EI-MS): C₂₄H₂₆N₆O₃, calculated m/z 447.2145 (M+1). found m/z 447.2146 (M+1).

### 4-Benzylamino-7-(4-acetylphenylamino)-2-(2-hydroxyethylamino)-pyrido[3,2-d]pyrimidine (86)

The product 86 is synthesized from 82 by following the general procedure D with a yield of 78% or from 2 by following the general procedure E with a yield of 70% and then purified on a chromatographic silica gel column (CH₂Cl₂/MeOH, 95/5) as a yellow solid. MP: 125-126° C.; IR (ATR, Diamond, cm⁻¹) ν 1645, 1570, 1511, 1467, 1342, 1287, 1180, 1062, 829, 726; ¹H NMR (250 MHz, DMSO-d₆) δ: 2.50 (s, 3H, CH₃), 3.33-3.39 (m, 2H, CH₂), 3.48-3.50 (m, 2H, CH₂), 4.66 (d, 2H, J=6.3 Hz, CH₂Ph), 4.93 (sl, 1H, OH), 6.59 (t, 1H, J=6.3 Hz, NH), 7.19-7.40 (m, 8H, H_{Ph}, H_{Arom} and H₈), 7.92 (d, 1H, J=8.6 Hz, H_{Arom}), 8.17 (d, 1H, J=2.4 Hz, H₆), 8.36 (sl, 1H, NH), 9.26 (s, 1H, NH); ¹³C NMR (100 MHz, DMSO-d₆) δ: 25.6 (CH₃), 43.1 (CH₂), 43.5 (CH₂), 60.2 (CH₂), 116.0 (CH), 126.3 (CH), 127.2 (CH), 127.8 (CH), 129.6 (Cq), 129.7 (CH), 136.1 (CH), 139.2 (Cq), 141.9 (Cq), 145.9 (Cq), 146.2 (Cq), 158.7 (2Cq), 158.9 (Cq), 195.3 (Cq). (Experiment at 80° C.); HRMS (EI-MS): C₂₄H₂₄N₆O₂, calculated m/z 429.2039 (M+1). found m/z 429.2047 (M+1).

### 4-Benzylamino-2-(2-hydroxyethylamino)-7-(2-pyrimidinylamino)-pyrido[3,2-d]pyrimidine (87)

The product 87 is synthesized from 82 by following the general procedure D with a yield of 73% or from 2 by following the general procedure E with a yield of 71% and then purified on a chromatographic silica gel column (AcOEt/MeOH, 90/10) as a yellow solid. MP: 141-142° C.; IR (ATR, Diamond, cm⁻¹) ν 3247, 2935, 2290, 1566, 1517, 1407, 1343, 1200, 1051, 882; ¹H NMR (250 MHz, CDCl₃) δ: 3.62-3.63 (m, 2H, CH₂), 3.84-3.88 (m, 2H, CH₂), 4.54 (sl, 1H, OH), 4.70 (d, 2H, J=5.9 Hz, CH₂Ph), 5.86 (sl, 1H, NH), 6.75 (t, 1H, J=4.8 Hz, H_{hét}), 7.23 (sl, 1H, NH), 7.27-7.34 (m, 5H, H_{Ph}), 8.21 (sl, 1H, NH), 8.31 (d, 1H, J=2.3 Hz, H₈), 8.34 (d, 1H, J=2.3 Hz, H₆), 8.42 (d, 2H, J=4.8 Hz, Hₕₑₜ); ¹³C NMR (100 MHz, DMSO-d₆) δ: 43.0 (CH₂), 43.5 (CH₂), 60.4 (CH₂), 113.1 (CH), 116.3 (CH), 122.8 (Cq), 126.3 (CH), 127.1 (2CH), 127.8 (2CH), 136.1 (CH), 139.4 (Cq), 140.0 (Cq), 146.6 (Cq), 157.6 (2CH), 158.9 (Cq), 159.4 (Cq), 159.5 (Cq). (Experiment at 80° C.); HRMS (EI-MS): C₂₀H₂₀N₈O, calculated m/z 389.1838 (M+1). found m/z 389.1841 (M+1).

### 4-Benzylamino-2-(2-hydroxyethylamino)-7-(1,3,5-triazinylamino)-pyrido[3,2-d]pyrimidine (88)

The product 88 is synthesized from 82 by following the general procedure D with a yield of 81% or from 2 by following the general procedure E with a yield of 67% and then purified on a chromatographic silica gel column (AcOEt/MeOH, 90/10) as a yellow solid. MP: 202-203° C. IR (ATR, Diamond, cm⁻¹) ν 3416, 2904, 2280, 1619, 1573, 1430, 1307, 1205, 1067, 811; ¹H NMR (250 MHz, DMSO-d₆) δ: 3.37-3.41 (m, 2H, CH₂), 3.50-3.52 (m, 2H, CH₂), 4.68 (d, 2H, J=5.9 Hz, CH₂Ph), 6.71 (sl, 1H, NH), 7.21-7.40 (m, 5H, H_{Ph}), 8.22 (sl, 1H, H₈), 8.45 (sl, 1H, NH), 8.56 (d, 1H, J=2.3 Hz, H₆), 8.87 (s, 2H, H_{hét}), 10.70 (sl, 1H, NH); ¹³C NMR (100 MHz, DMSO-d₆) δ: 43.0 (CH₂), 43.5 (CH₂), 60.2 (CH₂), 119.5 (CH), 124.1 (Cq), 126.2 (CH), 127.1 (2CH), 127.7 (2CH), 136.3 (CH), 138.1 (Cq), 139.2 (Cq), 146.6 (Cq), 158.8 (Cq), 159.6 (Cq), 163.0 (Cq), 165.8 (2CH). (Experiment at 80° C.); HRMS (EI-MS): C₁₈H₁₈N₈O, calculated m/z 390.1791 (M+1). found m/z 390.1807 (M+1).

### 4-Benzylamino-2-(2-hydroxyethylamino)-7-(3-quinolinamino)-pyrido[3,2-d]pyrimidine (89)

The product 89 is synthesized from 82 by following the general procedure D with a yield of 64% or from 2 by following the general procedure E with a yield of 72% and then purified on a chromatographic silica gel column (AcOEt/MeOH, 99.5/0.5) as a yellow solid. MP: 154-155° C.; IR (ATR, Diamond, cm⁻¹) ν 3242, 2924, 1645, 1565, 1452, 1345, 1234, 1123, 1046, 826; ¹H NMR (400 MHz, DMSO-d₆) δ: 3.60-3.64 (m, 2H, CH₂), 3.83-3.85 (m, 2H, CH₂), 4.75 (d, 2H, J=5.9 Hz, CH₂Ph), 5.49 (sl, 1H, OH), 6.42 (sl, 1H, NH), 7.11 (t, 1H, J=5.9 Hz, NH), 7.28-7.32 (m, 2H, H_{Ph}), 7.34-7.40 (m, 4H, H_{Ph} and H_{Arom}), 7.52 (t, 1H, J=7.0 Hz, H_{Arom}), 7.60 (dt, 1H, J=1.4 Hz, J=7.0 Hz, H_{Arom}), 7.72 (d, 1H, J=8.0 Hz, H_{Arom}), 7.93 (d, 1H, J=2.4 Hz, H₈), 8.05 (d, 1H, J=8.4 Hz, H_{Arom}), 8.13 (d, 1H, J=2.4 Hz, H₆), 8.75 (d, 1H, J=2.6 Hz, H_{Arom}); ¹³C NMR (100 MHz, DMSO-d₆) δ: 43.1 (CH₂), 43.5 (CH₂), 60.1 (CH₂), 118.8 (CH), 121.4 (Cq), 126.3 (CH), 126.51 (CH), 126.54 (2CH), 126.58 (CH), 127.2 (2CH), 127.8 (2CH), 127.9 (Cq), 128.2 (CH), 135.0 (Cq), 135.5 (CH), 139.1 (Cq), 143.2 (Cq), 143.3 (Cq), 145.4 (CH), 158.2 (Cq), 158.7 (2Cq). (Experiment at 80° C.); HRMS (EI-MS): C₂₅H₂₃N₇O, calculated m/z 438.2042 (M+1). found m/z 438.2044 (M+1).

### 4-Benzylamino-2-(2-hydroxyethylamino)-7-(6-quinolinamino)-pyrido[3,2-d]pyrimidine (90)

The product 90 is synthesized from 82 by following the general procedure D with a yield of 77% or from 2 by following the general procedure E with a yield of 69% and then purified on a chromatographic silica gel column (AcOEt/MeOH/Et₃N, 94/5/1) as a yellow solid. MP: 203-204° C. IR (ATR, Diamond, cm⁻¹) ν 3427, 2915, 2172, 1614, 1565, 1503, 1382, 1243, 1026, 846; ¹H NMR (400 MHz, DMSO-d₆) δ: 3.35-3.40 (m, 2H, CH₂), 3.51 (sl, 2H, CH₂), 4.67 (d, 2H, J=6.3 Hz, CH₂Ph), 6.67 (sl, 1H, NH), 7.23 (t, 1H, J=7.2 Hz, H_{Quino}), 7.29-7.33 (m, 3H, H_{Ph}), 7.38-7.40 (m, 2H, H_{Ph}), 7.45 (dd, 1H, J=4.2, 8.3 Hz, H_{Quino}), 7.62 (dd, 1H, J=2.3, 9.0 Hz, H_{Quino}) 7.70 (d, 1 J=2.2 Hz, H₈), 7.98 (d, 1H, J=9.0 Hz, H_{Quino}), 8.24, (d, 1H, J=2.2 Hz, H₆), 8.27 (d, 1H, J=8.3 Hz, H_{Quino}), 8.31 (sl, 1H, NH), 8.72 (dd, 1H, J=1.4 Hz, 4.2 Hz, H_{Quino}), 9.19 (sl, 1H, NH); ¹³C NMR (100 MHz, DMSO-d₆) δ: 43.0 (CH₂), 43.5 (CH₂), 60.3 (CH₂), 111.2 (CH), 111.8 (CH), 121.2 (CH), 121.9 (Cq), 123.6 (CH), 126.3 (CH), 127.1 (2CH), 127.8 (2CH), 128.7 (Cq), 129.9 (CH), 134.1 (CH), 135.5 (CH), 139.3 (Cq), 139.5 (Cq), 143.1 (Cq), 144.0 (Cq), 146.6 (Cq), 147.7 (CH), 158.8 (Cq), 159.1 (Cq). (Experiment at 80° C.); HRMS (EI-MS): C₂₅H₂₃N₇O, calculated m/z 438.2042 (M+1). found m/z 438.2047 (M+1).

### 2. Biological Results

2.1. Dosage Methods

The activities for inhibiting protein kinases of the compounds of the invention are tested according to the following general procedure:

Buffer Solutions

Buffer A: 10 mM MgCl₂, 1 mM EGTA, 1 mM DTT, 25 mM Tris-HCl pH 7.5 and 50 μg heparin/mL.

Buffer C: 60 mM β-glycerophosphate, 15 mM p-nitrophenyl-phosphate, 25 mM Mops (pH 7.2), 5 mM EGTA, 15 mM MgCl₂, 1 mM DTT, 1 mM of sodium vanadate

Preparation of the Kinases and Dosages

The kinase activities are dosed in buffers A or C, at 30° C., at a final concentration of ATP of 15 μM. The values of the blanks were subtracted and the activities are expressed as a % of the maximum activity, i.e. in the absence of inhibitors. The controls were carried out with suitable dilutions of DMSO.

CDK5 (humane, recombinant) was prepared as described earlier (Leclerc, S.; Garnier, M.; Hoessel, R.; Marko, D.; Bibb, J. A.; Snyder, G. L.; Greengard, P.; Biernat, J.; Mandelkow, E.-M.; Eisenbrand, G.; Meijer, L. Indirubins inhibit glycogen synthase kinase-3β and CDK5/p25, two kinases involved in abnormal tau phosphorylation in Alzheimer's disease—A property common to most CDK inhibitors? J. Biol. Chem. 2001, 276, 251-260; Bach S, Knockaert M, Reinhardt J, Lozach O, Schmitt S, Baratte B et al. (2005). Roscovitine targets, protein kinases and pyridoxal kinase. J. Biol Chem 280: 31208-31219). Its kinase activity was analyzed in the buffer C, with 1 mg of histone H1/mL, in the presence of 15 μM of [γ-³³P] ATP (3,000 Ci/mmol; 10 mCi/mL) in a final volume of 30 μl. After 30 mins of incubation at 30° C., 25 μl of supernatant aliquots were deposited on 2.5×3 cm pieces of phosphocellulose Whatman P81 paper, and after 20 seconds, the filters were washed five times (for at least 5 minutes every time) in a solution of 10 mL phosphoric acid/liter of water. The wet filters were counted in the presence of 1 mL of ACS scintillator fluid (Amersham).

GSK-3αβ (from pig brain, native) was assayed by using a specific substrate of GSK-3 (GS-1: YRRAAVPPSPSLSRHSSPHQSpEDEEE)(Sp represents a phosphorylated serine) (Primot, A., Baratte, B., Gompel, M., Borgne, A., Liabeuf, S., Romette, J. L., Costantini, F. and Meijer, L., 2000. Purification of GSK-3 by affinity chromatography on immobilized axin. Protein Expr. & Purif. 20 (3), 394-404). GS-1 was synthesized by Millegen (Labège, France). Its kinase activity was analyzed in the buffer A, with 1 mg of histone H1/mL, in the presence of 15 μM of [γ-³³P] ATP (3,000 Ci/mmol; 10 mCi/mL) in a final volume of 30 μl. After 30 mins of incubation at 30° C., 25 μl of supernatant aliquots were deposited on 2.5×3 cm pieces of phosphocellulose Whatman P81papier, and after 20 seconds, the filters were washed five times (for at least 5 minutes every time) in a solution of 10 mL of phosphoric acid/liter of water. The wet filters were counted in the presence of 1 mL of ACS scintillator fluid (Amersham).

DYRK1A (humane, recombinant, expressed in E. coli as a fusion protein GST) was purified by affinity chromatography on glutathione-agarose beads and measured in buffer A (+0.5 mg of bovine albumin serum/mL) with the Woodtide substrate (1.5 μg/dosage).

In Vivo Dosages on Human cells (Huh7, Caco, MDA-MB 231, HCT 116, PC3, NCl, Fibroblast)

Cytotoxicity: This method is based on an automated imaging analysis. 4.10³ cells were cultivated on 96-well plates and thus left for 24 hours so that they may bind, be distributed and proliferate.

These cells were then exposed for 24 h and 48 h to increasing concentrations of the compounds of the invention, from 0.1 to 25 μM in a final volume of 80 μL of culture medium. The cells are then set with a 4% paraformaldehyde solution and the nuclei are colored with Hoechst 3342 and counted according to automated imaging quantification.

All the cell lines were cultivated in a DMEM or RPMI (Invitrogen) medium. All these media were completed with antibiotics (penicillin-streptomycin)(Lonza) and 10% by volume of foetal calf serum (Invitrogen). The cells were cultivated at 37° C. with 5% of CO₂. The treatments with the molecules to be tested were carried out with increasing concentrations. The control experiments were also carried out with increasing concentrations. The control experiments were also conducted by using suitable dilutions of DMSO (maximum 1% DMSO). Cell viability was determined by measuring the reduction of MTS as described in the article of Ribas J, Boix J. (2004). Cell differentiation caspase inhibition and macromolecular synthesis blockage but not BCL-2 or BCL-XL proteins protect SH-SY5Y cells from apoptosis triggered by two CDK inhibitory drugs. Exp. Cell Res., 295: 9-24.

2.2. Results

The obtained results are indicated in the tables hereafter.

| | | Human cells | | | | | | | | | |
|---|---|---|---|---|---|---|---|---|---|---|---|
| | Species | Huh7 | Caco | MDA-MB 231 | HCT 116 | PC3 | NCI | | Kinases | | |
| | IC50 μM | (liver) | (colon) | (breast) | (colon) | (prostate) | (lung) | fibroblast | DYRK1A | CDK5 | GSK3 |
| 17 | 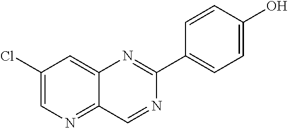 [SMILES: Oc1ccc(-c2ncc3ncc(Cl)cc3n2)cc1] | >25 | >25 | >25 | >25 | >25 | >25 | >25 | 16 | 5.1 | >100 |
| 20 | 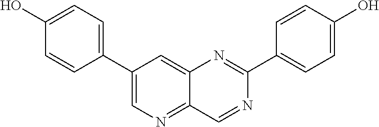 [SMILES: Oc1ccc(-c2cnc3cnc(-c4ccc(O)cc4)nc3c2)cc1] | 12 | 7 | >25 | 12 | 25 | >25 | >25 | 0.93 | 0.12 | 26 |
| 21 | 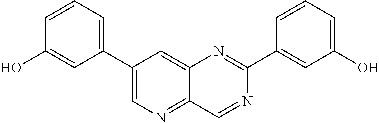 [SMILES: Oc1cccc(-c2cnc3cnc(-c4cccc(O)c4)nc3c2)c1] | >25 | 20 | >25 | >25 | 25 | >25 | >25 | 3.4 | 2.6 | >100 |
| 22 | 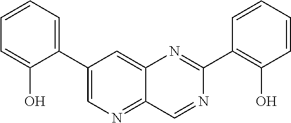 [SMILES: Oc1ccccc1-c1cnc2cnc(-c3ccccc3O)nc2c1] | >25 | >25 | >25 | >25 | >25 | 2 | >25 | >10 | >10 | >10 |
| 23 | 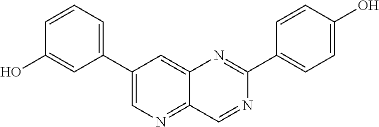 [SMILES: Oc1ccc(-c2ncc3ncc(-c4cccc(O)c4)cc3n2)cc1] | 10 | 4 | >25 | 7 | 9 | 20 | >25 | 2.4 | 0.11 | >100 |
| 24 | 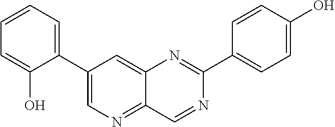 [SMILES: Oc1ccc(-c2ncc3ncc(-c4ccccc4O)cc3n2)cc1] | 25 | 20 | >25 | 20 | 20 | >25 | >25 | 2.7 | 0.09 | ≧100 |
| 25 | 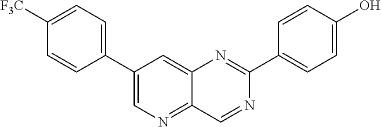 [SMILES: Oc1ccc(-c2ncc3ncc(-c4ccc(C(F)(F)F)cc4)cc3n2)cc1] | 25 | >25 | >25 | >25 | >25 | 20 | >25 | 24 | 0.97 | ≧100 |
| 26 | 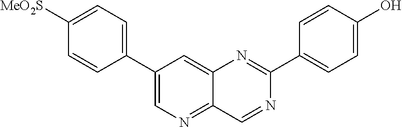 [SMILES: CS(=O)(=O)c1ccc(-c2cnc3cnc(-c4ccc(O)cc4)nc3c2)cc1] | >25 | >25 | >25 | >25 | >25 | >25 | >25 | >10 | 0.48 | >100 |
| 32 | 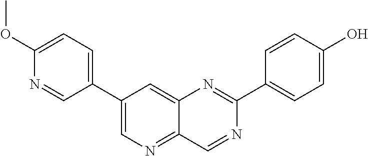 [SMILES: COc1ccc(-c2cnc3cnc(-c4ccc(O)cc4)nc3c2)cn1] | >25 | >25 | 25 | >25 | >25 | 25 | 10 | >10 | 1.2 | >10 |
| 33 | 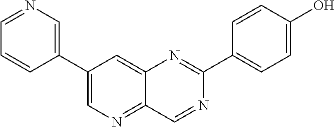 [SMILES: Oc1ccc(-c2ncc3ncc(-c4cccnc4)cc3n2)cc1] | >25 | >25 | >25 | >25 | >25 | >25 | >25 | >10 | 0.65 | >10 |
| 34 | 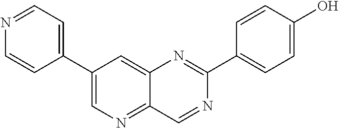 [SMILES: Oc1ccc(-c2ncc3ncc(-c4ccncc4)cc3n2)cc1] | >25 | >25 | >25 | 10 | >25 | 5 | 15 | >10 | 0.13 | >10 |
| 35 | 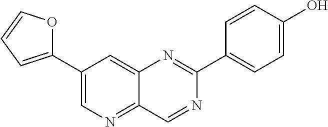 [SMILES: Oc1ccc(-c2ncc3ncc(-c4ccco4)cc3n2)cc1] | 15 | >25 | 4 | 5 | 20 | 7 | 6 | >10 | 1.8 | >10 |
| 36 | 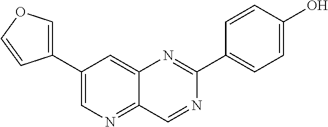 [SMILES: Oc1ccc(-c2ncc3ncc(-c4ccoc4)cc3n2)cc1] | >25 | >25 | >25 | 10 | >25 | >25 | 20 | >10 | 2.4 | >10 |
| 36 | 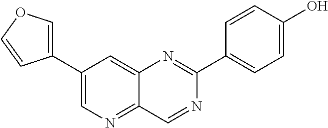 [SMILES: Oc1ccc(-c2ncc3ncc(-c4ccoc4)cc3n2)cc1] | 20 | 12 | 8 | 10 | 25 | 10 | 15 | >10 | 0.55 | >10 |
| 37 | 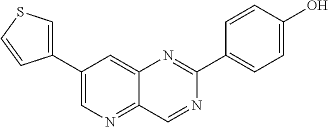 [SMILES: Oc1ccc(-c2ncc3ncc(-c4ccsc4)cc3n2)cc1] | >25 | >25 | >25 | 15 | >25 | 10 | >25 | >10 | 0.12 | >10 |
| 38 | 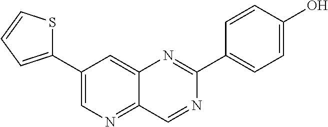 [SMILES: Oc1ccc(-c2ncc3ncc(-c4cccs4)cc3n2)cc1] | >25 | >25 | >25 | 4 | 25 | 3 | 2 | >10 | 0.11 | >10 |
| 47 | 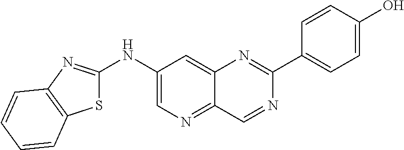 [SMILES: Oc1ccc(-c2ncc3ncc(Nc4nc5ccccc5s4)cc3n2)cc1] | >25 | >25 | >25 | >25 | >25 | >25 | >25 | >10 | 5.3 | >10 |
| 48 | 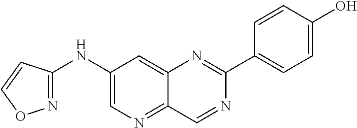 [SMILES: Oc1ccc(-c2ncc3ncc(Nc4ccon4)cc3n2)cc1] | 25 | 20 | 12 | 9 | 20 | 3 | 15 | 2 | 2.1 | >10 |
| 50 | 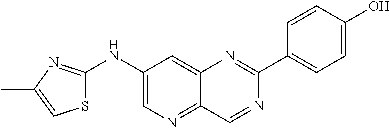 [SMILES: Cc1csc(Nc2cnc3cnc(-c4ccc(O)cc4)nc3c2)n1] | 10 | >25 | 25 | >25 | >25 | 25 | >25 | >10 | >10 | >10 |
| 51 | 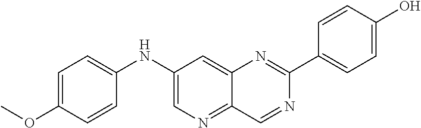 [SMILES: COc1ccc(Nc2cnc3cnc(-c4ccc(O)cc4)nc3c2)cc1] | 15 | 12 | 15 | 15 | 2 | 10 | 10 | >10 | 1.8 | 7.3 |
| 52 | 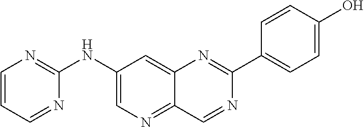 [SMILES: Oc1ccc(-c2ncc3ncc(Nc4ncccn4)cc3n2)cc1] | 25 | 5 | 2.5 | 2 | 1.5 | 1.5 | 2 | >10 | 7.6 | >10 |
| 53 | 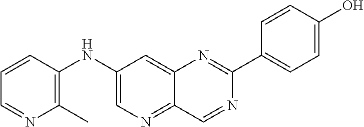 [SMILES: Cc1ncccc1Nc1cnc2cnc(-c3ccc(O)cc3)nc2c1] | 12 | 15 | 9 | >25 | 6 | 15 | 10 | >10 | >10 | >10 |
| 54 | 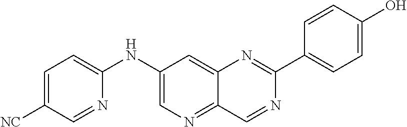 [SMILES: N#Cc1ccc(Nc2cnc3cnc(-c4ccc(O)cc4)nc3c2)nc1] | >25 | >25 | 12 | >25 | >25 | 20 | >25 | >10 | 4 | 5.5 |
| 56 | 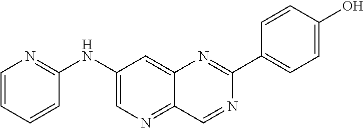 [SMILES: Oc1ccc(-c2ncc3ncc(Nc4ccccn4)cc3n2)cc1] | 12 | 10 | 10 | 1.5 | 3 | 0.15 | 2 | >10 | 5 | >10 |
| 59 | 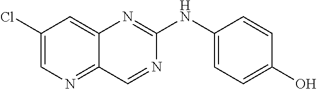 [SMILES: Oc1ccc(Nc2ncc3ncc(Cl)cc3n2)cc1] | 12 | >25 | 6 | 2 | 12 | 10 | 5 | >10 | 1.1 | >10 |
| 60 | 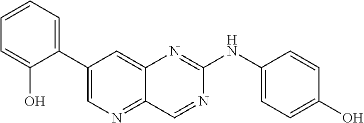 [SMILES: Oc1ccc(Nc2ncc3ncc(-c4ccccc4O)cc3n2)cc1] | 6 | 4 | 10 | 8 | 20 | 5 | 5 | >10 | 3.7 | >10 |
| 61 | 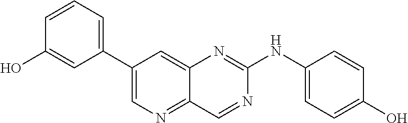 [SMILES: Oc1ccc(Nc2ncc3ncc(-c4cccc(O)c4)cc3n2)cc1] | 10 | 12 | 9 | 12 | 7 | 4 | 2 | >10 | 0.71 | 4.5 |
| 62 | 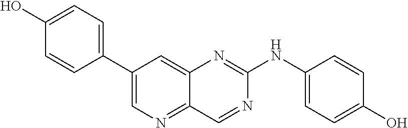 [SMILES: Oc1ccc(Nc2ncc3ncc(-c4ccc(O)cc4)cc3n2)cc1] | 12 | 15 | 10 | 12 | 15 | 4 | 5 | >10 | 0.84 | 7.1 |
| 83 | 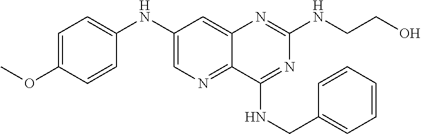 [SMILES: COc1ccc(Nc2cnc3c(NCc4ccccc4)nc(NCCO)nc3c2)cc1] | 5 | 8 | 10 | 5 | 12 | 6 | 15 | | | |
| 84 | 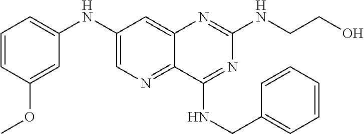 [SMILES: COc1cccc(Nc2cnc3c(NCc4ccccc4)nc(NCCO)nc3c2)c1] | 3 | 4 | 15 | 5 | 5 | 3 | 5 | | | |
| 85 | 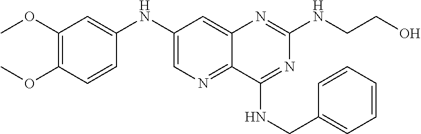 [SMILES: COc1ccc(Nc2cnc3c(NCc4ccccc4)nc(NCCO)nc3c2)cc1OC] | 1.5 | 4 | 15 | 4 | 4 | 2 | 5 | >10 | >10 | >10 |
| 86 | 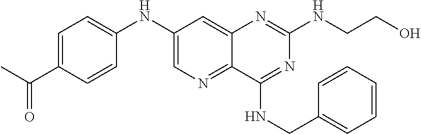 [SMILES: CC(=O)c1ccc(Nc2cnc3c(NCc4ccccc4)nc(NCCO)nc3c2)cc1] | 2 | 3 | 15 | 1 | 3 | 2 | 5 | >10 | >10 | >10 |
| 87 | 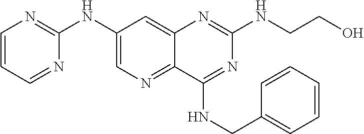 [SMILES: OCCNc1nc(NCc2ccccc2)c2ncc(Nc3ncccn3)cc2n1] | 7 | 8 | 15 | 6 | 15 | 6 | 15 | | | |
| 89 | 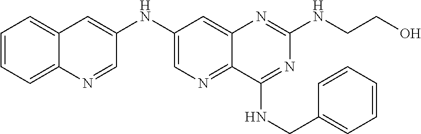 [SMILES: OCCNc1nc(NCc2ccccc2)c2ncc(Nc3cnc4ccccc4c3)cc2n1] | 3 | 2 | 3 | 0.5 | 3 | 2 | 2 | | | |
| 90 | 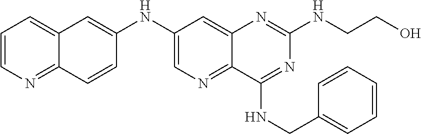 [SMILES: OCCNc1nc(NCc2ccccc2)c2ncc(Nc3ccc4ncccc4c3)cc2n1] | 2 | 4 | 4 | 1.2 | 5 | 2 | 1.5 | | | |

1. A compound of the following general formula (I): 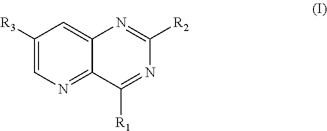
wherein:
R₁ is selected from the group consisting of:
hydrogen,
halogens,
(hetero)aryls comprising from 5 to 30 carbon atoms,
groups —NRₐR_{b}, Rₐ and R_{b} being independently selected from the group consisting of hydrogen, alkyls comprising from 1 to 10 carbon atoms, aryls comprising from 6 to 30 carbon atoms and arylalkyls comprising from 6 to 30 carbon atoms,
R₂ is selected from the group consisting of:
halogens,
(hetero)aryls comprising from 5 to 30 carbon atoms,
groups —NR′ₐR′_{b}, R′ₐ and R′_{b} being independently selected from the group consisting of hydrogen, alkyls comprising from 1 to 10 carbon atoms, aryls comprising from 6 to 30 carbon atoms and arylalkyls comprising from 6 to 30 carbon atoms,
R₃ is selected from the group consisting of:
halogens,
(hetero)aryls comprising from 5 to 30 carbon atoms,
groups —NR′ₐR′_{b}, R′ₐ and R′_{b} being independently selected from the group consisting of hydrogen, alkyls comprising from 1 to 10 carbon atoms, aryls or heteroaryls comprising from 6 to 30 carbon atoms and arylalkyls comprising from 6 to 30 carbon atoms,
or R″ₐ and R″_{b} forming with the nitrogen atom bearing R″ₐ and the group CO a heterocycle comprising from 6 to 10 atoms, and
groups —N(R″ₐ)CON(R″_{b}), R″ₐ and R″_{b} being as defined above,
as well as its pharmaceutically acceptable salts, its hydrates or its polymorphic crystalline structures, its racemates, diastereoisomers or enantiomers, except the compound 1-(2,4-diaminopyrido[3,2-d]pyrimidin-7-yl)-3,6,6-trimethyl-6,7-dihydro-1H-indol-4(5H)-one.

2. The compound of formula (I) according to claim 1, wherein R₁ is selected from the group consisting of:
hydrogen,
halogens,
(hetero)aryls comprising from 5 to 30 carbon atoms.

3. The compound of formula (I) according to claim 1, wherein R₁ represents a phenyl group.

4. The compound of formula (I) according to claim 1, wherein R₂ represents a phenyl group.

5. The compound of formula (I) according to claim 1, wherein R₂ represents a phenyl group substituted with a group OR_{α}, R_{α} representing H or an alkyl group comprising from 1 to 10 carbon atoms.

6.-12. (canceled)

13. The compound of formula (I) according to claim 1, wherein R₁ represents H.

14. The compound of formula (I) according to claim 1, wherein R₁ represents H, R₂ represents a phenyl group and R₃ represents a halogen or a phenyl group.

15. The compound of formula (I) according to claim 1, wherein R₁ represents H, R₂ represents a phenyl group substituted with a group OR_{α}, R_{α} representing H or an alkyl group, comprising from 1 to 10 carbon atoms, and R₃ represents a halogen or a phenyl group substituted with a group OR_{α}, R_{α} representing H or an alkyl group comprising from 1 to 10 carbon atoms.

16. The compound according to claim 1, of the following formula (I-3): 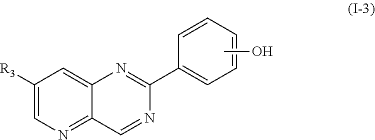
R₃ being as defined in claim 1.

17. The compound according to claim 1, of the above formula (I-3), wherein R₃ is selected from the group consisting of the following groups:
halogen,
furanyl,
thiophenyl,
pyridyl,
phenyl,
benzothiazolyl, and
a group —NHR″_{b}, R″_{b} being selected from the group consisting of the following groups:
phenyl,
pyridyl,
pyrimidinyl,
thiazolyl, and
isoxazolyl.

18. The compound of formula (I) according to claim 1, wherein R₁ represents NHBn.

19. The compound of formula (I) according to claim 1, wherein R₂ represents a —NH—(CH₂)₂—OH group.

20. The compound according to claim 1, of the following formula (I-4): 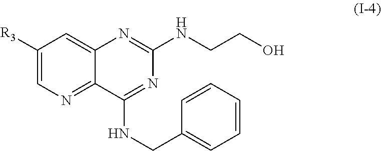
R₃ being as defined in claim 1.

21. A method for treating or preventing diseases related to a deregulation of CDK1, CDK5, GSK3 and/or DYRK1A kinases comprising a step of administering a pharmaceutically acceptable amount of a compound of formula (I) according to claim 1 to a patient in need thereof.

22. The method according to claim 21, wherein the disease is selected from the group consisting of cancers, Alzheimer's disease, Parkinson's disease, brain traumas, cerebravascular strokes, renal polycystoses, amyotrophic lateral scleroses, viral infections, auto-immune diseases, neurodegenerative disorders, psoriasis, asthma, atopical dermatitises, trisomia 21 and glomerulonephrites.
